(19) United States
(12) Patent Application Publication
(10) Pub. No.: US 2021/0395392 A1
(43) Pub. Date: Dec. 23, 2021

(54) ANTI-MERTK ANTIBODIES FOR TREATING CANCER

(71) Applicant: BRISTOL-MYERS SQUIBB COMPANY, Princeton, NJ (US)

(72) Inventors: Thomas E. Spires, Jr., Howell, NJ (US); Michael Quigley, San Carlos, CA (US); Virginie Lafont, Lawrence Twp, NJ (US); Ginger C. Rakestraw, Somerville, MA (US); Linda Liang, Mountain View, CA (US); Karen A. Augustine-Rauch, New Hope, PA (US)

(21) Appl. No.: 17/284,119

(22) PCT Filed: Oct. 8, 2019
(86) PCT No.: PCT/US2019/055155

§ 371 Date: Apr. 9, 2021

Related U.S. Application Data
(60) Provisional application No. 62/743,507, filed on Oct. 9, 2018.

(51) Int. Cl. C07K 16/40; A61K 47/68; A61P 35/00; C07K 16/28

(52) U.S. Cl. CPC C07K 16/40; A61K 47/6803; A61K 47/6871; A61P 35/00; A61K 2039/545; C07K 2317/92; C07K 2317/33; C07K 2317/31; C07K 16/2818

(57) ABSTRACT

This disclosure provides isolated antibodies that bind specifically to MerTK expressed on the surface of a cell and inhibit efferocytosis by the MerTK-expressing cell. The disclosure provides methods for treating a subject afflicted with a cancer comprising administering to the subject a therapeutically effective amount of an anti-MerTK antibody as monotherapy or in combination with a checkpoint inhibitor, such as an anti-PD-1 or anti-PD-L1 antibody.

### CROSS REFERENCE TO RELATED APPLICATIONS

This application claims the benefit U.S. Provisional Application No. 62/743,507, filed Oct. 9, 2018, the entire contents of which are hereby incorporated herein by reference.

Throughout this application, various publications are referenced in parentheses by author name and date, or by Patent No. or Patent Publication No. Full citations for these publications may be found at the end of the specification immediately preceding the claims. The disclosures of these publications are hereby incorporated in their entireties by reference into this application in order to more fully describe the state of the art as known to those skilled therein as of the date of the invention described and claimed herein. However, these disclosures are incorporated into the present application only to the extent that no conflict exists between the information incorporated by reference and the information provided by explicit disclosure in the present application. Moreover, the citation of a reference herein should not be construed as an acknowledgement that such reference is prior art to the present invention.

### SEQUENCE LISTING

The present application contains a Sequence Listing which has been submitted electronically in ASCII format and is hereby incorporated herein by reference in its entirety. The ASCII copy was created on Oct. 4, 2019, is named 12970WOPCT_Seq_List_ST25.txt, and is 135,398 bytes in size.

### FIELD OF THE INVENTION

This disclosure relates to monoclonal antibodies (mAbs) that bind specifically to proto-oncogene tyrosine-protein kinase MER (MerTK), and methods for treating a cancer in a subject comprising administering to the subject an anti-MerTK antibody (Ab) as monotherapy or in combination with an anticancer agent such as an immune checkpoint inhibitor, a chemotherapeutic agent and/or radiation therapy.

### BACKGROUND OF THE INVENTION

Human cancers harbor numerous genetic and epigenetic alterations, generating neoantigens potentially recognizable by the immune system (Chakravarthi et al., 2016). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system makes immunotherapy unique among all cancer treatment modalities.

The past decade has witnessed the development of specific immune checkpoint pathway inhibitors for treating cancer (Chen and Mellman, 2013; Lesokhin et al., 2015), including the development of an Ab, ipilimumab (YERVOY®), that binds to and inhibits Cytotoxic T-Lymphocyte Antigen-4 (CTLA-4) for the treatment of patients with advanced melanoma, and Abs such as nivolumab (OPDIVO®) and pembrolizumab (KEYTRUDA®) that bind specifically to the PD-1 receptor and block the inhibitory PD-1/PD-1 ligand (PD-L1) signaling pathway (Iwai et al., 2017). This pathway can also be disrupted by Abs, including atezolizumab (TECENTRIQ®), durvalumab (IMFINZI®), and avelumab (BAVENCIO®), that bind specifically to PD-L1.

Nivolumab is a fully human immunoglobulin (Ig) G4 (S228P) monoclonal antibody (mAb) that selectively prevents interaction with the PD-1 ligands, PD-L1 and PD-L2 (U.S. Pat. No. 8,008,449; Wang et al., 2014), thereby blocking the down-regulation of antigen-specific T cell responses directed against both foreign (including tumor) and self antigens and enhancing an immune response against these antigens. Nivolumab has received approval recently for several cancers including melanoma, lung cancer, renal cell carcinoma, classical Hodgkin lymphoma, head and neck cancer, urothelial carcinoma, MSI-H or dMMR metastatic colorectal cancer, and hepatocellular carcinoma, and is currently being clinically evaluated as monotherapy or in combination with other anti-cancer agents in additional tumor types. However, only a small percentage of patients, typically less than around 25%, benefit from treatment with checkpoint inhibitors, and considerable efforts are now focused on improving the efficacy of immunotherapy using combinations of checkpoint inhibitors and other anti-cancer agents or therapies. Because PD-1/PD-L1 inhibitors have proven to be so successful in treating a broad spectrum of cancers, they are perceived to be the likely backbone of various future drug combinations in immuno-oncology and a race is on to develop the most effective combinations (see, e.g., Mahoney et al., 2015; Ott et al., 2017).

MerTK (c-Mer Tyrosine Kinase; proto-oncogene tyrosine-protein kinase MER) is a member of the TAM (Tyro3/Ax1/Mer) family of protein receptor tyrosine kinases (RTKs), which exhibit a similar overall structure comprising from the N-termini two Ig-like C2-type domains, two fibronectin (Fn) type-III domains, followed by a hydrophobic transmembrane domain and an intracellular tyrosine kinase domain. The two Ig-like domains serve as the ligand-binding regions of the TAMs.

TAM RTKs are ectopically expressed or overexpressed in a wide variety of human cancers, especially hematological and epithelial malignancies, and there is growing interest in understanding the role of TAM receptors in modulating the anti-tumor immune response. In the tumor microenvironment, MerTK is mainly expressed on tumor-associated macrophages, with lower expression on monocytes and dendritic cells (DCs). Rather than functioning as oncogenic drivers, the induction of TAM RTKs in tumor cells predominantly promotes survival, chemoresistance and motility (Linger et al., 2008; Graham et al., 2014). Although MerTK knockdown only modestly promotes apoptosis and slows proliferation in cell cultures, the effect is more pronounced under stressful conditions such as when combined with serum starvation or growth in soft agar or xenografts (Lee-Sherick et al., 2013; Linger et al., 2013). This suggests that TAM survival signals may be particularly important in the tumor microenvironment, in which limited oxygen and nutrient supplies exacerbate the proteotoxic and genotoxic conditions.

Growth Arrest-Specific Protein 6 (Gas6) and Protein 51 (PROS1) are the best studied ligands for this receptor family and serve as bridging molecules that bind to phosphatidyl serine on the outer membrane of apoptotic cells through their N-terminal GLA domains and directly to MerTK through their C-terminal domains (Graham et al., 2014). These ligands bind to, and activate, the TAM receptors (Stitt et al., 1995). Two other reported ligands, Tubby and Tubby-Like Protein 1 (Tulp1) also act similarly as bridging ligands for MerTK through N-terminal MPD (minimal phagocytotic determinant) domains and highly conserved C-terminal PPBD (phagocytosis prey binding domain) domains which engage apoptotic cells (Caberoy et al., 2010). There have also been reports that galectin-3 (Gal-3) can also bind directly to MerTK but this putative interaction is less well understood of (Caberoy et al., 2012).

Other than some hematological and epithelial cancers, MerTK is expressed predominantly on tumor-associated macrophages, tolerogenic dendritic cells and natural killer (NK) cells (Graham et al., 2014). It is also expressed on tissue-resident macrophage populations that are professional phagocytotic cells of the immune system, and normal epithelial cells such as red pulp macrophages and the retinal epithelium. The ligands are expressed by many cells including myeloid cells, activated T cells and by many cancer cells/types (Graham et al., 2014). Often the cells expressing MerTK or other TAM family receptors are the same cells expressing one or more ligands, resulting in potential autocrine-mediated activation. The expression and binding of the various ligands to the TAM family of receptors regulates numerous physiological processes including cell survival, migration, differentiation, and efferocytosis, the process of specifically targeting and phagocytosing apoptotic cells.

MerTK expression on macrophages is crucial for their phagocytotic function in both healthy and injured tissues. MerTK is a key mediator of efferocytosis, and is thought to contribute to immunosuppression and tolerance in the tumor microenvironment. It has been shown that overexpression of MerTK is sufficient to instill gain of function capacity to cell lines and enable them to efficiently engulf apoptotic cells and that loss of function is attained by knocking out MerTK expression (Nguyen et al., 2014).

Published reports using MerTK^{-/-} mice have demonstrated immune-mediated, enhanced anti-tumor activity in immunogenic settings such as the PyVMT breast cancer model and increased tumor growth delay even in difficult-to-treat settings, such as the B16F10 melanoma model (Cook et al., 2013). Consistent with the proposed mechanisms in play with MerTK blockade, it has also been shown that CD8 Teff cell function is required for these anti-tumor benefits (Cook et al., 2013). An important feature of macrophages ingesting apoptotic cells is their subsequent propensity to downregulate the generation of proinflammatory cytokines and upregulate factors associated with immunosuppression. Various studies support the idea that MerTK-dependent phagocytosis of apoptotic tumor cells leads to a signaling cascade that favors tumor-promoting polarization of macrophages, and these pro-tumorigenic programs augment production of immunosuppressive cytokines that aid tumor growth (see Akalu et al., 2017). In addition, it has been shown that blockade of efferocytosis with phosphatidyl serine blocking agents (e.g., annexin V) both in vitro and in vivo leads to a reduction in immunosuppressive factors (e.g., TGF-β), increased proinflammatory factors (e.g., TNF-α) and enhanced macrophage-mediated T cell proliferation (Barker et al. 2002; Bondanza et al., 2004). These data, among others, suggest the possibility that blockade of efferocytosis using antagonistic ligand-blocking Abs directed specifically against MerTK may be effective as anti-cancer therapeutics. Thus, the present study was undertaken to identify Abs that bind to MerTK with high affinity and inhibit efferocytosis for use in treating cancer. Such Abs may be particularly effective in combination with agents that reinvigorate T cell responses, such as checkpoint inhibitors, and/or treatments that induce apoptotic responses in the tumor microenvironment, such as certain chemotherapeutic compounds and radiation therapies (Jinushi et al., 2013).

A recent PCT publication (WO 2016/106221) describes the isolation of mAbs that specifically bind to human MerTK (or both human and mouse MerTK) with high affinity, inhibit Gas6 binding to MerTK, and agonize MerTK signaling on endothelial cells. WO 2016/106221 also provides methods for treating cancer by administering to a subject an Ab that specifically binds to MerTK and agonizes MerTK signaling on endothelial cells, i.e., activates MerTK phosphorylation on endothelial cells. Two mAbs were shown to inhibit tumor progression in a mouse model of human breast cancer. The ability of a MerTK agonist to treat cancer was rationalized on the basis that Gas-6 activation of MerTK on endothelial cells results in inhibition of endothelial cell recruitment by cancer cells, which is a key feature of cancer cells that allows for tumor angiogenesis, tumor growth, and metastasis. Thus, a compound that activates MerTK signaling on endothelial cells but not cancer cells may be effective in reducing tumor angiogenesis and metastasis. A second PCT publication (WO 2019/005756) describes the production of Ab-drug conjugates of M6 and M19 and their use in treating cancer.

The present disclosure relates to the production of anti-MerTK Abs and evaluation of their efficacy in, and suitability for, treating cancer. The disclosure also relates to an evaluation of the efficacy of treating cancer by anti-MerTK Abs in combination with checkpoint blockade, for example, inhibition of the PD-1/PD-L1 signaling pathways. The combination of the mechanisms of action of anti-MerTK and anti-PD-1/anti-PD-L1 offers a unique opportunity to increase tumor cell killing.

### SUMMARY OF THE INVENTION

The present invention provides isolated Abs, preferably mAbs, that bind to MerTK expressed on the surface of a cell and exhibit various functional properties, including properties that are desirable in a therapeutic Ab. These properties include high affinity binding to MerTK, inhibiting efferocytosis by the MerTK-expressing cell, principally a macrophage, inhibiting binding of growth arrest-specific protein 6 (Gas6) to hMerTK, disrupting the interaction between MerTK and Gas6, inhibiting MerTK/Gas6 signaling, inhibiting growth of tumor cells in a subject when administered to the subject as monotherapy or in combination with another anti-cancer agent, and treating a subject afflicted with a cancer when administered to the subject as monotherapy or in combination with another anti-cancer agent. In certain embodiments, a disclosed anti-MerTK mAb binds to a MerTK which is a human MerTK (hMerTK), cynomolgus monkey MerTK (cMerTK), murine MerTK (mMerTK), or a combination of these MerTK targets. In preferred embodiments, the subject is a human subject.

In certain embodiments, an anti-MerTK Ab inhibits efferocytosis by a hMerTK-expressing cell with an IC₅₀ of about 1 nM or lower, preferably between about 0.04 nM and about 0.7 nM. In certain other embodiments, the anti-MerTK Ab inhibits hMerTK/Gas6 signaling with an IC₅₀ of about 10 nM or lower, preferably between about 0.1 nM and about 5 nM. In further embodiments, the anti-MerTK Ab binds specifically to hMerTK with a K_{D} of about 70 nM or lower, preferably between about 2 nM and about 25 nM. In yet other embodiments, the anti-MerTK Ab binds specifically to hMerTK, cMerTK and mMerTK with high affinity.

The anti-MerTK Abs provided herein have been assigned to three epitope bins. In certain embodiments, the Ab belongs to Bin 1. Bin 1 Abs bind to the first Ig domain of MerTK within a region spanning approximately amino acids 105 to 165. In preferred embodiments, the Ab belongs to Bin 2. Bin 2 Abs bind to the second Ig domain within a region spanning approximately amino acids 195 to 270. In further embodiments, the Ab belongs to Bin 3. Bin 3 Abs binds to the fibronectin (Fn) domains within a region spanning approximately amino acids 420 to 490.

This disclosure provides an isolated Ab, preferably a mAb, or an antigen-binding portion thereof, which specifically binds to hMerTK expressed on the surface of a cell and comprises the CDR1, CDR2 and CDR3 domains in each of the following pairs of heavy and light chain variable regions:

(a) a V_{H} comprising consecutively linked amino acids having the sequence set forth as SEQ ID NO: 217 and a V_{L} comprising consecutively linked amino acids having the sequence set forth as SEQ ID NO: 218;

(b) a V_{H} comprising consecutively linked amino acids having the sequence set forth as SEQ ID NO: 221 and a V_{L} comprising consecutively linked amino acids having the sequence set forth as SEQ ID NO: 222;

(c) a V_{H} comprising consecutively linked amino acids having the sequence set forth as SEQ ID NO: 225 and a V_{L} comprising consecutively linked amino acids having the sequence set forth as SEQ ID NO: 226;

(d) a V_{H} comprising consecutively linked amino acids having the sequence set forth as SEQ ID NO: 229 and a V_{L} comprising consecutively linked amino acids having the sequence set forth as SEQ ID NO: 230;

(e) a V_{H} comprising consecutively linked amino acids having the sequence set forth as SEQ ID NO: 233 and a V_{L} comprising consecutively linked amino acids having the sequence set forth as SEQ ID NO: 234;

(f) a V_{H} comprising consecutively linked amino acids having the sequence set forth as SEQ ID NO: 237 and a V_{L} comprising consecutively linked amino acids having the sequence set forth as SEQ ID NO: 238;

(g) a V_{H} comprising consecutively linked amino acids having the sequence set forth as SEQ ID NO: 241 and a V_{L} comprising consecutively linked amino acids having the sequence set forth as SEQ ID NO: 242;

(h) a V_{H} comprising consecutively linked amino acids having the sequence set forth as SEQ ID NO: 245 and a V_{L} comprising consecutively linked amino acids having the sequence set forth as SEQ ID NO: 246;

(i) a V_{H} comprising consecutively linked amino acids having the sequence set forth as SEQ ID NO: 249 and a V_{L} comprising consecutively linked amino acids having the sequence set forth as SEQ ID NO: 250;

(j) a V_{H} comprising consecutively linked amino acids having the sequence set forth as SEQ ID NO: 253 and a V_{L} comprising consecutively linked amino acids having the sequence set forth as SEQ ID NO: 254;

(k) a V_{H} comprising consecutively linked amino acids having the sequence set forth as SEQ ID NO: 255 and a V_{L} comprising consecutively linked amino acids having the sequence set forth as SEQ ID NO: 256; or

(l) a V_{H} comprising consecutively linked amino acids having the sequence set forth as SEQ ID NO: 257 and a V_{L} comprising consecutively linked amino acids having the sequence set forth as SEQ ID NO: 258.

The sequences of the variable regions may be defined by a variety of methods, including the Kabat, Chothia, AbM, contact and IMGT definitions.

The disclosure also provides an isolated nucleic acid encoding any of the Abs or antigen-binding portions thereof described herein. The disclosure provides an expression vector comprising said isolated nucleic acid, and a host cell comprising said expression vector. This host cell may be used in a method for preparing an anti-MerTK mAb or an antigen-binding portion thereof, which method comprises expressing the mAb or antigen-binding portion thereof in said host cell and isolating the mAb or antigen-binding portion thereof from the host cell.

In certain embodiments, the present disclosure provides a method for treating a subject afflicted with a cancer comprising administering to the subject a therapeutically effective amount of any of the anti-MerTK mAbs or antigen-binding portions described herein, such that the subject is treated. In other embodiments, the disclosure provides a method for inhibiting growth of tumor cells in a subject, comprising administering to the subject a therapeutically effective amount of any of the anti-MerTK mAbs or antigen-binding portions described herein, such that growth of tumor cells in the subject is inhibited. In certain embodiments of these methods, the anti-MerTK mAb inhibits efferocytosis by the MerTK-expressing cell. In certain other embodiments, the anti-MerTK mAb inhibits binding of MerTK to its ligand and inhibits MerTK/ligand signaling.

This disclosure further provides a method for treating a subject afflicted with a cancer comprising administering to the subject a combination of therapeutically effective amounts of (a) any of the anti-MerTK mAbs or antigen-binding portions described herein, and (b) an additional therapeutic agent for treating cancer. In certain preferred embodiments, the additional therapeutic agent is an antagonistic Ab or antigen-binding portion thereof that binds specifically to PD-1 or to PD-L1.

Other features and advantages of the instant invention will be apparent from the following detailed description and examples which should not be construed as limiting. The contents of all cited references, including scientific articles, GenBank entries, patents and patent applications cited throughout this application are expressly incorporated herein by reference.

### BRIEF DESCRIPTION OF THE FIGURES

FIGS. 1A-1C show the effects on tumor growth of the combination of a mouse anti-mPD-1 Ab (4H2) and a mouse anti-mMerTK Ab compared to anti-PD-1 Ab therapy alone, as measured by changes in the tumor volumes in 10 individual mice treated with the Abs in a MC38 mouse colon adenocarcinoma tumor model: FIG. 1A, control mouse IgG1 Ab; FIG. 1B, anti-mouse PD-1 Ab (clone 4H2); FIG. 1C, combination of an anti-PD-1 Ab and an anti-mouse MerTK (clone 4E9) Ab. Seven out of 10 mice were cured, i.e., showed 100% shrinkage of the tumor, in the combination group whereas none of the mice treated with the anti-PD-1 Ab alone was cured.

FIG. 2 shows the resistance of MC38 mice, cured by treatment with anti-MerTK in combination with anti-PD1, to rechallenge with tumors. All seven of the rechallenged mice were resistant to MC38 tumor growth.

FIGS. 3A-3H show the effects on tumor growth of the combination of an anti-mouse PD-1 Ab (4H2) and different mouse anti-mMerTK Abs (4E9 and 2D9) having different FcR effector functions compared to anti-PD-1 Ab therapy alone, as measured by changes in the tumor volumes in individual mice treated with the Abs in the MC38 tumor model: FIG. 1A, control mouse IgG1 Ab; FIG. 1B, anti-mMerTK Ab (2D9-IgG1); FIG. 1C, anti-mMerTK Ab (2D9-D265A); FIG. 1D, anti-mMerTK Ab (4E9-D265A); FIG. 1E, anti-mPD-1 Ab; FIG. 1F, combination of an anti-mPD-1 Ab and an anti-mMerTK Ab (2D9-IgG1); FIG. 1G, combination of an anti-mPD-1 Ab and an anti-mMerTK Ab (2D9-D265A); FIG. 1H, combination of an anti-mPD-1 Ab and an anti-mMerTK Ab (4E9-D265A). Similar efficacy was observed with the two different anti-MerTK Abs irrespective of whether the FcR effector function was IgG1 or IgG1-D265A.

FIGS. 4A-4D show the effects on tumor growth of an anti-mPD-1 Ab (4H2) and an anti-mMerTK Ab used alone or in combination in a CT26 mouse colon carcinoma tumor model: FIG. 1A, control mouse IgG1 Ab; FIG. 1B, anti-mPD-1 Ab; FIG. 1C, anti-mMerTK Ab (4E9-IgG1); FIG. 1D, combination of the anti-mPD-1 Ab and an anti-mMerTK Ab (4E9-IgG1). Four out of 10 mice were cured in mice subjected to the combination treatment whereas none and one of the mice treated with anti-MerTK and anti-PD-1 Ab monotherapy, respectively, was cured.

FIGS. 5A-5D show the effects on tumor growth of an anti-mPD-1 Ab (4H2) and an anti-mMerTK Ab used alone or in combination in the MC38 tumor model: FIG. 1A, control mouse IgG1 Ab; FIG. 1B, anti-mPD-1 Ab; FIG. 1C, anti-mMerTK Ab (16B9-D265A); FIG. 1D, combination of the anti-mPD-1 Ab and an anti-mMerTK Ab (16B9-D265A). Seven out of 10 mice were cured in mice subjected to the combination treatment whereas none and one of the mice treated with anti-MerTK and anti-PD-1 Ab monotherapy, respectively, was cured.

### DETAILED DESCRIPTION OF THE INVENTION

The present invention relates to mAbs that bind specifically to MerTK and to methods for treating cancers in a patient comprising administering to the patient an anti-MerTK Ab alone or in combination with an anticancer agent such as an immune checkpoint inhibitor.

### Terms

In order that the present disclosure may be more readily understood, certain terms are first defined. As used in this application, except as otherwise expressly provided herein, each of the following terms shall have the meaning set forth below. Additional definitions are set forth throughout the application.

“Administering” refers to the physical introduction of a therapeutic agent or a composition comprising a therapeutic agent to a subject, using any of the various methods and delivery systems known to those skilled in the art. A preferred route for administration of therapeutic Abs such as anti-PD-1 and anti-MerTK Abs is intravenous administration. Other routes of administration include intramuscular, subcutaneous, intraperitoneal, or other parenteral routes of administration, for example by injection or infusion. The phrase “parenteral administration” as used herein means modes of administration other than enteral and topical administration. Administering can also be performed, for example, once, a plurality of times, and/or over one or more extended periods.

An “antibody” (Ab) shall include, without limitation, a glycoprotein immunoglobulin (Ig) which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region of an IgG Ab comprises three constant domains, Cm, CH2 and CH3. Each light chain comprises a light chain variable region (abbreviated herein as V_{L}) and a light chain constant region. The light chain constant region of an IgG Ab comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1-CDR1-FR2-CDR2-FR3-CDR3-FR4. The variable regions of the heavy and light chains contain a binding domain that interacts with an antigen. A variety of methods have been used to delineate the CDR domains within an Ab, including the Kabat, Chothia, AbM, contact, and IMGT definitions. The constant regions of the Abs may mediate the binding of the Ig to host tissues or factors, including various cells of the immune system (e.g., effector cells) and the first component (Clq) of the classical complement system.

An Ig may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the Ab class or subclass (e.g., IgM, IgG1, or IgG4) that is encoded by the heavy chain constant region genes. The term “antibody” includes, by way of example, both naturally occurring and non-naturally occurring Abs, monoclonal and polyclonal Abs, chimeric and humanized Abs, human or nonhuman Abs, wholly synthetic Abs, and single chain Abs. A nonhuman Ab may be humanized partially or fully by recombinant methods to reduce its immunogenicity in man. Where not expressly stated, and unless the context indicates otherwise, the term “antibody” also includes an antigen-binding fragment or an antigen-binding portion of any of the aforementioned Ig's, and includes a monovalent and a divalent fragment or portion, and a single chain Ab.

An “isolated” Ab refers to an Ab that is substantially free of other Abs having different antigenic specificities (e.g., an isolated Ab that binds specifically to MerTK is substantially free of Abs that bind specifically to antigens other than MerTK, such as Abs that bind to Axl or Tyro3). An isolated Ab that binds specifically to hMerTK may, however, have cross-reactivity to other antigens, such as MerTK polypeptides from different species such as mouse and cynomolgus monkey. Moreover, an isolated Ab may also mean an Ab that is purified so as to be substantially free of other cellular material and/or chemicals.

The term “monoclonal” Ab (mAb) refers to a non-naturally occurring preparation of Ab molecules of single molecular composition, i.e., Ab molecules whose primary sequences are essentially identical and which exhibit a single binding specificity and affinity for a particular epitope. A mAb is an example of an isolated Ab. MAbs may be produced by hybridoma, recombinant, transgenic or other techniques known to those skilled in the art.

A “chimeric” Ab refers to an Ab in which the variable regions are derived from one species and the constant regions are derived from another species, such as an Ab in which the variable regions are derived from a mouse Ab and the constant regions are derived from a human Ab.

A “human” mAb (HuMAb) refers to a mAb having variable regions in which both the framework and CDR regions are derived from human germline immunoglobulin sequences. Furthermore, if the Ab contains a constant region, the constant region also is derived from human germline immunoglobulin sequences. The human Abs of the invention may include amino acid residues not encoded by human germline immunoglobulin sequences (e.g., mutations introduced by random or site-specific mutagenesis in vitro or by somatic mutation in vivo). However, the term “human” Ab, as used herein, is not intended to include Abs in which CDR sequences derived from the germline of another mammalian species, such as a mouse, have been grafted onto human framework sequences. The terms “human” Abs and “fully human” Abs are used synonymously.

A “humanized” mAb refers to a mAb in which some, most or all of the amino acids outside the CDR domains of a non-human mAb are replaced with corresponding amino acids derived from human immunoglobulins. In one embodiment of a humanized form of an Ab, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the Ab to bind to a particular antigen. A “humanized” Ab retains an antigenic specificity similar to that of the original Ab.

An “anti-antigen” Ab refers to an Ab that binds specifically to an antigen. For example, an anti-PD-1 Ab is an Ab that binds specifically to PD-1, whereas an anti-MerTK Ab is an Ab that binds specifically to MerTK. As used herein, an “anti-PD-1/anti-PD-L1” Ab is an Ab that is used to disrupt the PD-1/PD-L1 signaling pathway, which may be an anti-PD-1 Ab or an anti-PD-L1 Ab.

An “antigen-binding portion” of an Ab (also called an “antigen-binding fragment”) refers to one or more fragments of an Ab that retain the ability to bind specifically to the antigen bound by the whole Ab.

“Binning” of Abs refers to a method of determining the epitope-binding characteristics of a library of antigen-specific Abs. Binning methods are commonly based on measuring competitive binding of each Ab in a library of Abs to their common antigen using techniques such as surface plasmon resonance (SPR), enzyme-linked immunoassay (ELISA) or flow cytometry. A competitive blocking profile is created for each Ab relative to the others in the library. An Ab's bin is defined using a reference Ab. If a second Ab is unable to bind to an antigen at the same time as the reference Ab, the second Ab is said to belong to the same bin as the reference Ab. Conversely, if a second Ab is capable of binding to an antigen at the same time as the reference Ab, the second Ab is said to belong to a separate bin. Abs belonging to the same bin generally bind to the same epitope region of an antigen, i.e., they may bind to identical or overlapping epitopes. However, in some cases Abs in the same bin may bind to separate epitopes but one Ab bound to its epitope sterically hinders the binding of the other Ab to its distinct epitope. Abs belonging to different bins generally bind to separate epitopes.

A “cancer” refers a broad group of various diseases characterized by the uncontrolled growth of abnormal cells in the body. Unregulated cell division and growth divide and grow results in the formation of malignant tumors that invade neighboring tissues and may also metastasize to distant parts of the body through the lymphatic system or bloodstream.

“Tyrosine-protein kinase Mer” (MerTK; also known in the art as, for example, Proto-oncogene c-Mer, Receptor tyrosine kinase MerTK, or Mer transmembrane receptor tyrosine kinase glycoform) is a transmembrane protein in the Tyro3/Axl/Mer (TAM) receptor tyrosine kinase (RTK) family. It is expressed on macrophages, natural killer (NK) cells, natural killer T (NKT) cells, and dendritic cells (DC), and is also often overexpressed or activated in a wide variety of cancers, including leukemia, non-small cell lung cancer, glioblastoma, melanoma, prostate cancer, breast cancer, colon cancer, gastric cancer, pituitary adenomas, and rhabdomyosarcomas. MerTK binds to several different ligands, growth arrest-specific 6 (Gas6) protein, protein S, tubby, tubby-like protein 1 (Tulp1), and galectin-3, all of which induce MerTK autophosphorylation. The term “MerTK” as used herein includes human MerTK (hMerTK), variants, isoforms, species homologs of hMerTK such as cynomolgus monkey MerTK (cMerTK) and mouse MerTK (mMerTK), and analogs having at least one common epitope with hMerTK. The complete hMerTK, cMerTK and mMerTK amino acid sequences can be found under GENBANK® Accession Nos. NP 006334.2, XP 005575320.1 and NP 032613.1, respectively.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, including the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

“Programmed Death-1” (PD-1) refers to an immunoinhibitory receptor belonging to the CD28 family that is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 amino acid sequence can be found under GENBANK® Accession No. U64863.

“Programmed Death Ligand-1” (PD-L1) is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GENBANK® Accession No. Q9NZQ7.

A “subject” includes any human or nonhuman animal. The term “nonhuman animal” includes, but is not limited to, vertebrates such as nonhuman primates, sheep, dogs, and rodents such as mice, rats and guinea pigs. In preferred embodiments, the subject is a human. The terms “subject” and “patient” are used interchangeably herein.

A “therapeutically effective amount” or “therapeutically effective dosage” of a drug or therapeutic agent is any amount of the drug or agent that, when used alone or in combination with another therapeutic agent, protects a subject against the onset of a disease or promotes disease regression evidenced by a decrease in severity of disease symptoms, an increase in frequency and duration of disease symptom-free periods, or a prevention or reduction of impairment or disability due to the disease affliction. In addition, the terms “effective” and “effectiveness” with regard to a treatment includes both pharmacological effectiveness and physiological safety. Pharmacological effectiveness refers to the ability of the drug to promote disease regression, e.g., cancer regression, in the patient. Physiological safety refers to an acceptable level of toxicity, or other adverse physiological effects at the cellular, organ and/or organism level (adverse effects) resulting from administration of the drug. The efficacy of a therapeutic agent can be evaluated using a variety of methods known to the skilled practitioner, such as in human subjects during clinical trials, in animal model systems predictive of efficacy in humans, or by assaying the activity of the agent in in vitro assays.

By way of example for the treatment of tumors, a therapeutically effective amount of an anti-cancer agent preferably inhibits cell growth or tumor growth by at least about 20%, preferably by at least about 40%, more preferably by at least about 60%, even more preferably by at least about 80%, and still more preferably by about 100% relative to untreated subjects. In preferred embodiments of the invention, tumor regression may be observed and continue for a period of at least about 30 days, more preferably at least about 60 days, or even more preferably at least about 6 months. Notwithstanding these ultimate measurements of therapeutic effectiveness, evaluation of immunotherapeutic drugs must also make allowance for “immune-related” response patterns.

An “immune-related” response pattern refers to a clinical response pattern often observed in cancer patients treated with immunotherapeutic agents that produce anti-tumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents may require long-term monitoring of the effects of these agents on the target disease.

A therapeutically effective amount of a drug includes a “prophylactically effective amount,” which is any amount of the drug that, when administered alone or in combination with an another therapeutic agent to a subject at risk of developing a disease (e.g., a subject having a pre-malignant condition who is at risk of developing a cancer) or of suffering a recurrence of the disease, inhibits the development or recurrence of the disease (e.g., a cancer). In preferred embodiments, the prophylactically effective amount prevents the development or recurrence of the disease entirely. “Inhibiting” the development or recurrence of a disease means either lessening the likelihood of the disease's development or recurrence, or preventing the development or recurrence of the disease entirely.

The use of the alternative (e.g., “or”) should be understood to mean either one, both, or any combination thereof of the alternatives. As used herein, the indefinite articles “a” or “an” should be understood to refer to “one or more” of any recited or enumerated component.

The term “about” refers to a numeric value, composition or characteristic that is within an acceptable error range for the particular value, composition or characteristic as determined by one of ordinary skill in the art, which will depend in part on how the value, composition or characteristic is measured or determined, i.e., the limitations of the measurement system. For example, “about” can mean a range of plus or minus 20%, more usually a range of plus or minus 10%. When particular values, compositions or characteristics are provided in the application and claims, unless otherwise stated, the meaning of “about” should be assumed to be within an acceptable error range for that particular value, composition or characteristic.

The term “substantially the same” or “essentially the same” refers to a sufficiently high degree of similarity between two or more numeric values, compositions or characteristics that one of skill in the art would consider the difference between these values, compositions or characteristics to be of little or no biological and/or statistical significance within the context of the property being measured. The difference between numeric values being measured may, for example, be less than about 50%, preferably less than about 30%, and more preferably less than about 10%.

As described herein, any concentration range, percentage range, ratio range or integer range is to be understood to include the value of any integer within the recited range and, when appropriate, fractions thereof (such as one tenth and one hundredth of an integer), unless otherwise indicated.

Various aspects of the invention are described in further detail in the following subsections.

Anti MerTK mAbs

In certain aspects, the present disclosure relates to isolated Abs, particularly mAbs or antigen-binding portions thereof, that specifically bind to MerTK expressed on the surface of a cell. The MerTK to which the mAbs bind includes hMerTK, the sequence of which is set forth as SEQ ID NO: 259; cMerTK, the sequence of which is set forth as SEQ ID NO: 260; and/or mMerTK, the sequence of which is set forth as SEQ ID NO: 261.

Inhibition of Efferocytosis by Anti MerTK mAbs

Efferocytosis by macrophages contributes to immunosuppression and tolerance in the tumor microenvironment (Nguyen et al., 2014; Akalu et al., 2017), and the inhibition of pathways involved in the clearance of apoptotic cells might enhance anti-tumorigenic responses. Indeed, blockade of efferocytosis has been shown to result in a reduction in immunosuppressive factors both in vitro and in vivo, and in enhanced macrophage-mediated T cell proliferation (Barker et al. 2002; Bondanza et al., 2004). In view of the critical role of MerTK in mediating efferocytosis, antagonistic ligand-blocking anti-MerTK Abs that inhibit efferocytosis were isolated (see Example 2) with a view to evaluating whether such Abs may enhance the anti-tumor efficacy of agents that upregulate T cell responses, such as anti-PD-1 Abs. An inhibitor of efferocytosis may also synergize with therapies that induce apoptotic responses in the tumor microenvironment, such as certain chemotherapeutic compounds and radiation therapies (Jinushi et al., 2013).

Certain aspects of the disclosed invention encompasses anti-MerTK Abs or antigen-binding portions thereof that inhibit efferocytosis by the MerTK-expressing cell. In certain embodiments, an anti-MerTK Ab or antigen-binding portion thereof of the invention inhibits efferocytosis by a hMerTK-expressing cell with an IC₅₀ of about 5 nM or lower; preferably about 1 nM or lower; or more preferably about 0.1 nM or lower. In certain embodiments, the anti-MerTK Ab inhibits efferocytosis with an IC50 of between about 0.01 nM and about 1 nM. In certain other embodiments, the anti-MerTK Ab inhibits efferocytosis with an IC50 of between about 0.01 nM and about 0.7 nM. In certain preferred embodiments, the anti-MerTK Ab inhibits efferocytosis with an IC₅₀ of between about 0.04 nM and about 0.7 nM. In more preferred embodiments, the anti-MerTK Ab inhibits efferocytosis with an IC₅₀ of between about 0.04 nM and about 0.1 nM. These IC₅₀ values are based on the assay described in Example 2.

Inhibition of MerTK/Ligand Signaling by Anti MerTK mAbs

In certain embodiments, a mAb or antigen-binding portion thereof of the invention inhibits binding of Gas6 to MerTK, for example hMerTK, and inhibits MerTK/Gas6 signaling. In certain embodiments, the anti-MerTK Ab or antigen-binding portion thereof inhibits MerTK/Gas6 signaling with an IC₅₀ of about 50 nM or lower; about 10 nM or lower; about 5 nM or lower; preferably about 1 nM or lower; more preferably about 0.5 nM or lower; even more preferably about 0.1 nM or lower. In certain embodiments, the anti-MerTK Ab inhibits MerTK/Gas6 signaling with an IC₅₀ of between about 0.01 nM and about 10 nM. In certain other embodiments, the anti-MerTK Ab inhibits MerTK/Gas6 signaling with an IC50 of between about 0.05 nM and about 6 nM. In certain preferred embodiments, the anti-MerTK Ab inhibits MerTK/Gas6 signaling with an IC₅₀ of between about 0.08 nM and about 2 nM. In more preferred embodiments, the anti-MerTK Ab inhibits MerTK/Gas6 signaling with an IC₅₀ of between about 0.2 nM and about 2 nM. These IC₅₀ values are based on the assay described in Example 2.

Anti MerTK mAbs that Bind with High Affinity to MerTK

Certain of the anti-MerTK mAbs of this invention bind to MerTK with high affinity. Abs typically bind specifically to their cognate antigen with high affinity, reflected by a dissociation constant (K_{D}) of 1 μM to 10 pM or lower. Any K_{D} greater than about 100 μM is generally considered to indicate nonspecific binding. As used herein, an IgG Ab that “binds specifically” to an antigen refers to an Ab that binds to the antigen and substantially identical antigens with high affinity, which means having a K_{D} of about 100 nM or lower, preferably about 10 nM or lower, more preferably about 5 nM or lower, and even more preferably between about 50 nM and 0.1 nM or lower, but does not bind with high affinity to unrelated antigens. An antigen is “substantially identical” to a given antigen if it exhibits a high degree of sequence identity to the given antigen, for example, if it exhibits at least 80%, at least 90%, preferably at least 95%, more preferably at least 97%, or even more preferably at least 99% sequence identity to the sequence of the given antigen. By way of example, an Ab that binds specifically to hMerTK may also have cross-reactivity with MerTK antigens from certain primate species but may not cross-react with MerTK antigens from certain rodent species or with an antigen other than MerTK, e.g., an Axl or PD-1 antigen.

The term “K_{D},” as used herein, is intended to refer to the dissociation constant for a particular Ab-antigen interaction, which is obtained from the ratio of k_{off} to kₒₙ (i.e., k_{off}/kₒₙ) and is expressed as a molar concentration (e.g., nM). The term “kₒₙ” refers to the association rate or “on rate” for the association of an Ab and its antigen interaction, whereas the term “k_{off}” refers to the dissociation rate for the Ab-antigen complex. K_{D} values for Abs can be determined using methods well established in the art, such as surface plasmon resonance (SPR) or bio-layer interferometry (BLI; ForteBio, Fremont, Calif.). K_{D} values determined by different methods for a single Ab can vary considerably, for example, up to a 1,000-fold. Thus, in comparing the K_{D} values for different Abs, it is important that these K_{D} values be determined using the same method. Where not explicitly stated, and unless the context indicates otherwise, K_{D} values for Ab binding disclosed herein were determined by SPR using a BIACORE® biosensor system (GE Healthcare, Chicago, Ill.).

In certain embodiments of the disclosed invention, the anti-MerTK mAb or antigen-binding portion thereof binds to human MerTK with a K_{D} of: about 100 nM, or about 50 nM, or lower; preferably about 10 nM, or about 5 nM, or lower; more preferably about 1 nM, or about 0.5 nM, or lower; and even more preferably about 0.1 nM, or about 0.05 nM, or lower. In certain embodiments, the anti-MerTK mAb or antigen-binding portion thereof binds to human MerTK with a K_{D} of between about 100 nM and about 0.1 nM. In certain preferred embodiments, the K_{D} is between about 50 nM and about 0.5 nM. In more preferred embodiments, the anti-MerTK mAb or antigen-binding portion thereof binds to human MerTK with a K_{D} of between about 10 nM and about 1 nM. In other more preferred embodiments, the mAb or antigen-binding portion thereof binds to human MerTK with a K_{D} of between about 6 nM and about 2 nM.

In selecting anti-MerTK HuMAbs, hybridomas that bound to hMerTK were screened for cross-reactivity to cMerTK. Accordingly, this disclosure provides anti-MerTK mAbs or antigen-binding portions thereof that bind specifically to cMerTK with high affinity. In certain embodiments, the anti-MerTK mAb or antigen-binding portion thereof binds to cMerTK with a K_{D} of: about 100 nM, or about 50 nM, or lower; preferably about 10 nM, or about 5 nM, or lower; more preferably about 1 nM, or about 0.5 nM, or lower; and even more preferably about 0.1 nM or lower. In certain embodiments, the anti-MerTK mAb or antigen-binding portion thereof binds to cMerTK with a K_{D} of between about 100 nM and about 0.1 nM. In certain preferred embodiments, the K_{D} is between about 50 nM and about 0.5 nM. In more preferred embodiments, the anti-MerTK mAb or antigen-binding portion thereof binds to cMerTK with a K_{D} of between about 10 nM and about 1 nM. In other more preferred embodiments, the mAb or antigen-binding portion thereof binds to cMerTK with a K_{D} of between about 5 nM and about 1 nM.

MAbs that bind specifically to mMerTK were also generated. Accordingly, this disclosure provides mAbs or antigen-binding portions thereof which specifically bind to mMerTK with a K_{D} of: about 100 nM, or about 50 nM, or lower; preferably about 10 nM, or about 5 nM, or lower; more preferably about 1 nM, or about 0.5 nM, or lower; and even more preferably about 0.1 nM or lower. In certain embodiments, the anti-MerTK mAb or antigen-binding portion thereof binds to mMerTK with a K_{D} of between about 100 nM and about 0.1 nM. In certain preferred embodiments, the mAb binds to mMerTK with a K_{D} between about 50 nM and about 0.5 nM. In more preferred embodiments, the anti-MerTK mAb or antigen-binding portion thereof binds to mMerTK with a K_{D} of between about 10 nM and about 1 nM. In other more preferred embodiments, the mAb or antigen-binding portion thereof binds to mMerTK with a K_{D} of between about 5 nM and about 1 nM.

Certain anti-MerTK mAbs disclosed herein, e.g., moMAbs 2D9 and 4 E9, and their humanized versions, 2L105 and 4M60, cross-react with, i.e., bind specifically to all of, m-, h- and cMerTK with high affinity. Other mAbs, e.g., HuMAbs 1B4, 10K11, 22116, 25J60, 25J80, 8N42 and 4K10, cross-react with h- and cMerTK but do not bind to mMerTK. Yet other mAbs, e.g., moMAb 16B9, bind specifically to mMerTK but do not bind to h- and cMerTK. Accordingly, this disclosure provides anti-MerTK mAbs or antigen-binding portions thereof which cross-react with both h- and cMerTK; anti-MerTK mAbs or antigen-binding portions thereof which cross-react with both h- and mMerTK; and anti-MerTK mAbs or antigen-binding portions thereof which cross-react with both h-, c- and mMerTK. In certain embodiments, the anti-MerTK mAb or antigen-binding portion thereof binds specifically to each of h-, c- and mMerTK with a K_{D} of: about 70 nM or lower; preferably between about 50 nM and about 1 nM; and more preferably between about 25 nM and about 3 nM. In certain other embodiments, the anti-MerTK mAb or antigen-binding portion thereof binds specifically to at least both h- and cMerTK with a K_{D} of: about 70 nM or lower; preferably between about 50 nM and about 1 nM; and more preferably between about 25 nM and about 2 nM.

Binning of Anti-MerTK mAbs and Binding of These Abs to Specific Epitopes

Binning experiments with hMerTK identified 3 epitope bins to which the anti-MerTK Abs were assigned. The vast majority of anti-MerTK HuMAbs binned, 11 out of 13, were assigned to Bin 1. Epitope mapping by hydrogen-deuterium exchange mass spectrometry (HDX-MS) and/or yeast display mapped the Bin 1 epitope to the first Ig domain of hMerTK within a linear region spanning approximately amino acids 105 to 165 depending on the specific clone. This disclosure provides a mAb, or an antigen-binding portion thereof, which specifically binds to a Bin 1 epitope on hMerTK. In certain embodiments, the Bin 1 epitope is located in the first Ig domain of hMerTK within a region spanning approximately amino acid residues 105 to 165 as determined by yeast display and/or hydrogen-deuterium exchange mass spectrometry (HDX-MS) epitope mapping. In certain other embodiments, the Bin 1 epitope is located within a region of hMerTK spanning approximately amino acid residues 126 to 155 as determined by HDX-MS epitope mapping. In further embodiments, the Bin 1 epitope comprises at least one, two, three, four, five, six, seven, ten, twenty or all of the amino acid residues 126 to 155 as determined by HDX-MS epitope mapping.

One of the anti-MerTK HuMAbs binned, 25B10, was assigned to Bin 2. Following optimization of anti-hMerTK HuMAbs to mitigate sequence liabilities, enhance binding affinities, and revert to germline amino acids (Example 2), multiple mAbs were derived from mAb 25B10, of which mAbs 25J60 and 25J80 are included in Tables 1 and 2. MoMAbs 2D9 and 4E9, and their humanized variants, 2L105 and 4M60, respectively, were also assigned to Bin 2. Epitope mapping by HDX-MS and/or yeast display mapped the Bin 2 epitope to the second Ig domain of hMerTK within a linear region spanning approximately amino acids 195 to 270 depending on the specific clone.

The disclosed invention encompasses an isolated Ab, preferably a mAb, or an antigen-binding portion thereof, which specifically binds to a Bin 2 epitope on hMerTK. In certain embodiments, the Bin 2 epitope is located in the second Ig domain of hMerTK within a region spanning approximately amino acid residues 195 to 270 as determined by yeast display and/or HDX-MS epitope mapping. In certain other embodiments, the Bin 2 epitope is located within a region of hMerTK spanning approximately amino acid residues 231 to 249 (²³¹WVQNSSRVNEQPEKSPSVL²⁴⁹) as determined by HDX-MS epitope mapping. In further embodiments, the Bin 2 epitope comprises one, two, three, four, five, six or all of the amino acid residues N234, 5236, R237, E240, Q241, P242 and G269 as determined by yeast display epitope mapping. In certain preferred embodiments, the Bin2 epitope comprises the amino acid residues N234, S236, R237, E240, Q241, P242 and G269. In other embodiments, the Bin 2 epitope comprises at least one, two, three, four, five, six, seven, ten or all of the amino acid residues 231 to 249 and amino acid residue G269 as determined by HDX-MS and yeast display epitope mapping.

Both Bin 1 and Bin 2 epitope regions are consistent with ligand blockade based on homology modeling of the Gas6/Axl crystal structure. However, the results of preliminary toxicology studies in cynomolgus monkeys using representative mAbs binding to the Bin 1 or Bin2 epitopes showed that two different mAbs that bind to the Bin 1 epitope cause severe adverse effects, specifically peripheral neuropathy, in the monkeys whereas mAbs that bind to the Bin 2 epitope are well tolerated. Accordingly, an anti-MerTK mAb that binds to a Bin2 epitope appears to be preferable for therapeutic uses. In preferred embodiments, the anti-MerTK mAb binds to a Bin2 epitope.

A single anti-MerTK HuMAbs binned was assigned to Bin 3. This disclosure provides an isolated Ab, preferably a mAb, or an antigen-binding portion thereof, which specifically binds to a Bin 3 epitope on hMerTK. In certain embodiments, the Bin 3 epitope is located in the Fn domains of hMerTK within a region spanning approximately amino acid residues 420 to 490 as determined by yeast display and/or HDX-MS epitope mapping.

Anti MerTK mAbs that Cross-Compete with a Reference Ab for Binding to MerTK

Also encompassed within the scope of the disclosed invention is an isolated Ab, preferably a mAb, or an antigen-binding portion thereof, which specifically binds to hMerTK expressed on the surface of a cell, and cross-competes with a reference Ab or a reference antigen-binding portion thereof for binding to hMerTK. The ability of a pair of Abs to “cross-compete” for binding to an antigen, e.g., MerTK, indicates that a first Ab binds to substantially the same epitope region of the antigen as, and sterically hinders the binding of, a second Ab to that particular epitope region and, conversely, the second Ab binds to substantially the same epitope region of the antigen as, and sterically hinders the binding of, the first Ab to that epitope region. Thus, the ability of a test Ab to competitively inhibit the binding of, for example, mAb 2L105 to hMerTK, demonstrates that the test Ab binds to substantially the same epitope region of human PD-1 as does mAb 2L105.

A first Ab is considered to bind to “substantially the same epitope” as does a second Ab if the first Ab reduces the binding of the second Ab to an antigen by at least about 40%. Preferably, the first Ab reduces the binding of the second Ab to the antigen by more than about 50% (e.g., at least about 60% or at least about 70%). In more preferred embodiments, the first Ab reduces the binding of the second Ab to the antigen by more than about 70% (e.g., at least about 80%, at least about 90%, or about 100%). The order of the first and second Abs can be reversed, i.e. the “second” Ab can be first bound to the surface and the “first” is thereafter brought into contact with the surface in the presence of the “second” Ab. The Abs are considered to “cross-compete” if a competitive reduction in binding to the antigen is observed irrespective of the order in which the Abs are added to the immobilized antigen.

Cross-competing Abs are expected to have functional properties very similar to the properties of the reference Abs by virtue of their binding to substantially the same epitope region of an antigen such as a MerTK receptor. The higher the degree of cross-competition, the more similar will the functional properties be. For example, two cross-competing Abs are expected to have essentially the same functional properties if they each inhibit binding of the other to an epitope by at least about 80%. This similarity in function is expected to be even closer if the cross-competing Abs exhibit similar affinities for binding to the epitope as measured by the dissociation constant (K_{D}).

Cross-competing anti-antigen Abs can be readily identified based on their ability to detectably compete in standard antigen binding assays, including BIACORE® analysis, ELISA assays or flow cytometry, using either recombinant antigen molecules or cell-surface expressed antigen molecules. By way of example, a simple competition assay to identify whether a test Ab competes with HuMAb 25J80 for binding to human MerTK may involve: (1) measuring the binding of 25J80, applied at saturating concentration, to a BIACORE® chip (or other suitable medium for SPR analysis) onto which human MerTK is immobilized, and (2) measuring the binding of 25J80 to a human MerTK-coated BIACORE® chip (or other medium suitable) to which the test Ab has been previously bound. The binding of 25J80 to the MerTK-1-coated surface in the presence and absence of the test Ab is compared. A significant (e.g., more than about 40%) reduction in binding of 25J80 in the presence of the test Ab indicates that both Abs recognize substantially the same epitope such that they compete for binding to the MerTK target. The percentage by which the binding of a first Ab to an antigen is inhibited by a second Ab can be calculated as: [1-(detected binding of first Ab in presence of second Ab)/(detected binding of first Ab in absence of second Ab)]×100. To determine whether the Abs cross-compete, the competitive binding assay is repeated except that the binding of the test Ab to the MerTK-coated chip in the presence of 25J80 is measured.

Any of the anti-MerTK Abs disclosed herein may serve as a reference Ab in cross-competition assays. In certain embodiments, the reference Ab comprises:

(a) a V_{H} comprising consecutively linked amino acids having the sequence set forth as SEQ ID NO: 217, 221, 225, 229, 233, 237, 241, 245, 249, 253, 255, or 257; and

(b) a V_{L} comprising consecutively linked amino acids having the sequence set forth as SEQ ID NO: 218, 222, 226, 230, 234, 238, 242, 246, 250, 254, 256, or 258.

In further embodiments, the reference Ab or reference antigen-binding portion thereof comprises:

(a) a V_{H} comprising consecutively linked amino acids having the sequence set forth as SEQ ID NO: 217 and a V_{L} comprising consecutively linked amino acids having the sequence set forth as SEQ ID NO: 218;

(b) a V_{H} comprising consecutively linked amino acids having the sequence set forth as SEQ ID NO: 221 and a V_{L} comprising consecutively linked amino acids having the sequence set forth as SEQ ID NO: 222;

(c) a V_{H} comprising consecutively linked amino acids having the sequence set forth as SEQ ID NO: 225 and a V_{L} comprising consecutively linked amino acids having the sequence set forth as SEQ ID NO: 226;

(d) a V_{H} comprising consecutively linked amino acids having the sequence set forth as SEQ ID NO: 229 and a V_{L} comprising consecutively linked amino acids having the sequence set forth as SEQ ID NO: 230;

(e) a V_{H} comprising consecutively linked amino acids having the sequence set forth as SEQ ID NO: 233 and a V_{L} comprising consecutively linked amino acids having the sequence set forth as SEQ ID NO: 234;

(f) a V_{H} comprising consecutively linked amino acids having the sequence set forth as SEQ ID NO: 237 and a V_{L} comprising consecutively linked amino acids having the sequence set forth as SEQ ID NO: 238;

(g) a V_{H} comprising consecutively linked amino acids having the sequence set forth as SEQ ID NO: 241 and a V_{L} comprising consecutively linked amino acids having the sequence set forth as SEQ ID NO: 242;

(h) a V_{H} comprising consecutively linked amino acids having the sequence set forth as SEQ ID NO: 245 and a V_{L} comprising consecutively linked amino acids having the sequence set forth as SEQ ID NO: 246;

(i) a V_{H} comprising consecutively linked amino acids having the sequence set forth as SEQ ID NO: 249 and a V_{L} comprising consecutively linked amino acids having the sequence set forth as SEQ ID NO: 250;

(j) a V_{H} comprising consecutively linked amino acids having the sequence set forth as SEQ ID NO: 253 and a V_{L} comprising consecutively linked amino acids having the sequence set forth as SEQ ID NO: 254;

(k) a V_{H} comprising consecutively linked amino acids having the sequence set forth as SEQ ID NO: 255 and a V_{L} comprising consecutively linked amino acids having the sequence set forth as SEQ ID NO: 256; or

(l) a V_{H} comprising consecutively linked amino acids having the sequence set forth as SEQ ID NO: 257 and a V_{L} comprising consecutively linked amino acids having the sequence set forth as SEQ ID NO: 258.

Structurally Defined Anti-MerTK mAbs

The present disclosure also provides an isolated Ab, preferably a mAb, or an antigen-binding portion thereof, which specifically binds to hMerTK expressed on the surface of a cell, and comprises the CDR1, CDR2 and CDR3 domains in each of:

(a) a V_{H} comprising consecutively linked amino acids having the sequence set forth as SEQ ID NO: 217 and a V_{L} comprising consecutively linked amino acids having the sequence set forth as SEQ ID NO: 218;

(b) a V_{H} comprising consecutively linked amino acids having the sequence set forth as SEQ ID NO: 221 and a V_{L} comprising consecutively linked amino acids having the sequence set forth as SEQ ID NO: 222;

(c) a V_{H} comprising consecutively linked amino acids having the sequence set forth as SEQ ID NO: 225 and a V_{L} comprising consecutively linked amino acids having the sequence set forth as SEQ ID NO: 226;

(d) a V_{H} comprising consecutively linked amino acids having the sequence set forth as SEQ ID NO: 229 and a V_{L} comprising consecutively linked amino acids having the sequence set forth as SEQ ID NO: 230;

(e) a V_{H} comprising consecutively linked amino acids having the sequence set forth as SEQ ID NO: 233 and a V_{L} comprising consecutively linked amino acids having the sequence set forth as SEQ ID NO: 234;

(f) a V_{H} comprising consecutively linked amino acids having the sequence set forth as SEQ ID NO: 237 and a V_{L} comprising consecutively linked amino acids having the sequence set forth as SEQ ID NO: 238;

(g) a V_{H} comprising consecutively linked amino acids having the sequence set forth as SEQ ID NO: 241 and a V_{L} comprising consecutively linked amino acids having the sequence set forth as SEQ ID NO: 242;

(h) a V_{H} comprising consecutively linked amino acids having the sequence set forth as SEQ ID NO: 245 and a V_{L} comprising consecutively linked amino acids having the sequence set forth as SEQ ID NO: 246;

(i) a V_{H} comprising consecutively linked amino acids having the sequence set forth as SEQ ID NO: 249 and a V_{L} comprising consecutively linked amino acids having the sequence set forth as SEQ ID NO: 250;

(j) a V_{H} comprising consecutively linked amino acids having the sequence set forth as SEQ ID NO: 253 and a V_{L} comprising consecutively linked amino acids having the sequence set forth as SEQ ID NO: 254;

(k) a V_{H} comprising consecutively linked amino acids having the sequence set forth as SEQ ID NO: 255 and a V_{L} comprising consecutively linked amino acids having the sequence set forth as SEQ ID NO: 256; or

(l) a V_{H} comprising consecutively linked amino acids having the sequence set forth as SEQ ID NO: 257 and a V_{L} comprising consecutively linked amino acids having the sequence set forth as SEQ ID NO: 258.

Different methods have been developed to delineate the CDR domains within an Ab. In addition to the widely used Kabat definition, others including the Chothia, AbNum, AbM, contact and IMGT definitions that seek to address deficiencies of the Kabat definitions, have been employed.

The approach of Kabat and co-workers (Wu and Kabat, 1970; Kabat et al., 1983), was based on the assumption that CDRs include the most variable positions in Abs and therefore could be identified by aligning the fairly limited number of Ab sequences then available. Based on this alignment, Kabat et al. introduced a numbering scheme for the residues in the hypervariable regions and determined which positions mark the beginning and the end of each CDR (http://bioinforg.uk/abs/simkab.html).

The Chothia definition is based on the analysis of a small number of Ab structures to determine the relationship between the sequences of the Abs and the structural loop regions of their CDRs (Chothia et al., 1987; 1989; Al-Lazikani et al., 1997; http://bioinforg.uk/abs/chothia.html). The boundaries of the FRs and the CDRs were determined and the latter have been shown to adopt a restricted set of conformations based on the presence of certain residues at key positions in the CDRs and the flanking FRs. The resulting Chothia numbering scheme is almost identical to the Kabat scheme but, based on structural considerations, places insertions in the V_{L} CDR1 and V_{H} CDR1 at different positions. As more experimental data became available, there has been an ongoing re-analysis and re-definition of the boundaries of the CDRs. Abhinandan and Martin (2008) analyzed Ab sequence alignments in the context of structure and found that approximately 10% of the sequences in the manually annotated Kabat database contain errors or inconsistencies. They proposed a corrected version of the Chothia scheme which is structurally correct throughout the CDRs and frameworks, and developed a software tool (AbNum; available at http://www.bioinf org.uk/abs/abnum/) that applies the Kabat, Chothia and modified-Chothia numbering in an automatic and reliable manner. Another method, the AbM definition, represents a compromise between the Kabat and Chothia definitions and is used by Oxford Molecular Group's AbM Ab modelling software (http://www.bioinforg.uk/abs; Martin et al., 1989).

The contact definition is based on an analysis of the contacts between Ab and antigen in the complex crystal structures available in the Protein Data Bank (http://bioinf.org.uk/abs/; MacCallum et al., 1996).

A more recent attempt to define CDRs is that of the IMGT database (Lefranc et al. (2003; http://www.imgt.org) which curates nucleotide sequence information for Ig's, T-cell receptors (TcR) and Major Histocompatibility Complex (MHC) molecules. It proposes a uniform numbering system for Ig and TcR sequences, based on aligning more than 5000 Ig and TcR variable region sequences.

CDRs for the disclosed anti-MerTK mAbs disclosed herein have been delineated using the Kabat, Chothia and IMGT definitions (see Tables 3-14). For any given mAb, a CDR may be identified using any of the Kabat, Chothia and IMGT definitions as shown in Tables 3-14, and any combination thereof. Accordingly, the disclosure provides isolated Abs, preferably mAbs, comprising sets of six CDRs corresponding to CDR sequences shown in Tables 3-14.

By way of example, based on the mAb 1B4, the disclosure provides an isolated Ab, preferably a mAb, or an antigen-binding portion thereof, which comprises the following CDR domains as defined by the Kabat, Chothia and/or IMGT methods:

(a) a heavy chain variable region CDR1 comprising consecutively linked amino acids having the sequence set forth as any one of SEQ ID Nos. 1-3;

(b) a heavy chain variable region CDR2 comprising consecutively linked amino acids having the sequence set forth as any one of SEQ ID Nos. 4-6;

(c) a heavy chain variable region CDR3 comprising consecutively linked amino acids having the sequence set forth as any one of SEQ ID Nos. 7-9;

(d) a light chain variable region CDR1 comprising consecutively linked amino acids having the sequence set forth as any one of SEQ ID Nos. 10-12;

(e) a light chain variable region CDR2 comprising consecutively linked amino acids having the sequence set forth as any one of SEQ ID Nos. 13-15; and

(f) a light chain variable region CDR3 comprising consecutively linked amino acids having the sequence set forth as any one of SEQ ID Nos. 16-18.

The disclosure also provides an isolated Ab, preferably a mAb, or an antigen-binding portion thereof, which comprises the following CDR domains as defined by the IMGT method:

(a) a heavy chain variable region CDR1 comprising consecutively linked amino acids having the sequence set forth as SEQ ID NO:3;

(b) a heavy chain variable region CDR2 comprising consecutively linked amino acids having the sequence set forth as SEQ ID NO:6;

(c) a heavy chain variable region CDR3 comprising consecutively linked amino acids having the sequence set forth as SEQ ID NO:9;

(d) a light chain variable region CDR1 comprising consecutively linked amino acids having the sequence set forth as SEQ ID NO:12;

(e) a light chain variable region CDR2 comprising consecutively linked amino acids having the sequence set forth as any one of SEQ ID NO:15; and

(f) a light chain variable region CDR3 comprising consecutively linked amino acids having the sequence set forth as any one of SEQ ID NO:18.

As another example, based on the mAb 25J80, the disclosure provides an isolated Ab, preferably a mAb, or an antigen-binding portion thereof, which comprises the following CDR domains as defined by the Kabat, Chothia and/or IMGT methods:

(a) a heavy chain variable region CDR1 comprising consecutively linked amino acids having the sequence set forth as any one of SEQ ID Nos. 73-75;

(b) a heavy chain variable region CDR2 comprising consecutively linked amino acids having the sequence set forth as any one of SEQ ID Nos. 76-78;

(c) a heavy chain variable region CDR3 comprising consecutively linked amino acids having the sequence set forth as any one of SEQ ID Nos. 79-81;

(d) a light chain variable region CDR1 comprising consecutively linked amino acids having the sequence set forth as any one of SEQ ID Nos. 82-84;

(e) a light chain variable region CDR2 comprising consecutively linked amino acids having the sequence set forth as any one of SEQ ID Nos. 85-87; and

(f) a light chain variable region CDR3 comprising consecutively linked amino acids having the sequence set forth as any one of SEQ ID Nos. 88-90.

The disclosure also provides an isolated Ab, preferably a mAb, or an antigen-binding portion thereof, which comprises the following CDR domains as defined by the Kabat method:

(a) a heavy chain variable region CDR1 comprising consecutively linked amino acids having the sequence set forth in SEQ ID NO:73;

(b) a heavy chain variable region CDR2 comprising consecutively linked amino acids having the sequence set forth as SEQ ID NO: 76;

(c) a heavy chain variable region CDR3 comprising consecutively linked amino acids having the sequence set forth as SEQ ID NO:79;

(d) a light chain variable region CDR1 comprising consecutively linked amino acids having the sequence set forth as SEQ ID NO:82;

(e) a light chain variable region CDR2 comprising consecutively linked amino acids having the sequence set forth as SEQ ID NO:85; and

(f) a light chain variable region CDR3 comprising consecutively linked amino acids having the sequence set forth as SEQ ID NO:88.

The Kabat definition is the most commonly used method to predict CDR domains, notwithstanding it was developed when no structural information on Abs was available. Where not explicitly stated, and unless the context indicates otherwise, CDRs disclosed herein have been identified using the Kabat definition.

The disclosed invention also encompasses an isolated Ab, preferably a mAb, or an antigen-binding portion thereof, which specifically binds to hMerTK expressed on the surface of a cell, wherein the isolated Ab or antigen-binding portion thereof comprises:

(a) a V_{H} comprising consecutively linked amino acids having the sequence set forth as SEQ ID NO: 217 and a V_{L} comprising consecutively linked amino acids having the sequence set forth as SEQ ID NO: 218;

(b) a V_{H} comprising consecutively linked amino acids having the sequence set forth as SEQ ID NO: 221 and a V_{L} comprising consecutively linked amino acids having the sequence set forth as SEQ ID NO: 222;

(c) a V_{H} comprising consecutively linked amino acids having the sequence set forth as SEQ ID NO: 225 and a V_{L} comprising consecutively linked amino acids having the sequence set forth as SEQ ID NO: 226;

(d) a V_{H} comprising consecutively linked amino acids having the sequence set forth as SEQ ID NO: 229 and a V_{L} comprising consecutively linked amino acids having the sequence set forth as SEQ ID NO: 230;

(e) a V_{H} comprising consecutively linked amino acids having the sequence set forth as SEQ ID NO: 233 and a V_{L} comprising consecutively linked amino acids having the sequence set forth as SEQ ID NO: 234;

(f) a V_{H} comprising consecutively linked amino acids having the sequence set forth as SEQ ID NO: 237 and a V_{L} comprising consecutively linked amino acids having the sequence set forth as SEQ ID NO: 238;

(g) a V_{H} comprising consecutively linked amino acids having the sequence set forth as SEQ ID NO: 241 and a V_{L} comprising consecutively linked amino acids having the sequence set forth as SEQ ID NO: 242;

(h) a V_{H} comprising consecutively linked amino acids having the sequence set forth as SEQ ID NO: 245 and a V_{L} comprising consecutively linked amino acids having the sequence set forth as SEQ ID NO: 246;

(i) a V_{H} comprising consecutively linked amino acids having the sequence set forth as SEQ ID NO: 249 and a V_{L} comprising consecutively linked amino acids having the sequence set forth as SEQ ID NO: 250;

(j) a V_{H} comprising consecutively linked amino acids having the sequence set forth as SEQ ID NO: 253 and a V_{L} comprising consecutively linked amino acids having the sequence set forth as SEQ ID NO: 254;

(k) a V_{H} comprising consecutively linked amino acids having the sequence set forth as SEQ ID NO: 255 and a V_{L} comprising consecutively linked amino acids having the sequence set forth as SEQ ID NO: 256; or

(l) a V_{H} comprising consecutively linked amino acids having the sequence set forth as SEQ ID NO: 257 and a V_{L} comprising consecutively linked amino acids having the sequence set forth as SEQ ID NO: 258.

The invention further encompasses an isolated Ab, preferably a mAb, or an antigen-binding portion thereof, which specifically binds to hMerTK expressed on the surface of a cell, wherein the isolated Ab or antigen-binding portion thereof comprises:

(a) a heavy chain comprising consecutively linked amino acids having the sequence set forth as SEQ ID NO: 219 and a light chain comprising consecutively linked amino acids having the sequence set forth as SEQ ID NO: 220;

(b) a heavy chain comprising consecutively linked amino acids having the sequence set forth as SEQ ID NO: 223 and a light chain comprising consecutively linked amino acids having the sequence set forth as SEQ ID NO: 224;

(c) a heavy chain comprising consecutively linked amino acids having the sequence set forth as SEQ ID NO: 227 and a light chain comprising consecutively linked amino acids having the sequence set forth as SEQ ID NO: 228;

(d) a heavy chain comprising consecutively linked amino acids having the sequence set forth as SEQ ID NO: 231 and a light chain comprising consecutively linked amino acids having the sequence set forth as SEQ ID NO: 232;

(e) a heavy chain comprising consecutively linked amino acids having the sequence set forth as SEQ ID NO: 235 and a light chain comprising consecutively linked amino acids having the sequence set forth as SEQ ID NO: 236;

(f) a heavy chain comprising consecutively linked amino acids having the sequence set forth as SEQ ID NO: 239 and a light chain comprising consecutively linked amino acids having the sequence set forth as SEQ ID NO: 240;

(g) a heavy chain comprising consecutively linked amino acids having the sequence set forth as SEQ ID NO: 243 and a light chain comprising consecutively linked amino acids having the sequence set forth as SEQ ID NO: 244;

(h) a heavy chain comprising consecutively linked amino acids having the sequence set forth as SEQ ID NO: 247 and a light chain comprising consecutively linked amino acids having the sequence set forth as SEQ ID NO: 248; or

(i) a heavy chain comprising consecutively linked amino acids having the sequence set forth as SEQ ID NO: 251 and a light chain comprising consecutively linked amino acids having the sequence set forth as SEQ ID NO: 252.

Anti-MerTK Abs comprising V_{H} and V_{L} regions having amino acid sequences that are highly similar or homologous to the amino acid sequences of any of the above anti-MerTK Abs and which retain the functional properties of these Abs are also suitable for use in the present methods. For example, suitable Abs include mAbs comprising a V_{H} and V_{L} region each comprising consecutively linked amino acids having a sequence that is at least 80% identical to the amino acid sequence set forth in SEQ ID Nos. 245 and/or 246, respectively. In further embodiments, for example, the V_{H} and/or V_{L} amino acid sequences exhibits at least 85%, 90%, 95%, or 99% identity to the sequences set forth in SEQ ID Nos. 245 and/or 246, respectively. As used herein, the percent sequence identity between two amino acid sequences is a function of the number of identical positions shared by the sequences relative to the length of the sequences compared (i.e., % identity=number of identical positions/total number of positions being compared×100), taking into account the number of any gaps, and the length of each such gap, introduced to maximize the degree of sequence identity between the two sequences. The comparison of sequences and determination of percent identity between two sequences can be accomplished using mathematical algorithms that are well known to those of ordinary skill in the art.

In certain embodiments, the isolated anti-MerTK Ab or antigen-binding portion thereof comprises a heavy chain constant region which is of a human IgG1, IgG2, IgG3 or IgG4 isotype. In certain preferred embodiments, the heavy chain constant region is of a human IgG4 isotype. In other preferred embodiments, the isolated anti-MerTK Ab or antigen-binding portion thereof is of a human IgG1 isotype. In certain embodiments, the isolated anti-MerTK Ab is a full-length Ab of an IgG1, IgG2, IgG3 or IgG4 isotype. In further embodiments, the full-length Ab is of an IgG1 or IgG4 isotype.

Functional Antigen-Binding Portions of Anti-MerTK Abs

Anti-MerTK Abs provided by the disclosure also include antigen-binding fragments in addition to full-length Abs. It has been amply demonstrated that the antigen-binding function of an Ab can be performed by fragments of a full-length Ab. Examples of binding fragments encompassed within the term “antigen-binding portion” of an Ab include (i) a Fab fragment, a monovalent fragment consisting of the V_{L}, V_{H}, C_{L} and C_{H1} domains; (ii) a F(ab′)₂ fragment, a bivalent fragment consisting of two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an Ab; and (v) a single-domain Ab (sdAb) or nanobody, consisting of a single monomeric variable domain of an Ab. In addition to conventional Abs, camelid species such as camels, alpacas and llamas, and cartilaginous fish such as sharks and rays contain a subset of heavy chain Abs (hcAbs) consisting of heavy chain homodimers comprising three CDRs and lacking light chains. The first sdAbs were originally engineered from the hcAbs found in camelids (these are called VIM fragments) or in cartilaginous fish (VNAR fragments), but can also be generated by splitting the dimeric variable domains from conventional Abs. In addition to sdAbs derived from heavy chain variable domains, nanobodies derived from light chains have also been shown to bind selectively to specific antigens.

Ab fragments, obtained initially through proteolysis with enzymes such as papain and pepsin, have been subsequently engineered into monovalent and multivalent antigen-binding fragments. For example, although the two domains of the Fv fragment, V_{L} and V_{H}, are coded for by separate genes, they can be joined, using recombinant methods, by a synthetic linker peptide that enables them to be made as a single protein chain in which the V_{L} and V_{H} regions pair to form monovalent molecules known as single chain variable fragments (scFv). Divalent or bivalent scFv's (di-scFv's or bi-scFv's) can be engineered by linking two scFv's in within a single peptide chain known as a tandem scFv which contains two V_{H} and two V_{L} regions. ScFv dimers and higher multimers can also be created using linker peptides of fewer than 10 amino acids that are too short for the two variable regions to fold together, which forces the scFv's to dimerize and produce diabodies or form other multimers. Diabodies have been shown to bind to their cognate antigen with much higher affinity than the corresponding scFv's, having dissociation constants up to 40-fold lower than the K_{D} values for the scFv's. Very short linkers (<3 amino acids) lead to the formation of trivalent triabodies or tetravalent tetrabodies that exhibit even higher affinities for to their antigens than diabodies. Other variants include minibodies, which are scFv-C_{H3} dimers, and larger scFv-Fc fragments (scFv-C_{H2}-C_{H3} dimers), and even an isolated CDR may exhibit antigen-binding function. These Ab fragments are engineered using conventional recombinant techniques known to those of skill in the art, and the fragments are screened for utility in the same manner as are intact Abs. All of the above proteolytic and engineered fragments of Abs and related variants (see Hollinger and Hudson, 2005; Olafsen and Wu, 2010, for further details) are intended to be encompassed within the term “antigen-binding portion” of an Ab.

In certain aspects of the disclosed invention, the antigen-binding portion of an isolated anti-MerTK Ab is an Ab fragment or a single chain Ab. In certain embodiments, the Ab fragment is selected from a Fab, F(ab′)₂, Fd and Fv fragment, a sdAb, a single-chain variable fragment (scFv), a divalent scFv (di-scFv) and bivalent scFv (bi-scFv), a diabody, a minibody, and a CDR. In certain preferred embodiments, the Ab fragment is selected from a Fab, F(ab′)₂, Fd and Fv fragment and a single chain variable fragment (scFv).

In certain embodiments, the isolated anti-MERTK Ab or antigen-binding portion thereof is a human Ab or fragment thereof. In other embodiments, it is a humanized Ab or fragment thereof. In further embodiments, it is a chimeric Ab or fragment thereof. In other embodiments, the isolated anti-MERTK Ab or antigen-binding portion thereof is a mouse Ab or fragment thereof. For administration to human subjects, the Abs are preferably chimeric Abs or, more preferably, humanized or human Abs. Such chimeric, humanized, human or mouse mAbs can be prepared and isolated by methods well known in the art.

### Anti-MerTK Immunoconjugates

In another aspect, the present invention relates to any one of the isolated anti-MerTK Abs disclosed herein, or an antigen-binding portion thereof, linked to a therapeutic agent, such as a cytotoxin or a radioactive isotope. Such conjugates are referred to herein as “immunoconjugates”. Cytotoxins can be conjugated to Abs of the invention using linker technology available in the art. Methods for preparing radioimmunoconjugates are also established in the art.

### Bispecific Molecules

In another aspect, the present invention relates to bispecific molecules comprising any one of the isolated anti-MerTK Abs disclosed herein, or an antigen-binding portion thereof, linked to a binding domain that has a different binding specificity than the anti-MerTK mAb or antigen-binding portion thereof. The binding domain may be a functional molecule, e.g., another Ab, antigen-binding portion of an Ab, or a ligand for a receptor), such that the bispecific molecule generated binds to at least two different binding sites or target molecules.

### Nucleic Acids Encoding Anti-MerTK Abs and Use for Expressing Abs

Another aspect of the disclosure pertains to nucleic acids that encode the isolated anti-MerTK Abs of the invention. The disclosure provides an isolated nucleic acid encoding any of the MerTK Abs or antigen-binding portions thereof described herein. An “isolated” nucleic acid refers to a nucleic acid composition of matter that is markedly different, i.e., has a distinctive chemical identity, nature and utility, from nucleic acids as they exist in nature. For example, an isolated DNA, unlike native DNA, is a free-standing portion of a native DNA and not an integral part of a larger structural complex, the chromosome, found in nature. Further, an isolated DNA, unlike native DNA, can be used as a PCR primer or a hybridization probe for, among other things, measuring gene expression and detecting biomarker genes or mutations for diagnosing disease or predicting the efficacy of a therapeutic. An isolated nucleic acid may also be purified so as to be substantially free of other cellular components or other contaminants, e.g., other cellular nucleic acids or proteins, using standard techniques well known in the art.

Nucleic acids of the invention can be obtained using standard molecular biology techniques. For Abs expressed by hybridomas (e.g., hybridomas prepared from transgenic mice carrying human Ig genes as described in Example 1), cDNAs encoding the light and heavy chains or variable regions of the Ab made by the hybridoma can be obtained by standard PCR amplification techniques. Once DNA fragments encoding V_{H} and V_{L} segments are obtained, these DNA fragments can be further manipulated using standard recombinant DNA techniques, for example, to convert the variable region DNAs to full-length Ab chain genes, to Fab fragment genes, or to a scFv gene. For Abs obtained from an Ig gene library (e.g., using phage display techniques), nucleic acids encoding the Ab can be recovered from the library.

A nucleic acid of the invention can be, for example, RNA or DNA such as cDNA or genomic DNA. In preferred embodiments, the nucleic acid is a cDNA.

The disclosure also provides an expression vector comprising an isolated nucleic which encodes an anti-MerTK Ab or antigen-binding portion thereof. The disclosure further provides a host cell comprising said expression vector. Eukaryotic cells, and most preferably mammalian host cells, are preferred as host cells for expressing Abs because such eukaryotic cells, and in particular mammalian cells, are more likely than prokaryotic cells to assemble and secrete a properly folded and immunologically active Ab. Preferred mammalian host cells for expressing the recombinant Abs of the invention include Chinese Hamster Ovary (CHO) cells (Kaufman and Sharp, 1982), NSO myeloma cells, COS cells and SP2 cells.

The host cell may be used in a method for preparing an anti-MerTK mAb or an antigen-binding portion thereof, which method comprises expressing the mAb or antigen-binding portion thereof in the host cell and isolating the mAb or antigen-binding portion thereof from the host cell. The host cell may be used ex vivo or in vivo. The DNAs encoding the Ab heavy and light chains can be inserted into separate expression vectors or, more typically, are both inserted into the same vector. The V_{H} and V_{L} segments of an Ab can be used to create full-length Abs of any isotype by inserting DNAs encoding these variable regions into expression vectors already encoding heavy chain and light chain constant regions of the desired isotype such that the V_{H} segment is operatively linked to the CH segment(s) within the vector and the V_{K} segment is operatively linked to the C_{L} segment within the vector.

Another aspect of this invention relates to a transgenic mouse comprising human Ig heavy and light chain transgenes, wherein the mouse expresses any of the anti-MerTK HuMAbs disclosed herein. The invention also encompasses a hybridoma prepared from said mouse, wherein the hybridoma produces the HuMAb.

### Anti-MerTK Abs Suitable for Use in the Disclosed Therapeutic Methods

Anti-MerTK Abs suitable for use in the disclosed methods are isolated Abs, preferably mAbs or antigen-binding portions thereof, that bind specifically to MerTK expressed on the surface of a cell with high specificity and affinity. In certain preferred embodiments, the anti-MerTK Ab cross-reacts with both hMerTK and cMerTK, which facilitates toxicological studies of the Ab in cynomolgus monkeys. In certain embodiments, the anti-MerTK Ab cross-reacts with hMerTK, cMerTK and mMerTK. In certain embodiments, the anti-MerTK Ab or antigen-binding portion thereof inhibits the binding of Gas6 to MerTK and inhibit MerTK/Gas6 signaling. In certain preferred embodiments, the anti-MerTK Ab or antigen-binding portion thereof inhibits efferocytosis by the MerTK-expressing cell. In certain embodiments, the anti-MerTK Ab or antigen-binding portion thereof binds to an epitope of hMerTK located within the region spanning approximately amino acids 105 to 165, an epitope located within the region spanning approximately amino acids 195 to 270, or an epitope located within the region spanning approximately amino acids 420 to 490. In certain preferred embodiments, the anti-MerTK Ab or antigen-binding portion thereof binds to an epitope of hMerTK located within the region spanning approximately amino acids 195 to 270, or more specifically within the region spanning approximately amino acids 231 to 249. In other preferred embodiments, the anti-MerTK Ab or antigen-binding portion thereof binds to an epitope of hMerTK comprising at least one, two, three, four, five six or all of residues N234, 5236, 8237, E240, Q241, P242 and G269. In yet other preferred embodiments, the anti-MerTK Ab or antigen-binding portion thereof interacts synergistically with a checkpoint inhibitor, such as an anti-PD-1/anti-PD-L1 Ab, in reducing the growth of cancer cells in vivo. Abs are considered herein to interact synergistically if the anti-tumor efficacy of the combination of these Abs is greater than the sum of the anti-tumor efficacy exhibited by each Ab individually.

Although the efficacy of combination therapy with an anti-MerTK Ab and a checkpoint inhibitor have been demonstrated herein primarily using an anti-PD-1 Ab, several other costimulatory and inhibitory receptors and ligands that regulate T cell responses have been identified. Examples of stimulatory receptors include Inducible T cell Co-Stimulator (ICOS), CD137 (4-1BB), CD134 (OX40), CD27, Glucocorticoid-Induced TNFR-Related protein (GITR), and HerpesVirus Entry Mediator (HVEM), whereas examples of inhibitory receptors in addition to PD-1/PD-L1 include Cytotoxic T-Lymphocyte-Associated protein 4 (CTLA-4), B and T Lymphocyte Attenuator (BTLA), T cell Immunoglobulin and Mucin domain-3 (TIM-3), Lymphocyte Activation Gene-3 (LAG-3), Killer Immunoglobulin-like Receptor (KIR), adenosine A2a receptor (A2aR), Killer cell Lectin-like Receptor G1 (KLRG-1), Natural Killer Cell Receptor 2B4 (CD244), CD160, T cell Immunoreceptor with Ig and ITIM domains (TIGIT), and the receptor for V-domain Ig Suppressor of T cell Activation (VISTA), (Mellman et al., 2011; Pardoll, 2012; Baitsch et al., 2012). These receptors and their ligands provide targets for therapeutics designed to stimulate, or prevent the suppression, of an immune response so as to thereby attack tumor cells (Weber, 2010; Mellman et al., 2011; Pardoll, 2012). Stimulatory receptors or receptor ligands are targeted by agonist agents, whereas inhibitory receptors or receptor ligands are targeted by blocking agents. Because many of the immune checkpoints are initiated by ligand-receptor interactions, they can be readily blocked by Abs or modulated by recombinant forms of ligands or receptors. One or more of the costimulatory and inhibitory receptors and ligands that regulate T cell responses, other than PD-1/PD-L1, may provide targets for synergizing with the anti-MerTK Abs disclosed herein for inhibiting tumor growth. Indeed, synergistic anti-tumor efficacy has been demonstrated using a combination of the anti-MerTK Ab, 4E9, and CTLA4 blockade in an immunotherapy-resistant mouse 4T1 mammary carcinoma model as well as with anti-OX40 and anti-GITR agonist Abs in the CT26 and MC38 mouse syngeneic tumor models (data not shown).

Certain anti-MerTK-1 mAbs that are effective in enhancing the anti-tumor efficacy of checkpoint inhibitors such as anti-PD-1 and which exhibit at least one, several or all of the following desirable characteristics are provided by the present disclosure: (a) binding to hMerTK and to cMerTK with a K_{D} of about 100 nM or lower, preferably with a K_{D} of about 50 nM or lower, as determined by SPR (BIACORE®) analysis; (b) not substantially binding to human Axl or Tyro3; (c) inhibiting efferocytosis by MerTK-expressing cells with an IC₅₀ of about 1 nM or lower; (d) inhibiting the binding of Gas6 to MerTK and inhibiting hMerTK/Gas6 signaling with an IC₅₀ of about 10 nM or lower, preferably about 1 nM or lower; (e) inhibiting tumor cell growth in vivo; and (f) interacting synergistically with a checkpoint inhibitor, such as an anti-PD-1/anti-PD-L1 Ab, in reducing the growth of cancer cells in vivo. Certain anti-MerTK Abs that may be used in the therapeutic methods, compositions or kits described herein include mAbs that bind specifically to hMerTK with high affinity and exhibit at least three, and preferably all, of the preceding characteristics.

### Anti-PD-1/Anti-PD-L1 Abs Suitable for Use in the Disclosed Therapeutic Methods

Anti-PD-1 Abs suitable for use in the methods for cancer treatment, compositions or kits disclosed herein include isolated Abs, preferably mAbs or antigen-binding portions thereof, that bind to PD-1 with high specificity and affinity, block the binding of PD-L1 and/or PD-L2 to PD-1, and inhibit the immunosuppressive effect of the PD-1 signaling pathway. Similarly, anti-PD-L1 Abs suitable for use in these methods are isolated Abs, preferably mAbs or antigen-binding portions thereof, that bind to PD-L1 with high specificity and affinity, block the binding of PD-L1 to PD-1 and CD80 (B7-1), and inhibit the immunosuppressive effect of the PD-1 signaling pathway. In any of the therapeutic methods disclosed herein, an anti-PD-1 or anti-PD-L1 Ab includes an antigen-binding portion or fragment that binds to the PD-1 receptor or PD-L1 ligand, respectively, and exhibits functional properties similar to those of whole Abs in inhibiting receptor-ligand binding and reversing the inhibition of T cell activity, thereby upregulating an immune response.

### Anti-PD-1 Abs

MAbs that bind specifically to PD-1 with high affinity have been disclosed in U.S. Pat. No. 8,008,449. Other anti-PD-1 mAbs have been described in, for example, U.S. Pat. Nos. 7,488,802, 8,168,757, 8,354,509, and 9,205,148. The anti-PD-1 mAbs disclosed in U.S. Pat. No. 8,008,449 have been demonstrated to exhibit several or all of the following characteristics: (a) binding to human PD-1 with a K_{D} of about 50 nM or lower, as determined by the SPR (BIACORE®) biosensor system; (b) not substantially binding to human CD28, CTLA-4 or ICOS; (c) increasing T-cell proliferation, interferon-γ production and IL-2 secretion in a Mixed Lymphocyte Reaction (MLR) assay; (d) binding to human PD-1 and cynomolgus monkey PD-1; (e) inhibiting the binding of PD-L1 and PD-L2 to PD-1; (f) releasing inhibition imposed by Treg cells on proliferation and interferon-γ production of CD4⁺CD25⁻ T cells; (g) stimulating antigen-specific memory responses; (h) stimulating Ab responses; and (i) inhibiting tumor cell growth in vivo. Anti-PD-1 Abs usable in the disclosed methods of treatment, compositions or kits include mAbs that bind specifically to human PD-1 with high affinity and exhibit at least five, and preferably all, of the preceding characteristics. For example, an anti-PD-1 Ab suitable for use in the therapeutic methods disclosed herein (a) binds to human PD-1 with a K_{D} of about 10 nM to 0.1 nM, as determined by SPR (BIACORE®); (b) increases T-cell proliferation, interferon-γ production and IL-2 secretion in a MLR assay; (c) inhibits the binding of PD-L1 and PD-L2 to PD-1; (d) reverses inhibition imposed by Tregs on proliferation and interferon-γ production of CD4⁺CD25⁻ T cells; (e) stimulates antigen-specific memory responses; and (f) inhibits tumor cell growth in vivo.

Other anti-PD-1 mAbs have been described in, for example, U.S. Pat. Nos. 6,808,710, 7,488,802, 8,168,757 and 8,354,509, U.S. Publication No. 2016/0272708, and PCT Publication Nos. WO 2008/156712, WO 2012/145493, WO 2014/179664, WO 2014/194302, WO 2014/206107, WO 2015/035606, WO 2015/085847, WO 2015/112800, WO 2015/112900, WO 2016/106159, WO 2016/197367, WO 2017/020291, WO 2017/020858, WO 2017/024465, WO 2017/024515, WO 2017/025016, WO 2017/025051, WO 2017/040790, WO 2017/106061, WO 2017/123557, WO 2017/132827, WO 2017/133540, each of which is incorporated by reference in its entirety.

In certain embodiments, the anti-PD-1 mAb is selected from the group consisting of nivolumab (OPDIVO®; formerly designated 5C4, BMS-936558, MDX-1106, or ONO-4538), pembrolizumab (KEYTRUDA®; formerly designated lambrolizumab and MK-3475; see WO 2008/156712A1), PDR001 (see WO 2015/112900), MEDI-0680 (formerly designated AMP-514; see WO 2012/145493), REGN-2810 see WO 2015/112800), JS001 (see Liu and Wu, 2017), BGB-A317 (see WO 2015/035606 and US 2015/0079109), INCSHR1210 (SHR-1210; see WO 2015/085847; Liu and Wu, 2017), TSR-042 (ANB011; see WO 2014/179664), GLS-010 (WBP3055; see Liu and Wu, 2017), AM-0001 (see WO 2017/123557), STI-1110 (see WO 2014/194302), AGEN2034 (see WO 2017/040790), and MGD013 (see WO 2017/106061).

In certain preferred embodiments of any of the therapeutic methods described herein comprising administration of an anti-PD-1 Ab, the anti-PD-1 Ab is nivolumab, OPDIVO®), which has already been approved by the U.S. Food and Drug Administration (FDA) for treating multiple different cancers. Nivolumab is a fully human IgG4 (S228P) PD-1 immune checkpoint inhibitor Ab that selectively prevents interaction with PD-1 ligands (PD-L1 and PD-L2), thereby blocking the down-regulation of antitumor T-cell functions (described as mAb C5 in U.S. Pat. No. 8,008,449; Wang et al., 2014). In other preferred embodiments, the anti-PD-1 Ab is pembrolizumab (KEYTRUDA®; a humanized monoclonal IgG4 Ab directed against PD-1 and described as h409A11 in U.S. Pat. No. 8,354,509), which has also been approved for multiple cancer indications.

Anti-PD-1 Abs usable in the disclosed methods, compositions or kits also include isolated Abs, preferably mAbs, that bind specifically to human PD-1 (hPD-1) and cross-compete for binding to human PD-1 with any one of the anti-PD-1 Abs described herein, e.g.: nivolumab (5C4; see, e.g., U.S. Pat. No. 8,008,449; WO 2013/173223) and pembrolizumab. Abs that cross-compete with a reference Ab, e.g., nivolumab or pembrolizumab, for binding to an antigen, in this case human PD-1, can be readily identified in standard PD-1 binding assays such as BIACORE® analysis, ELISA assays or flow cytometry (see, e.g., WO 2013/173223). In certain embodiments, the anti-PD-1 Ab binds to the same epitope as any of the anti-PD-1 Abs described herein, e.g., nivolumab or pembrolizumab.

An anti-PD-1 Ab usable in the methods of the disclosed invention also includes an antigen-binding portion, including a Fab, F(ab′)₂, Fd or Fv fragment, a sdAb, a scFv, di-scFv or bi-scFv, a diabody, a minibody or an isolated CDR (see Hollinger and Hudson, 2005; Olafsen and Wu, 2010, for further details).

In certain embodiments, the isolated anti-PD-1 Ab or antigen-binding portion thereof comprises a heavy chain constant region which is of a human IgG1, IgG2, IgG3 or IgG4 isotype. In certain preferred embodiments, the anti-PD-1 Ab or antigen-binding portion thereof comprises a heavy chain constant region which is of a human IgG4 isotype. In other embodiments, the anti-PD-1 Ab or antigen-binding portion thereof is of a human IgG1 isotype. In certain other embodiments, the IgG4 heavy chain constant region of the anti-PD-1 Ab or antigen-binding portion thereof contains an S228P mutation (numbered using the Kabat system; Kabat et al., 1983) which replaces a serine residue in the hinge region with the proline residue normally found at the corresponding position in IgG1 isotype Abs. This mutation, which is present in nivolumab, prevents Fab arm exchange with endogenous IgG4 Abs, while retaining the low affinity for activating Fc receptors associated with wild-type IgG4 Abs (Wang et al., 2014). In yet other embodiments, the Ab comprises a light chain constant region which is a human kappa or lambda constant region.

In other embodiments of the present methods, the anti-PD-1 Ab or antigen-binding portion thereof is a mAb or an antigen-binding portion thereof. For administration to human subjects, the anti-PD-1 Ab is preferably a chimeric Ab or, more preferably, a humanized or human Ab. Such chimeric, humanized or human mAbs can be prepared and isolated by methods well known in the art, e.g., as described in U.S. Pat. No. 8,008,449.

Anti-PD-L1 Abs

Because anti-PD-1 and anti-PD-L1 target the same signaling pathway and have been shown in clinical trials to exhibit comparable levels of efficacy in a variety of cancers (see, e.g., Brahmer et al., 2012; WO 2013/173223), an anti-PD-L1 Ab may be substituted for the anti-PD-1 Ab in the combination therapy methods disclosed herein.

Anti-PD-L1 Abs suitable for use in the disclosed methods, compositions or kits are isolated Abs that bind to PD-L1 with high specificity and affinity, block binding of PD-L1 to PD-1 and to CD80, and inhibit the immunosuppressive effect of the PD-1 signaling pathway. MAbs that bind specifically to PD-L1 with high affinity have been disclosed in U.S. Pat. No. 7,943,743. Other anti-PD-L1 mAbs have been described in, for example, U.S. Pat. Nos. 8,217,149, 8,779,108, 9,175,082 and 9,624,298, and PCT Publication No. WO 2012/145493. The anti-PD-1 HuMAbs disclosed in U.S. Pat. No. 7,943,743 have been demonstrated to exhibit one or more of the following characteristics: (a) binding to human PD-1 with a K_{D} of about 50 mM or lower, as determined by SPR (BIACORE®); (b) increasing T-cell proliferation, interferon-γ production and IL-2 secretion in a MLR assay; (c) stimulating Ab responses; (d) inhibiting the binding of PD-L1 to PD-1; and (e) reversing the suppressive effect of Tregs on T cell effector cells and/or dendritic cells. Anti-PD-L1 Abs for use in the therapeutic methods disclosed herein include isolated Abs, preferably mAbs, that bind specifically to human PD-L1 with high affinity and exhibit at least one, in some embodiments at least three, and preferably all, of the preceding characteristics. For example, an anti-PD-L1 Ab suitable for use in these methods (a) binds to human PD-1 with a K_{D} of about 50 mM to 0.1 mM, as determined by surface plasmon resonance (BIACORE®); (b) increases T-cell proliferation, interferon-γ production and IL-2 secretion in a MLR assay; (c) inhibits the binding of PD-L1 to PD-1 and to CD80; and (d) reverses the suppressive effect of Tregs on T cell effector cells and/or dendritic cells.

A suitable anti-PD-L1 Ab for use in the present methods is BMS-936559 (formerly MDX-1105; designated 12A4 in U.S. Pat. No. 7,943,743). Other suitable anti-PD-L1 Abs include atezolizumab (TECENTRIQ®; previously known as RG7446 and MPDL3280A; designated YW243.55S70 in U.S. Pat. No. 8,217,149; see, also, Herbst et al., 2014), durvalumab (IMFINZI®; previously known as MEDI-4736; designated 2.14H9OPT in U.S. Pat. No. 8,779,108), avelumab (BAVENCIO®; previously known as MSB-0010718C; designated A09-246-2 in U.S. Pat. No. 9,624,298), STI-A1014 (designated H6 in U.S. Pat. No. 9,175,082), CX-072 (see WO 2016/149201), KNO35 (see Zhang et al., 2017), LY3300054 (see, e.g., WO 2017/034916), and CK-301 (see Gorelik et al., 2017).

Anti-PD-L1 Abs suitable for use in the disclosed methods, compositions or kits also include isolated Abs that bind specifically to human PD-L1 and cross-compete for binding to human PD-L1 with a reference Ab which may be any one of the anti-PD-L1 Abs disclosed herein, e.g., BMS-936559 (12A4; see, e.g., U.S. Pat. No. 7,943,743; WO 2013/173223), atezolizumab, durvalumab, avelumab or STI-A1014. The ability of an Ab to cross-compete with a reference Ab for binding to human PD-L1 demonstrates that such Ab binds to the same epitope region of PD-L1 as the reference Ab and is expected to have very similar functional properties to that of the reference Ab by virtue of its binding to substantially the same epitope region of PD-L1. In some embodiments, the anti-PD-L1 Ab binds the same epitope as any of the anti-PD-L1 Abs described herein, e.g., atezolizumab, durvalumab, avelumab or STI-A1014. Cross-competing Abs can be readily identified based on their ability to cross-compete with a reference Ab such as atezolizumab or avelumab in standard PD-L1 binding assays such as BIACORE® analysis, ELISA assays or flow cytometry that are well known to persons skilled in the art (see, e.g., WO 2013/173223).

In certain preferred embodiments, the isolated anti-PD-L1 Abs for use in the present methods are mAbs. In other embodiments, especially for administration to human subjects, these Abs are preferably chimeric Abs, or more preferably humanized or human Abs. Chimeric, humanized and human Abs can be prepared and isolated by methods well known in the art, e.g., as described in U.S. Pat. No. 7,943,743.

In certain embodiments, the anti-PD-L1 Ab or antigen-binding portion thereof comprises a heavy chain constant region which is of a human IgG1, IgG2, IgG3 or IgG4 isotype. In certain other embodiments, the anti-PD-L1 Ab or antigen-binding portion thereof is of a human IgG1 of IgG4 isotype. In further embodiments, the sequence of the IgG4 heavy chain constant region of the anti-PD-L1 Ab or antigen-binding portion thereof contains an S228P mutation. In other embodiments, the Ab comprises a light chain constant region which is a human kappa or lambda constant region.

Anti-PD-L1 Abs of the invention also include antigen-binding portions of the above Abs, including Fab, F(ab′)2, Fd, Fv, and scFv, di-scFv or bi-scFv, and scFv-Fc fragments, nanobodies, diabodies, triabodies, tetrabodies, and isolated CDRs, that bind to PD-L1 and exhibits functional properties similar to those of whole Abs in inhibiting receptor binding and up-regulating the immune system.

### Therapeutic Methods

Treatment of Cancer with an Anti-MerTK Ab as Monotherapy

This disclosure provides a method for treating a subject afflicted with a cancer, comprising administering to the subject a therapeutically effective amount of any one of the anti-MerTK Abs, immunoconjugates or bispecific molecules disclosed herein, or a pharmaceutical composition comprising any one of said anti-MerTK Abs, immunoconjugates or bispecific molecules, such that the subject is treated.

The disclosure also provides a method for inhibiting growth of tumor cells in a subject, comprising administering to the subject a therapeutically effective amount any one of the anti-MerTK Abs, immunoconjugates or bispecific molecules disclosed herein, or a pharmaceutical composition comprising any one of said anti-MerTK Abs, immunoconjugates or bispecific molecules, such that growth of tumor cells in the subject is inhibited.

As described in Examples 6-8, three different anti-MerTK moMAbs, 2D9, 4E9 and 16B9, showed only slight inhibition of tumor growth in the MC38 and CT26 colon adenocarcinoma tumor models, but showed very potent antitumor activity when combined with an anti-PD-1 Ab in these models (see Examples 4-8). Thus, in certain physiological contexts, anti-MerTK Abs have been shown to be much more effective in inhibiting tumor growth when combined with a checkpoint inhibitor such as an anti-PD-1 Ab compared to monotherapy with the anti-MerTK Abs.

Treatment of Cancer with an Anti-MerTK Ab in Combination with Another Anti-Cancer Agent

This disclosure provides a method for treating a subject afflicted with a cancer, comprising administering to the subject a therapeutically effective amount of: (a) any one of the anti-MerTK Abs, immunoconjugates or bispecific molecules disclosed herein, or a pharmaceutical composition comprising any one of said anti-MerTK Abs, immunoconjugates or bispecific molecules; and (b) an additional therapeutic agent for treating cancer, such that the subject is treated.

The disclosure also a method for inhibiting growth of tumor cells in a subject, comprising administering to the subject a therapeutically effective amount of: (a) any one of the anti-MerTK Abs, immunoconjugates or bispecific molecules disclosed herein, or a pharmaceutical composition comprising any one of said anti-MerTK Abs, immunoconjugates or bispecific molecules; and (b) an additional therapeutic agent for treating cancer, such that growth of tumor cells in the subject is inhibited.

In certain preferred embodiments of any of the present methods, the subject is a human patient. In other preferred embodiments, the anti-MerTK Ab inhibits efferocytosis by the MerTK-expressing macrophage. In further embodiments, the MerTK Ab binds to a Bin2 epitope of hMerTK.

In certain embodiments, the additional therapeutic agent is a compound that reduces inhibition of the immune system. For example, the additional therapeutic agent may be a small-molecule compound, a macrocyclic peptide, a fusion protein, or an Ab. In further embodiments, the additional therapeutic agent is an antagonistic Ab that binds specifically to PD-1, PD-L1, CTLA-4, LAG-3, BTLA, TIM-3, KIR, KLRG-1, A2aR, TIGIT, the VISTA receptor, CD244, or CD160. In other embodiments, the additional therapeutic agent is an agonistic Ab that binds specifically to ICOS, CD137, CD134, CD27, GITR or HVEM. The data presented in Examples 4-8 confirm the hypothesis that inhibition of MerTK-mediated efferocytosis results in increased antigen presentation, costimulation and proinflammatory cytokine production in the tumor microenvironment, thereby sensitizing tumors to T cell-directed immunotherapies. In certain preferred embodiments, the additional therapeutic agent is an antagonistic Ab or antigen-binding portion thereof that binds specifically to PD-1. In other preferred embodiments, the additional therapeutic agent is an antagonistic Ab or antigen-binding portion thereof that binds specifically to PD-L1. In further embodiments, the additional therapeutic agent is an antagonistic Ab or antigen-binding portion thereof that binds specifically to CTLA-4.

### Cancers Amenable to Treatment by Disclosed Methods

Immuno-oncology, which relies on using the practically infinite flexibility of the immune system to attack and destroy cancer cells, is applicable to treating a very broad range of cancers (see, e.g., Yao et al., 2013; Callahan et al., 2016; Pianko et al., 2017; Farkona et al., 2016; Kamta et al., 2017). The anti-PD-1 Ab, nivolumab, has been shown to be effective in treating many different types of cancers (see, e.g., Brahmer et al., 2015; Guo et al., 2017; Pianko et al., 2017; WO 2013/173223), and is currently undergoing clinical trials in multiple solid and hematological cancers. Accordingly, the disclosed methods employing blockade of the MerTK receptor or dual blockade of the PD-1 and MerTK receptors are applicable to treating a wide variety of both solid and liquid tumors.

Broad Spectrum of Cancers Amenable to Treatment

Because the Abs used in the cancer treatment methods disclosed herein do not directly target cancer cells but, instead, target and enhance the immune system by dual blockade of the PD-1 signaling pathway and MerTK-mediated efferocytosis, facilitating the enhanced immune system in attacking and destroying cancer cells, these Abs are applicable to the treatment of a broad range of cancers. The efficacy of nivolumab in treating diverse cancers has already been demonstrated, evidenced by the approval of this drug to treat advanced melanoma, advanced non-small cell lung cancer, metastatic renal cell carcinoma, classical Hodgkin lymphoma, advanced squamous cell carcinoma of the head and neck, metastatic urothelial carcinoma, MSI-H or dMMR metastatic colorectal cancer, hepatocellular carcinoma, and small cell lung cancer (Drugs.com—Opdivo Approval History: https://www.drugs.com/history/opdivo.html), with clinical trials in many other cancers ongoing. Similarly, anti-PD-L1 drugs such as atezolizumab (TECENTRIQ®), durvalumab (IMFINZI®) and avelumab (BAVENCIO®) have been gaining approvals in a variety of indications. Accordingly, a wide variety of different cancers are treatable using the anti-MerTK Ab, and especially the combination of anti-MerTK and anti-PD-1/PD-L1 Abs. The high efficacy demonstrated for this combination of therapeutics allows a focus on cancers plagued by large unmet medical need.

In certain embodiments, the disclosed combination therapy methods may be used to treat a cancer which is a solid tumor. The present combination may be particularly effective in patients with rapidly progressing disease or rapid progression on checkpoint inhibitor therapy, where immediate tumor de-bulking is needed and an immunogenic boost may prove efficacious. Thus, in certain embodiments, the solid tumor is a cancer selected from small cell lung cancer (SCLC), squamous non-small cell lung cancer (NSCLC), non-squamous NSCLC, and triple negative breast cancer (TNBC).

The combination of an anti-MerTK Ab of the invention and a checkpoint inhibitor such as an anti-PD-1/PD-L1 Ab may also be effective in earlier phases of disease where chemotherapy and/or radiation are key treatment modalities and there is a need promote sustained anti-tumor immunity. In certain embodiments, the solid tumor is a cancer selected from esophageal cancer, gastric cancer, rectal cancer, non-small cell lung cancer (NSCLC), and squamous cell carcinoma of the head and neck (SCCHN).

In certain other embodiments, the combination therapy comprising an anti-MerTK Ab is used to treat non-inflamed tumors with a high macrophage content to enhance tumor immunogenicity and promote inflammatory responses. For example, the combination may be used to treat a solid tumor selected from pancreatic ductal adenocarcinoma (PDAC), metastatic castration-resistant prostate cancer (mCRPC) and glioblastoma multiforme (GBM).

In certain other embodiments, the solid tumor is selected from melanoma, renal cancer, NSCLC, colorectal cancer, gastric cancer, bladder cancer and glioblastoma.

In certain other embodiments, the solid tumor is a cancer selected from SCLC, NSCLC, squamous NSCLC, non-squamous NSCLC, squamous cell cancer, pancreatic cancer (PAC), pancreatic ductal adenocarcinoma (PDAC), ovarian cancer, cervical cancer, carcinoma of the fallopian tubes, uterine (endometrial) cancer, carcinoma of the endometrium, uterine sarcoma, carcinoma of the cervix, carcinoma of the vagina, carcinoma of the vulva, cancer of the urethra, cancer of the ureter, prostate cancer, metastatic castration-resistant prostate cancer (mCRPC), testicular cancer, penile cancer, bladder cancer, breast cancer, triple negative breast cancer (TNBC), male breast cancer, germ cell tumor, sarcoma, skin cancer, basal cell carcinoma, squamous cell carcinoma, Merkel cell carcinoma, bone cancer, melanoma, head and neck cancer, squamous cell carcinoma of the head and neck (SCCHN), thyroid cancer, oral cancer, mouth cancer, salivary gland cancer, throat cancer, esophageal cancer, gastrointestinal cancer, gastric cancer, cancer of the small intestine, gallbladder and bile duct cancer, colorectal cancer, colon carcinoma, rectal cancer, anal cancer, liver cancer, hepatoma, kidney cancer, renal cell carcinoma, cancer of the endocrine system, tumors of the thymus gland, thymona, cancer of the parathyroid gland, cancer of the adrenal gland, soft tissue sarcoma, mesothelioma, carcinoma of the renal pelvis, neoplasm of the central nervous system (CNS), primary CNS lymphoma, tumor angiogenesis, spinal axis tumor, brain cancer, glioma, brain stem glioma, glioblastoma, glioblastoma multiforme (GBM), neuroblastoma, pituitary adenoma, epidermoid cancer, solid tumors of childhood, pediatric sarcoma, rhabdomyosarcoma, metastatic cancer, cancer of unknown primary origin, environmentally-induced cancers, virus-related cancers, AIDS-related cancers, Kaposi's sarcoma, cancers of viral origin, advanced, refractory and/or recurrent solid tumors, and any combination of the preceding solid tumors. In certain embodiments, the cancer is an advanced, unresectable, metastatic, refractory cancer, and/or recurrent cancer.

In certain embodiments, the present combination therapy methods may be used to treat a cancer which is a hematological malignancy. Hematological malignancies include liquid tumors derived from either of the two major blood cell lineages, i.e., the myeloid cell line (which produces granulocytes, erythrocytes, thrombocytes, macrophages and mast cells) or the lymphoid cell line (which produces B, T, NK and plasma cells), including all types of leukemias, lymphomas, and myelomas. Hematological malignancies that may be treated using the present combination therapy methods include, for example, cancers selected from acute lymphoblastic leukemia (ALL), acute myelogenous leukemia (AML), chronic lymphocytic leukemia (CLL), chronic myelogenous leukemia (CML), Hodgkin's lymphoma (HL), non-Hodgkin's lymphomas (NHLs), multiple myeloma, smoldering myeloma, monoclonal gammopathy of undetermined significance (MGUS), advanced, metastatic, refractory and/or recurrent hematological malignancies, and any combinations of said hematological malignancies.

In other embodiments, the hematological malignancy is a cancer selected from acute, chronic, lymphocytic (lymphoblastic) and/or myelogenous leukemias, such as ALL, AML, CLL, and CML; lymphomas, such as HL, NHLs, of which about 85% are B cell lymphomas, including diffuse large B-cell lymphoma (DLBCL), follicular lymphoma (FL), chronic lymphocytic leukemia (CLL)/small lymphocytic lymphoma (SLL), mantle cell lymphoma, marginal zone B-cell lymphomas (mucosa-associated lymphoid tissue (MALT) lymphoma, nodal marginal zone B-cell lymphoma, and splenic marginal zone B-cell lymphoma), Burkitt lymphoma, lymphoplasmacytoid lymphoma (LPL; also known as Waldenstrom's macroglobulinemia (WM)), hairy cell lymphoma, and primary central nervous system (CNS) lymphoma, NHLs that are T cell lymphomas, including precursor T-lymphoblastic lymphoma/leukemia, T-lymphoblastic lymphoma/leukemia (T-Lbly/T-ALL), peripheral T-cell lymphomas such as cutaneous T-cell lymphoma (CTLC, i.e., mycosis fungoides, Sezary syndrome and others), adult T-cell lymphoma/leukemia, angioimmunoblastic T-cell lymphoma, extranodal natural killer/T-cell lymphoma nasal type, enteropathy-associated intestinal T-cell lymphoma (EATL), anaplastic large-cell lymphoma (ALCL), and peripheral T-cell lymphoma unspecified, acute myeloid lymphoma, lymphoplasmacytoid lymphoma, monocytoid B cell lymphoma, angiocentric lymphoma, intestinal T-cell lymphoma, primary mediastinal B-cell lymphoma, post-transplantation lymphoproliferative disorder, true histiocytic lymphoma, primary effusion lymphoma, diffuse histiocytic lymphoma (DHL), immunoblastic large cell lymphoma, and precursor B-lymphoblastic lymphoma; myelomas, such as multiple myeloma, smoldering myeloma (also called indolent myeloma), monoclonal gammopathy of undetermined significance (MGUS), solitary plasmocytoma, IgG myeloma, light chain myeloma, nonsecretory myeloma, and amyloidosis; and any combinations of said hematological malignancies. The present methods are also applicable to treatment of advanced, metastatic, refractory and/or recurrent hematological malignancies.

### Medical Uses of Anti-MerTK and Anti-PD-1/Anti-PD-L1 Abs

This disclosure also provides an isolated anti-MerTK Ab, preferably a mAb or an antigen-binding portion thereof, for use in a method for treating a subject afflicted with a cancer. The disclosure further provides an isolated anti-MerTK Ab, preferably a mAb or an antigen-binding portion thereof, and a checkpoint inhibitor such as an isolated anti-PD-1/anti-PD-L1 Ab, preferably a mAb or an antigen-binding portion thereof, for use in combination in a method for treating a subject afflicted with cancer comprising dual blockade of efferocytosis and of the checkpoint pathway, e.g., the PD-1/PD-L1 signaling pathway. The anti-MerTK Ab may be used as monotherapy or in combination with a checkpoint inhibitor, such as anti-PD-1/anti-PD-L1 Ab, for treatment of the full range of cancers disclosed herein.

One aspect of the disclosed invention entails the use of an isolated anti-MerTK Ab or an antigen-binding portion thereof of the invention for the preparation of a medicament for treating a subject afflicted with a cancer. The anti-MerTK Ab may be used alone or in combination with a checkpoint inhibitor such as an isolated anti-PD-1/anti-PD-L1 Ab or an antigen-binding portion thereof for the preparation of the medicament for treating the cancer patient. Uses of any such anti-MerTK Ab and anti-PD-1/anti-PD-L1 Ab for the preparation of medicaments are broadly applicable to the full range of cancers disclosed herein.

This disclosure also provides an anti-MerTK Ab or an antigen-binding portion thereof in combination with a checkpoint inhibitor such as an isolated anti-PD-1/anti-PD-L1 Ab or an antigen-binding portion thereof for use in methods of treating cancer corresponding to all the embodiments of the methods of treatment employing this combination of therapeutics described herein.

### Pharmaceutical Compositions and Dosage Regimens

Abs used in the any of the therapeutic methods disclosed herein may be constituted in a composition, e.g., a pharmaceutical composition containing an Ab and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. Preferably, the carrier for a composition containing an Ab is suitable for intravenous (IV), intramuscular, subcutaneous (SC), parenteral, spinal or epidermal administration (e.g., by injection or infusion).

An option for SC injection is based on Halozyme Therapeutics' ENHANZE® drug-delivery technology, involving a co-formulation of an Ab with recombinant human hyaluronidase enzyme (rHuPH20) that removes traditional limitations on the volume of biologics and drugs that can be delivered subcutaneously due to the extracellular matrix (U.S. Pat. No. 7,767,429). It may be possible to co-formulate two Abs used in combination therapy into a single composition for SC administration.

A pharmaceutical composition of the invention may include one or more pharmaceutically acceptable salts, anti-oxidants, aqueous and non-aqueous carriers, and/or adjuvants such as preservatives, wetting agents, emulsifying agents and dispersing agents.

Dosage regimens are adjusted to provide the optimum desired response, e.g., a maximal therapeutic response and/or minimal adverse effects. For administration of an anti-MerTK, anti-PD-1 or anti-PD-L1 Ab or an antigen-binding portion thereof, including for combination use, the dosage may range from about 0.01 to about 20 mg/kg, preferably from about 0.1 to about 10 mg/kg, of the subject's body weight. For example, dosages can be about 0.1, 0.3, 1, 2, 3, 5 or 10 mg/kg body weight, and more preferably, about 0.3, 1, 3, or 10 mg/kg body weight. Alternatively, a fixed or flat dose, e.g., about 50-2000 mg of the Ab or antigen-binding portion thereof, instead of a dose based on body weight, may be administered. The dosing schedule is typically designed to achieve exposures that result in sustained receptor occupancy (RO) based on typical pharmacokinetic properties of an Ab. An exemplary treatment regime entails administration once per week, once every 2 weeks, once every 3 weeks, once every 4 weeks, once a month, once every 3-6 months or longer. In certain preferred embodiments, the anti-MerTK, anti-PD-1 or anti-PD-L1 Ab or antigen-binding portion thereof is administered to the subject once every 2 weeks. In other preferred embodiments, the Ab or antigen-binding portion thereof is administered once every 3 weeks. The dosage and scheduling may change during a course of treatment.

When used in combinations, a subtherapeutic dosage of one or both Abs, e.g., a dosage of an anti-MerTK, anti-PD-1 and/or anti-PD-L1 Ab or antigen-binding portion thereof lower than the typical or approved monotherapy dose, may be used. For example, a dosage of nivolumab that is lower than the approved 3 mg/kg every 2 weeks, for instance, 1.0 mg/kg or less every 2, 3 or 4 weeks, is regarded as a subtherapeutic dosage. RO data from 15 subjects who received 0.3 mg/kg to 10 mg/kg dosing with nivolumab indicate that PD-1 occupancy appears to be dose-independent in this dose range. Across all doses, the mean occupancy rate was 85% (range, 70% to 97%), with a mean plateau occupancy of 72% (range, 59% to 81%) (Brahmer et al., 2010). Thus, 0.3 mg/kg dosing may allow for sufficient exposure to lead to significant biologic activity.

The synergistic interaction observed in mouse tumor models between the anti-MerTK and anti-PD-1/anti-PD-L1 Abs or antigen-binding portions thereof may permit the administration of one or both of these therapeutics to a cancer patient at subtherapeutic dosages. In certain embodiments of the disclosed combination therapy methods, the anti-MerTK Ab or antigen-binding portion thereof is administered at a subtherapeutic dose to a cancer patient. In other embodiments, the anti-PD-1/anti-PD-L1 Ab or antigen-binding portion thereof is administered to the patient at a subtherapeutic dose. In further embodiments, the anti-PD-1/anti-PD-L1 and anti-MerTK Abs or antigen-binding portions thereof are each administered to the patient at a subtherapeutic dose.

The administration of such a subtherapeutic dose of one or both Abs may reduce adverse events compared to the use of higher doses of the individual Abs in monotherapy. Thus, the success of the disclosed methods of combination therapy may be measured not only in improved efficacy of the combination of Abs relative to monotherapy with these Abs, but also in increased safety, i.e., a reduced incidence of adverse events, from the use of lower dosages of the drugs in combination relative to the monotherapy doses.

In certain embodiments of any of the methods disclosed herein, the anti-MerTK, anti-PD-1 and/or anti-PD-L1 Abs are formulated for intravenous (IV) administration or for subcutaneous (SC) injection. In certain embodiments, the anti-MerTK Ab or antigen-binding portion thereof and the anti-PD-1/anti-PD-L1 Ab or antigen-binding portion thereof are administered sequentially to the subject. “Sequential” administration means that one of the anti-MerTK and anti-PD-1/anti-PD-L1 Abs is administered before the other. Either Ab may be administered first; i.e., in certain embodiments, the anti-PD-1/anti-PD-L1 Ab is administered before the anti-MerTK Ab, whereas in other embodiments, the anti-MerTK Ab is administered before the anti-PD-1/anti-PD-L1 Ab. In certain embodiments, each Ab is administered by IV infusion, for example, by infusion over a period of about 60 minutes. In other embodiments, at least one Ab is administered by SC injection.

In certain embodiments of sequential IV administration, for the convenience of the patient, the anti-MerTK and anti-PD-1/anti-PD-L1 Abs or portions thereof are administered within 30 minutes of each other. Typically, when both the anti-MerTK and anti-PD-1/anti-PD-L1 Abs are to be delivered by IV administration on the same day, separate infusion bags and filters are used for each infusion. The infusion of the first Ab is promptly followed by a saline flush to clear the line of the Ab before starting the infusion of the second Ab. In other embodiments, the two Abs are administered within 1, 2, 4, 8, 24 or 48 h of each other.

The delivery of at least one Ab by SC administration reduces health care practitioner time required for administration and shortens the time for drug administration. For example, the use of SC injection could cut the time needed for IV administration, typically about 30-60 min, to about 5 min. In certain embodiments of sequential SC administration, the anti-MerTK and anti-PD-1/anti-PD-L1 Abs or portions thereof are administered within 10 min of each other.

Because checkpoint inhibitor Abs have been shown to produce very durable responses, in part due to the memory component of the immune system (see, e.g., WO 2013/173223; Lipson et al., 2013; Wolchok et al., 2013), the activity of an administered anti-PD-1/anti-PD-L1 Ab may be ongoing for several weeks, several months, or even several years. In certain embodiments, the present combination therapy methods involving sequential administration entail administration of an anti-MerTK Ab to a patient who has been previously treated with an anti-PD-1/anti-PD-L1 Ab. In further embodiments, the anti-MerTK Ab is administered to a patient who has been previously treated with, and progressed on, an anti-PD-1/anti-PD-L1 Ab. In other embodiments, the present combination therapy methods involving sequential administration entail administration of an anti-PD-1/anti-PD-L1 Ab to a patient who has been previously treated with an anti-MerTK Ab, optionally a patient whose cancer has progressed after treatment with the anti-MerTK Ab.

In certain other embodiments, the anti-PD-1/anti-PD-L1 and anti-MerTK Abs are administered concurrently, either admixed as a single composition in a pharmaceutically acceptable formulation for concurrent administration, or concurrently as separate compositions with each Ab in formulated in a pharmaceutically acceptable composition.

### Kits

Also within the scope of the present invention are kits comprising an anti-MerTK Ab and an anti-PD-1/anti-PD-L1 Ab for therapeutic uses. Kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a cancer, the kit comprising: (a) one or more dosages ranging from about 0.1 to about 20 mg/kg body weight of a mAb or an antigen-binding portion thereof that binds specifically to MerTK; and (b) instructions for using the mAb or portion thereof in any of the therapeutic methods disclosed herein. The disclosure further provides a kit for treating a subject afflicted with a cancer, the kit comprising: (a) one or more dosages ranging from about 0.1 to about 20 mg/kg body weight of a mAb or an antigen-binding portion thereof that binds specifically to MerTK; (b) one or more dosages of a checkpoint inhibitor such as about 3 mg/kg body weight or 200 to about 1600 mg of an anti-PD-1/anti-PD-L1 mAb or an antigen-binding portion thereof; and (c) instructions for using the anti-MerTK mAb and the checkpoint inhibitor, e.g., the anti-PD-1/anti-PD-L1 mAb, in any of the combination therapy methods disclosed herein.

In certain embodiments, the Abs may be co-packaged in unit dosage form. In certain preferred embodiments for treating human patients, the kit comprises an anti-human PD-1 Ab disclosed herein, e.g., nivolumab or pembrolizumab.

The present invention is further illustrated by the following examples which should not be construed as further limiting. The contents of all references cited throughout this application are expressly incorporated herein by reference.

### Example 1

### Generation of MAbs Against MERTK

Human and mouse anti-MerTK mAbs were generated by immunizing transgenic mice that express human Ab genes with a human MerTK (hMerTK) antigen to raise in the mice a repertoire of human Ig's specific for MerTK, and by immunizing MerTK knock-out mice with a mouse MerTK (mMerTK) antigen or a mixture of mMerTK and hMerTK antigens.

### Immunization of Human Immunoglobulin Transgenic Mice

HuMAbs to hMerTK were generated by immunizing human Ig transgenic mice, strain Hco42:01 [J/K] (HCo42(289729p)+\hat{ };JHD++;JKD++;KCo5(9272)+\hat{ };) (Lonberg, 1994; Lonberg et al., 1994), with recombinant hMerTK-mFc fusion protein (R&D Systems, Minneapolis, Minn.) comprising the extracellular portion of hMerTK linked to the mouse IgG2a Fc at its C-terminus. The antigen was mixed 1:1 with Ribi adjuvant and mice were immunized at weekly intervals intraperitoneally and subcutaneously. Serum titers were monitored after four and six injections. Mice received two final boosts of hMerTK-mFc protein by intravenous (IV) and intraperitoneal (IP) injection 2 and 3 days prior to the final harvest. Both lymph nodes and spleen were harvested for subsequent fusions.

### Immunization of MerTK Knock-Out Mice

Mouse anti-MerTK mAbs were generated by immunizing MerTK knock-out (KO) mice with recombinant mMerTK-hFc fusion protein (R&D Systems) mixed with hMerTK-hFc fusion protein (R&D Systems) or with miMerTK-hFc alone. The antigens were mixed 1:1 with Ribi adjuvant and injected at weekly intervals using footpad immunizations. Serum titers were monitored after 4 injections and then mice received 2 final footpad boosts 2 and 3 days prior to the final harvest. Lymph nodes were harvested for subsequent fusions.

### Generation of Hybridomas Producing MAbs to MerTK

Mouse lymphocytes were isolated from immunized mice as described above, and hybridomas were generated by fusions with a mouse myeloma fusion partner by electric field based electrofusion using a Cyto Pulse Hybrimmune large chamber cell fusion electroporator (BTX/Harvard Apparatus, Holliston, Mass.). Single cell suspensions of lymphocytes from immunized mice were fused to an equal number of P3X63 Ag8.6.53 (ATCC) non-secreting mouse myeloma cells (fusion numbers 5760-5763 for human Ig transgenic mice and 5712 and 5775 for MerTK KO mice). The resulting cells were plated in flat-bottom microliter plates in Medium E (StemCell Technologies, Seattle, Wash.) supplemented with aminopterin (Sigma-Aldrich, St. Louis, Mo.) for selection of hybridomas.

### Example 2

### Screening and Selection of Human Anti-Human MERTK MAbs

Screening for MAbs that Selectively Bind to Human and Cynomolgus MerTK

In order to generate HuMAbs that bind to hMerTK, human Ig transgenic mice were immunized with a hMerTK antigen as described in Example 1.

For hybridomas derived from these human Ig transgenic animals, individual wells were screened after 10 to 12 days for the presence of human IgG/human kappa light chain Abs using a homogeneous time resolved fluorescence (HIRE) assay (Cisbio, Bedford, Mass.). Hybridoma supernatants from wells positive for hIgG/hκ were tested by Fluorescence Activated Cell Sorting (FACS) for binding to Chinese Hamster Ovary (CHO) cells transfected with a kinase-mutant version of full-length hMerTK. Briefly, CHO cells transfected with hMerTK were washed with cold FACS buffer (1% fetal bovine serum (FBS) in phosphate buffered saline (PBS)) and ˜1×10⁵ cells in 50 μl were aliquoted to each well of a 96-well U-bottom plate, followed by adding 50 μl of hybridoma supernatant. Samples were incubated with the cells for 30 min on ice. Cells were washed 2 times with FACS buffer. PE-conjugated goat anti-human IgG Fc specific Ab (Jackson ImmunoResearch, West Grove, Pa.) at a 1:200 dilution was added at 100 μl per sample and incubated for 30 min on ice. Cells were washed twice and transferred and read on the FACSCalibur cytometer (BD Biosciences, San Jose, Calif.). Human MerTK-positive hybridomas were also screened for cross-reactivity to cynomolgus monkey MerTK using CHO cells transfected with cynomolgus monkey MerTK by FACS using the staining protocol described above. Hybridomas were further counter-screened by FACS for selectivity, evidenced by the absence of binding to Axl and/or Tyro3 and non-specific proteins such as keyhole limpet hemocyanin (KLH). Approximately 3,300 HuMAb clones were screened and about 300 were found to be selective for MerTK and to bind to both human and cynomolgus monkey MerTK.

Functional Screening for Antagonistic Anti MerTK mAbs

The selected HuMAb clones were functionally screened using a cell based assay (Zizzo et al., 2012) was used to identify Abs that inhibited efferocytosis. A signaling assay was also used to measure target engagement and potency in inhibiting ligand (Gash)-induced signaling (Tsou et al., 2014), and the clones were counter-screened for agonist potential. A. Clones were selected for further characterization on the bases of: binding to MerTK on human cells (tumor cell lines and primary cells) with sub-nanomolar EC₅₀; binding to MerTK on cynomolgus monkey cells (transfected cell lines and primary cells) with low to sub-nM EC₅₀; inhibiting efferocytosis to more than 80% of the maximal signal with sub-nanomolar IC₅₀; and inhibiting Gash-mediated signaling by more than 80% of control with sub-nanomolar IC₅₀ and no agonistic capacity. The variable region DNA in these Abs was sequenced by next generation sequencing and about 35 HuMAbs were selected for diversity based on sequence homology and limited potential sequence liabilities, e.g., asparagine deamidation, methionine oxidation and glycosylation sites. Based on the nucleotide sequences encoding the variable regions, six sequence families were identified in the selected HuMAbs. The selected 35 HuMAbs were also analyzed using in silico methods for their immunogenicity potential based on sequence, and were tested for their potential to induce receptor internalization using standard high content methods. Any clones exhibiting potential for immunogenicity or for inducing receptor internalization were deprioritized.

### Characterization of Binding Affinity and Binding Kinetics of Anti-hMerTK HuMAbs

The affinities and binding kinetics of the selected HuMAbs were characterized by surface plasmon resonance (SPR) analysis at 37° C. with a BIACORE® instrument (GE Healthcare, Chicago, Ill.) using a CM4 sensor chip (GE Healthcare) with immobilized anti-human Fc capture reagent (GE Healthcare) and a running buffer composed of 10 mM HEPES pH 7.4, 150 mM NaCl, 0.05% (v/v) surfactant P20, and 1 g/l BSA. MerTK Abs were captured on the chip. Recombinant soluble forms of the extracellular domains of human, cynomolgus monkey, and mouse MerTK polypeptide were injected as analytes at multiple concentrations each. The resulting sensorgrams were double-referenced and fitted to a 1:1 Langmuir binding model with mass transport.

### Epitope Binning of Anti-hMerTK HuMAbs

Of the HuMAbs that showed potent antagonistic functional effects, 13 representative HuMAbs comprising the 6 sequence families were subjected to SPR binding competition studies to identify mAbs that compete for the same or an overlapping epitope on the hMerTK antigen and could, therefore, be assigned to the same epitope bin. Three epitope bins were identified, with the vast majority assigned to Bin 1: 11 mAbs were assigned to Bin 1, 1 mAb was assigned to Bin 2, and 1 mAb was assigned to Bin 3.

### Epitope Mapping by Yeast Display and Hydrogen Deuterium Exchange (HDX)

Select Abs were chosen based on the epitope binning data for epitope mapping analysis by yeast display and/or hydrogen-deuterium exchange mass spectrometry (HDX-MS) to further elucidate the Ab binding regions. Fab fragments of the mAbs were prepared and used for the HDX-MS epitope mapping as the Fab fragments gave cleaner results than the whole Abs. Bin 1 Abs were found to bind to the first Ig domain of hMerTK within a linear region spanning approximately amino acids 105 to 165 depending on the specific clone. For example, the epitope for the 8N42 Fab fragment was mapped to a region spanning amino acids 126 to 155 (¹²⁶TTISWWKDGKELLGAHHAITQFYPDDEVTA¹⁵⁵) of human MerTK (SEQ ID NO:259).

Bin 2 Abs HuMabs and moMAbs) were found to bind to the second Ig domain of MerTK within a region spanning approximately amino acids 195 to 270 depending on the specific clone. For example, the epitope for the Fab fragment of HuMAb 25B10 (from which mAbs 25J60 and 25J80 were derived) was mapped to a linear region spanning amino acids 231 to 249 (²³¹WVQNSSRVNEQPEKSPSVL²⁴⁹) of hMerTK (SEQ ID NO:259). These data were consistent with the epitope mapped by yeast display which, for mAb 25B10, identified amino acid residues N234, 5236, R237, E240, Q241, P242 and G269 as constituting the epitope. Both Bin 1 and Bin 2 binding regions are consistent with ligand blockade based on homology modeling of the Gas6/Axl crystal structure.

The single Bin 3 HuMAb binds to the Fn domains within a region spanning amino acids 420 to 490.

### Optimization of Anti-hMerTK HuMAbs

Based on their potency and duration of inhibiting efferocytosis, binding kinetics, binning diversity and sequence family diversity, certain mAbs were selected for PROmAb optimization to mitigate sequence liabilities, optimize binding affinities and revert to germline amino acids. Select mAbs were also analyzed for their biophysical properties through a variety of means such as analytical size exclusion chromatography, capillary isoelectric focusing, hydrophobicity assessments, thermal stability, and aggregation potential, to identify clones amenable for development. A mAb that was the sole selected representative of one of the sequence families was lost during PROmAb optimization; thus, 5 sequence families and 3 bins are represented in the 13 Abs that emerged from the optimization process.

Binning data and the results of the efferocytosis and signaling assays for a representative sample of 7 of the 13 selected HuMAbs (HuMAbs 1B4, 10K11, 22116, 25J60, 25J80, 8N42 and 4K10) are shown in Table 1. One HuMAb assigned to Bin 3 and two closely related Abs derived from the single HuMAb assigned to Bin 2, are included in the table, with the remaining four HuMAbs assigned to Bin 1. All 5 sequence families are represented in Table 1.

The binding kinetics data obtained for the 7 representative HuMAbs in Table 1, i.e., the dissociation constant (K_{D}), the rate constant of the binding reaction (kₒₙ), the rate constant of the dissociation reaction (k_{off}) values, and the half-life (t_{1/2}) are shown in Table 2.

The amino acid sequences for the 6 CDR domains as defined using the Kabat, Chothia and IMGT methods for HuMAbs 1B4, 10K11, 22116, 25J60, 25J80, 8N42 and 4K10 are shown in Tables 3-9, respectively.

The amino acid sequences for the V_{H}, V_{L}, heavy chain and light chain for HuMAbs 1B4, 10K11, 22116, 25J60, 25J80, 8N42 and 4K10 are shown in Tables 15-21, respectively.

TABLE 1
Binning and Functional Characterization data for Representative Anti-MerTK Abs

| | | | | Signaling Assay | | |
|---|---|---|---|---|---|---|
| | | | Functional | | hMerTK | |
| | | | Cell Assay | hMerTK | without | mMerTK |
| | | hMerTK | Inhibition of | with Gas6 | Gas6 | with Gas6 |
| | Type of | Blocking | Efferocytosis | pSTAT1 | pSTAT1 | pSTAT1 |
| mAb | mAb | Bin | IC₅₀ (nM) | IC₅₀ (nM) | IC₅₀ (nM) | IC₅₀ (nM) |
| 1B4 | human | 1 | 0.072 | 0.330 | >100 | >100 |
| 10K11 | human | 1 | 0.169 | 1.13 | >100 | nd |
| 22I16 | human | 1 | 0.352 | 3.41 | >100 | nd |
| 25J60 | human | 2 | 0.051 | 0.093 | >100 | nd |
| 25J80 | human | 2 | 0.093 | 0.227 | >100 | nd |
| 8N42 | human | 1 | 0.047 | 0.572 | >100 | nd |
| 4K10 | human | 3 | >10 | 5.42 | nd | nd |
| 2L105 | humanized | 2 | pending | 1.37 | nd | nd |
| 4M60 | humanized | 2 | pending | 8.88 | nd | nd |
| 2D9 | mouse | 2 | 0.528 | 1.44 | >100 | 2.07 |
| 4E9 | mouse | 2 | 0.632 | 1.52 | >100 | 1.86 |
| 16B9 | mouse | dnb | dnb | >100 | >100 | 0.9 |

dnb: does not bind to hMerTK-expressing cells
nd: no data

### Example 3

### Screening and Selection of Mouse Anti-MERTK MAbs

Screening for MAbs that Selectively Bind to Human and Cynomolgus MerTK

MerTK KO mice were immunized with mMerTK and hMerTK antigens to generate mouse Abs that bind to mMerTK and/or hMerTK, as described in Example 1. Supernatants from hybridomas derived from these MerTK KO mice were tested directly for binding to mouse and human MerTK CHO transfectants using fluorometric microvolume assay technology (FMAT). Hybridomas were screened by FMAT using a goat anti-mouse IgG (Fc) (Jackson ImmunoResearch) conjugated with AlexaFluor647 as a secondary reagent. Briefly, CHO cells transfected with hMERTK or mMERTK were washed and resuspended in FMAT buffer at a final concentration of 2×10⁵ cells/ml. A mixture of 1:15-diluted hybridoma supernatant and goat anti-mouse IgG FcAb used at a final concentration of 250 ng/ml was added to the cells and incubated for 2 h at room temperature. Plates were then read on the FMAT 8200 cellular detection system instrument (Applied Biosystems, Foster City, Calif.) and data analyzed using Tibco Spotfire software (Palo Alto, Calif.). Positive clones identified by FMAT were confirmed by FACS as described in Example using a PE-conjugated goat anti-mouse IgG Fc specific Ab (Jackson ImmunoResearch). Hybridomas were counter-screened by FACS to exclude clones that bound to Axl and/or Tyro3 and non-specific proteins such as KUL About 2,000 moMAb clones that bound selectively to human and/or mouse MerTK were obtained.

TABLE 2
Binding Kinetics Data for Anti-MerTK Abs

| | Human MerTK Binding Kinetics | | | | Cynomolgus MerTK Binding Kinetics | | | | Mouse MerTK Binding Kinetics | | | |
|---|---|---|---|---|---|---|---|---|---|---|---|---|
| | | | Off rate | | | | Off rate | | | | Off rate | |
| | | On rate | k_{off} × | | | On rate | k_{off} × | | | On rate | k_{off} × | |
| | K_{D} | kₐ × 10⁵ | 10⁻⁴ | t_{1/2} | K_{D} | kₐ × 10⁵ | 10⁻⁴ | t_{1/2} | K_{D} | kₐ × 10⁵ | 10⁻⁴ | t_{1/2} |
| mAb | (nM) | (1/Ms) | (1/s) | (min) | (nM) | (1/Ms) | (1/s) | (min) | (nM) | (1/Ms) | (1/s) | (min) |
| 1B4 | 34.4 | 2.5 | 84.4 | 1.4 | 54.9 | 1.45 | 79.6 | 1.5 | dnb | dnb | dnb | dnb |
| 10K11 | 9 | 1.32 | 11.7 | 9.9 | 22.5 | 1.08 | 23.5 | 4.9 | dnb | dnb | dnb | dnb |
| 22I16 | 10.6 | 0.735 | 7.75 | 14.9 | 24.7 | 0.509 | 12.6 | 10.2 | dnb | dnb | dnb | dnb |
| 25J60 | 2.3 | 4.21 | 9.75 | 11.9 | 2.5 | 4.00 | 9.9 | 11.7 | dnb | dnb | dnb | dnb |
| 25J80 | 4.1 | 2.97 | 12.3 | 9.6 | 4.5 | 2.74 | 12.4 | 4.5 | dnb | dnb | dnb | dnb |
| 8N42 | 5.7 | 2.99 | 16.8 | 6.9 | 14 | 1.00 | 14.0 | 8.3 | dnb | dnb | dnb | dnb |
| 4K10 | 27.8 | 1.21 | 33.6 | 3.4 | 31.3 | 1.21 | 37.9 | 3 | dnb | dnb | dnb | dnb |
| 2L105 | 24.8 | 0.589 | 14.6 | 7.9 | 27 | 0.559 | 15.1 | 7.7 | 13.7 | 0.895 | 12.3 | 9.4 |
| 4M60 | 50.8 | 0.264 | 13.4 | 8.6 | 42.5 | 0.322 | 13.7 | 8.4 | 47.1 | 0.529 | 24.9 | 4.6 |
| 2D9 | 23 | 0.731 | 6.4 | 18.2 | 12 | 0.533 | 6.4 | 18 | 3.2 | 1.26 | 4.03 | 28.7 |
| 4E9 | 65.1 | 0.394 | 11.4 | 10.1 | 40 | 0.284 | 11.2 | 10.3 | 9.7 | 1.37 | 13.3 | 8.7 |
| 16B9 | >250 | — | — | — | >100 | — | — | — | 35.1 | 0.727 | 25.6 | 4.5 |

dnb: does not bind to mMerTK-expressing cells and not tested

TABLE 3
Amino Acid Sequences for the 6 CDR Domains in HuMAb 1B4 as Defined using the
Kabat, Chothia and IMGT Methods

| 1B4 CDR | | | | | | |
|---|---|---|---|---|---|---|
| Defini- | Amino Acid Sequences and SEQ ID Nos. | | | | | |
| tions | VH CDR1 | VH CDR2 | VH CDR3 | VL CDR1 | VL CDR2 | VL CDR3 |
| Kabat | SGNYWG | SVDHSGSTYYSPSLKS | NTMIRGVMDWFDP | RASQGISSALA | DASSLES | QQFRSYPT |
| | (SEQ ID | (SEQ ID NO: 4) | (SEQ ID NO: 7) | (SEQ ID NO: 10) | (SEQ ID | (SEQ ID |
| | NO: 1) | | | | NO: 13) | NO: 16) |
| Chothia | GYSISSGN | DHSGS | NTMIRGVMDWFDP | RASQGISSALA | DASSLES | QQFRSYPT |
| | (SEQ ID | (SEQ ID NO: 5) | (SEQ ID NO: 8) | (SEQ ID NO: 11) | (SEQ ID | (SEQ ID |
| | NO: 2) | | | | NO: 14) | NO: 17) |
| IMGT | GYSISSGNY | VDHSGST | ARNTMIRGVMDWFDP | QGISSA | DAS | QQFRSYPT |
| | (SEQ ID | (SEQ ID NO: 6) | (SEQ ID NO: 9) | (SEQ ID NO: 12) | (SEQ ID | (SEQ ID |
| | NO: 3) | | | | NO: 15) | NO: 18) |

TABLE 4
Amino Acid Sequences for the 6 CDR Domains in HuMAb 10K11 as Defined using the
Kabat, Chothia and IMGT Methods

| 10K11 | | | | | | |
|---|---|---|---|---|---|---|
| CDR | | | | | | |
| Defini- | Amino Acid Sequences and SEQ ID Nos. | | | | | |
| tions | VH CDR1 | VH CDR2 | VH CDR3 | VL CDR1 | VL CDR2 | VL CDR3 |
| Kabat | SDYSWG | SIYHSGNTYFNPSLKS | DKSYYFGPGSMDV | RASQGISSALA | DASSLES | QQFKSYLT |
| | (SEQ ID | (SEQ ID NO: 22) | (SEQ ID NO: 25) | (SEQ ID NO: 28) | (SEQ ID | (SEQ ID |
| | NO: 19) | | | | NO: 31) | NO: 34) |
| Chothia | GYSISSDY | YHSGN | DKSYYFGPGSMDV | RASQGISSALA | DASSLES | QQFKSYLT |
| | (SEQ ID | (SEQ ID NO: 23) | (SEQ ID NO: 26) | (SEQ ID NO: 29) | (SEQ ID | (SEQ ID |
| | NO: 20) | | | | NO: 32) | NO: 35) |
| IMGT | GYSISSDYS | IYHSGNT | ARDKSYYFGPGSMDV | QGISSA | DAS | QQFKSYLT |
| | (SEQ ID | (SEQ ID NO: 24) | (SEQ ID NO: 27) | (SEQ ID NO: 30) | (SEQ ID | (SEQ ID |
| | NO: 21) | | | | NO: 33) | NO: 36) |

TABLE 5
Amino Acid Sequences for the 6 CDR Domains in HuMAb 22i16 as Defined using the
Kabat, Chothia and IMGT Methods

| 22I16 | | | | | | |
|---|---|---|---|---|---|---|
| CDR | | | | | | |
| Defini- | Amino Acid Sequences and SEQ ID Nos. | | | | | |
| tions | VH CDR1 | VH CDR2 | VH CDR3 | VL CDR1 | VL CDR2 | VL CDR3 |
| Kabat | SYSMN | YIISGSDTIFYADSVKG | DETVVRGVINYFDY | RSSQGISSALA | DASSLES | QQFISYPT |
| | (SEQ ID | (SEQ ID NO: 40) | (SEQ ID NO: 43) | (SEQ ID NO: 46) | (SEQ ID | (SEQ ID |
| | NO: 37) | | | | NO: 49) | NO: 52) |
| Chothia | GFTFSSY | ISGSDT | DETVVRGVINYFDY | RSSQGISSALA | DASSLES | QQFISYPT |
| | (SEQ ID | (SEQ ID NO: 41) | (SEQ ID NO: 44) | (SEQ ID NO: 47) | (SEQ ID | (SEQ ID |
| | NO: 38) | | | | NO: 50) | NO: 53) |
| IMGT | GFTFSSYS | IISGSDTI | ARDETVVRGVINYFDY | QGISSA | DAS | QQFISYPT |
| | (SEQ ID | (SEQ ID NO: 42) | (SEQ ID NO: 45) | (SEQ ID NO: 48) | (SEQ ID | (SEQ ID |
| | NO: 39) | | | | NO: 51) | NO: 54) |

TABLE 6
Amino Acid Sequences for the 6 CDR Domains in HuMAb 25J60 as Defined using the
Kabat, Chothia and IMGT Methods

| 25J60 | | | | | | |
|---|---|---|---|---|---|---|
| CDR | | | | | | |
| Defini- | Amino Acid Sequences and SEQ ID Nos. | | | | | |
| tions | VH CDR1 | VH CDR2 | VH CDR3 | VL CDR1 | VL CDR2 | VL CDR3 |
| Kabat | RYYMY | ILNPNQDQTTYAQKFQG | TYRYYMDV | RASQSVRSNYLA | GASSRAT | QQYGSSPRT |
| | (SEQ ID | (SEQ ID NO: 58) | (SEQ ID | (SEQ ID NO: 64) | (SEQ ID | (SEQ ID |
| | NO: 55) | | NO: 61) | | NO: 67) | NO: 70) |
| Chothia | GNTQIRY | NPNQDQ | TYRYYMDV | RASQSVRSNYLA | GASSRAT | QQYGSSPRT |
| | (SEQ ID | (SEQ ID NO: 59) | (SEQ ID | (SEQ ID NO: 65) | (SEQ ID | (SEQ ID |
| | NO: 56) | | NO: 62) | | NO: 68) | NO: 71) |
| IMGT | GNTQIRYY | LNPNQDQT | ATTYRYYMDV | QSVRSNY | GAS | QQYGSSPRT |
| | (SEQ ID | (SEQ ID NO: 60) | (SEQ ID | (SEQ ID NO: 66) | (SEQ ID | (SEQ ID |
| | NO: 57) | | NO: 63) | | NO: 69) | NO: 72) |

TABLE 7
Amino Acid Sequences for the 6 CDR Domains in HuMAb 25J80 as Defined
using the Kabat, Chothia and IMGT Methods

| 25J80 | | | | | | |
|---|---|---|---|---|---|---|
| CDR | | | | | | |
| Defini- | Amino Acid Sequences and SEQ ID Nos. | | | | | |
| tions | VH CDR1 | VH CDR2 | VH CDR3 | VL CDR1 | VL CDR2 | VL CDR3 |
| Kabat | RYYMH | IIWPNGDQTTYAQKFQG | TYKYAMDV | RASQSVRSNYLA | GASSRAT | QQYESPPRT |
| | (SEQ ID | (SEQ ID NO: 76) | (SEQ ID | (SEQ ID NO: 82) | (SEQ ID | (SEQ ID |
| | NO: 73) | | NO: 79) | | NO: 85) | NO: 88) |
| Chothia | GRTFIRY | WPNGDQ | TYKYAMDV | RASQSVRSNYLA | GASSRAT | QQYESPPRT |
| | (SEQ ID | (SEQ ID NO: 77) | (SEQ ID | (SEQ ID NO: 83) | (SEQ ID | (SEQ ID |
| | NO: 74) | | NO: 80) | | NO: 86) | NO: 89) |
| IMGT | GRTFIRYY | IWPNGDQT | ATTYKYAMDV | QSVRSNY | GAS | QQYESPPRT |
| | (SEQ ID | (SEQ ID NO: 78) | (SEQ ID | (SEQ ID NO: 84) | (SEQ ID | (SEQ ID |
| | NO: 75) | | NO: 81) | | NO: 87) | NO: 90) |

TABLE 8
Amino Acid Sequences for the 6 CDR Domains in HuMAb 8N42 as Defined using
the Kabat, Chothia and IMGT Methods

| 8N42 | | | | | | |
|---|---|---|---|---|---|---|
| CDR | | | | | | |
| Defini- | Amino Acid Sequences and SEQ ID Nos. | | | | | |
| tions | VH CDR1 | VH CDR2 | VH CDR3 | VL CDR1 | VL CDR2 | VL CDR3 |
| Kabat | IYYWS | EINDEGNTNYNPSLKS | GGTGDIHAFDI | RASQGISKWLA | AASSLQS | QQYNSYPWT |
| | (SEQ ID | (SEQ ID NO: 94) | SEQ ID NO: 97) | (SEQ ID NO: | (SEQ ID | (SEQ ID |
| | NO: 91) | | | 100) | NO: 103) | NO: 106) |
| Chothia | GGSFSIY | NDEGN | GGTGDIHAFDI | RASQGISKWLA | AASSLQS | QQYNSYPWT |
| | (SEQ ID | (SEQ ID NO: 95) | (SEQ ID NO: 98) | (SEQ ID NO: | (SEQ ID | (SEQ ID |
| | NO: 92) | | | 101) | NO: 104) | NO: 107) |
| IMGT | GGSFSIYY | INDEGNT | ARGGTGDIHAFDI | QGISKW | AAS | QQYNSYPWT |
| | (SEQ ID | (SEQ ID NO: 96) | (SEQ ID NO: 99) | (SEQ ID NO: | (SEQ ID | (SEQ ID |
| | NO: 93) | | | 102) | NO: 105) | NO: 108) |

TABLE 9
Amino Acid Sequences for the 6 CDR Domains in HuMAb 4K10 as
Defined using the Kabat, Chothia and IMGT Methods

| 4K10 | | | | | | |
|---|---|---|---|---|---|---|
| CDR | | | | | | |
| Defini- | Amino Acid Sequences and SEQ ID Nos. | | | | | |
| tions | VH CDR1 | VH CDR2 | VH CDR3 | VL CDR1 | VL CDR2 | VL CDR3 |
| Kabat | GYYWS | EISHSGSTNYNPSLKS | ALSRYWYFDL | RASQSASNYLA | DASNRAT | YQRSQWPIS |
| | (SEQ ID | (SEQ ID NO: 112) | (SEQ ID NO: | (SEQ ID NO: | (SEQ ID | (SEQ ID |
| | NO: 109) | | 115) | 118) | NO: 121) | NO: 124) |
| Chothia | GGSFSGY | SHSGS | ALS RYWYFDL | RASQSASNYLA | DASNRAT | YQRSQWPIS |
| | (SEQ ID | (SEQ ID NO: 113) | (SEQ ID NO: | (SEQ ID NO: | (SEQ ID | (SEQ ID |
| | NO: 110) | | 116) | 119) | NO: 122) | NO: 125) |
| IMGT | GGSFSGYY | ISHSGST | ARALSRYWYFDL | RASQSASNYLA | DAS | YQRSQWPIS |
| | (SEQ ID | (SEQ ID NO: 114) | (SEQ ID NO: | (SEQ ID NO: | (SEQ ID | (SEQ ID |
| | NO: 111) | | 117) | 120) | NO: 123) | NO: 126) |

TABLE 10
Amino Acid Sequences for the 6 CDR Domains in Humanized MAb 2L105
as Defined using the Kabat, Chothia and IMGT Methods

| 2L105 | | | | | | |
|---|---|---|---|---|---|---|
| CDR | | | | | | |
| Defini- | Amino Acid Sequences and SEQ ID Nos. | | | | | |
| tions | VH CDR1 | VH CDR2 | VH CDR3 | VL CDR1 | VL CDR2 | VL CDR3 |
| Kabat | SFAIS | VIWTGGGTDYNSALKS | HWYLDV | RSSTGAVSTSNYAN | GANSRAP | ALWFSNHWV |
| | (SEQ ID | (SEQ ID NO: 130) | (SEQ ID | (SEQ ID NO: 136) | (SEQ ID | (SEQ ID |
| | NO: 127) | | NO: 133) | | NO: 139) | NO: 142) |
| Chothia | GISLSSF | WTGGG | HWYLDV | RSSTGAVSTSNYAN | GANSRAP | ALWFSNHWV |
| | (SEQ ID | (SEQ ID NO: 131) | (SEQ ID | (SEQ ID NO: 137) | (SEQ ID | (SEQ ID |
| | NO: 128) | | NO: 134) | | NO: 140) | NO: 143) |
| IMGT | GISLSSFA | IWTGGGT | ASHWYLDV | TGAVSTSNY | GAN | ALWFSNHWV |
| | (SEQ ID | (SEQ ID NO: 132) | (SEQ ID | (SEQ ID NO: 138) | (SEQ ID | (SEQ ID |
| | NO: 129) | | NO: 135) | | NO: 141) | NO: 144) |

TABLE 11
Amino Acid Sequences for the 6 CDR Domains in Humanized MAb
4M60 as Defined using the Kabat, Chothia and IMGT Methods

| 4M60 | | | | | | |
|---|---|---|---|---|---|---|
| CDR | | | | | | |
| Defini- | Amino Acid Sequences and SEQ ID Nos. | | | | | |
| tions | VH CDR1 | VH CDR2 | VH CDR3 | VL CDR1 | VL CDR2 | VL CDR3 |
| Kabat | TYGMS | WINNYSGVS | DYYGSGGWVFDY | KSSQSLLDSEGKTYLN | LVSKLDS | WQGTHFPRT |
| | (SEQ ID | TYADDFKG | (SEQ ID NO: | (SEQ ID NO: 154) | (SEQ ID | (SEQ ID |
| | NO: 145) | (SEQ ID | 151) | | NO: 157) | NO: 160) |
| | | NO: 148) | | | | |
| Chothia | GNTFTTY | NNYSGV | DYYGSGGWVFDY | KSSQSLLDSEGKTYLN | LVSKLDS | WQGTHFPRT |
| | (SEQ ID | (SEQ ID | (SEQ ID NO: | (SEQ ID NO: 155) | (SEQ ID | (SEQ ID |
| | NO: 146) | NO: 149) | 152) | | NO: 158) | NO: 161) |
| IMGT | GNTFTTYG | INNYSGVS | ARDYYGSGGWVFDY | QSLLDSEGKTY | LVS | WQGTHFPRT |
| | (SEQ ID | (SEQ ID | (SEQ ID NO: | (SEQ ID NO: 156) | (SEQ ID | (SEQ ID |
| | NO: 147) | NO: 150) | 153) | | NO: 159) | NO: 162) |

TABLE 12
Amino Acid Sequences for the 6 CDR Domains in MoMAb
2D9 as Defined using the Kabat, Chothia and IMGT Methods

| 2D9 CDR | | | | | | |
|---|---|---|---|---|---|---|
| Defini- | Amino Acid Sequences and SEQ ID Nos. | | | | | |
| tions | VH CDR1 | VH CDR2 | VH CDR3 | VL CDR1 | VL CDR2 | VL CDR3 |
| Kabat | SFAIS | VIWTGGGT | HWYLDV | RSSTGAVSTSNYAN | GANSRAP | ALWFSNHWV |
| | (SEQ ID | DYNSALKS | (SEQ ID | (SEQ ID NO: 172) | (SEQ ID | (SEQ ID |
| | NO: 163) | (SEQ ID | NO: 169) | | NO: 175) | NO: 178) |
| | | NO: 166) | | | | |
| Chothia | GISLSSF | WTGGG | HWYLDV | RSSTGAVSTSNYAN | GANSRAP | ALWFSNHWV |
| | (SEQ ID | (SEQ ID | (SEQ ID | (SEQ ID NO: 173) | (SEQ ID | (SEQ ID |
| | NO: 164) | NO: 167) | NO: 170) | | NO: 176) | NO: 179) |
| IMGT | GISLSSFA | IWTGGGT | ASHWYLDV | TGAVSTSNY | GAN | ALWFSNHWV |
| | (SEQ ID | (SEQ ID | (SEQ ID | (SEQ ID NO: 174) | (SEQ ID | (SEQ ID |
| | NO: 165) | NO: 168) | NO: 171) | | NO: 177) | NO: 180) |

TABLE 13
Amino Acid Sequences for the 6 CDR Domains in MoMAb 4E9
as Defined using the Kabat, Chothia and IMGT Methods

| 4E9 CDR | | | | | | |
|---|---|---|---|---|---|---|
| Defini- | Amino Acid Sequences and SEQ ID Nos. | | | | | |
| tions | VH CDR1 | VH CDR2 | VH CDR3 | VL CDR1 | VL CDR2 | VL CDR3 |
| Kabat | TYGMS | WINNYSGVSTYADDFKG | DYYGSGGWVFDY | KSSQSLLDSDGKTYLN | LVSKLDS | WQGTHFPRT |
| | (SEQ ID | (SEQ ID NO: 184) | (SEQ ID NO: 187) | (SEQ ID NO: 190) | (SEQ ID | (SEQ ID |
| | NO: 181) | | | | NO: 193) | NO: 196) |
| Chothia | GNTFTTY | NNYSGV | DYYGSGGWVFDY | KSSQSLLDSDGKTYLN | LVSKLDS | WQGTHFPRT |
| | (SEQ ID | (SEQ ID NO: 185) | (SEQ ID NO: 188) | (SEQ ID NO: 191) | (SEQ ID | (SEQ ID |
| | NO: 182) | | | | NO: 194) | NO: 197) |
| IMGT | GNTFTTYG | INNYSGVS | ARDYYGSGGWVFDY | QSLLDSDGKTY | LVS | WQGTHFPRT |
| | (SEQ ID | (SEQ ID NO: 186) | (SEQ ID NO: 189) | (SEQ ID NO: 192) | (SEQ ID | (SEQ ID |
| | NO: 183) | | | | NO: 195) | NO: 198) |

TABLE 14
Amino Acid Sequences for the 6 CDR Domains in MoMAb
16B9 as Defined using the Kabat, Chothia and IMGT Methods

| 16B9 | | | | | | |
|---|---|---|---|---|---|---|
| CDR | | | | | | |
| Defini- | Amino Acid Sequences and SEQ ID Nos. | | | | | |
| tions | VH CDR1 | VH CDR2 | VH CDR3 | VL CDR1 | VL CDR2 | VL CDR3 |
| Kabat | DYNMH | YIHPNNGGTSYNQKFKD | SGIYYDYDSFFDY | RASENIYSHLA | AATNLAD | QHFWGSPWT |
| | (SEQ ID | (SEQ ID NO: 202) | (SEQ ID NO: 205) | (SEQ ID NO: | (SEQ ID | (SEQ ID |
| | NO: 199) | | | 208) | NO: 211) | NO: 214) |
| Chothia | GYTFIDY | HPNNGG | SGIYYDYDSFFDY | RASENIYSHLA | AATNLAD | QHFWGSPWT |
| | (SEQ ID | (SEQ ID NO: 203) | (SEQ ID NO: 206) | (SEQ ID NO: | (SEQ ID | (SEQ ID |
| | NO: 200) | | | 209) | NO: 212) | NO: 215) |
| IMGT | GYTFIDYN | IHPNNGGT | SRSGIYYDYDSFFDY | ENIYSH | AAT | QHFWGSPWT |
| | (SEQ ID | (SEQ ID NO: 204) | (SEQ ID NO: 207) | (SEQ ID NO: | (SEQ ID | (SEQ ID |
| | NO: 201) | | | 210) | NO: 213) | NO: 216) |

TABLE 15
Amino Acid Sequences for the V_{H}, V_{L}, Heavy Chain and Light Chain in HuMAb 1B4

| mAb | |
|---|---|
| 1B4 | Amino Acid Sequences and SEQ ID Nos. |
| V_{H} | QLQLQESGPGLVKPSETLSLTCAVSGYSISSGNYWGWIRQSPGKGLEWIGSVDHSGSTYYSPSLKSRVTISVDTSKNQFSLKLNSVTAADTADYY |
| | CARNTMIRGVMDWFDPWGQGTLVTVSS (SEQ ID NO: 217) |
| V_{L} | AIQLTQSPSSLSASVGDRVTITCRASQGISSALAWYQQKPGKAPKVLIYDASSLESGVPSRFSGSGSGTDFTLTISSLQPEDFATYYCQQFRSYP |
| | TFGQGTKVEIK (SEQ ID NO: 218) |
| Heavy | QLQLQESGPGLVKPSETLSLTCAVSGYSISSGNYWGWIRQSPGKGLEWIGSVDHSGSTYYSPSLKSRVTISVDTSKNQFSLKLNSVTAADTADYY |
| Chain | CARNTMIRGVMDWFDPWGQGTLVTVSSASTKGPSVFPLAPSSKSTSGGTAALGCLVKDYFPEPVTVSWNSGALTSGVHTFPAVLQSSGLYSLSSV |
| | VTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCSVMHEALHNHYTQKSLSLSPGK (SEQ ID NO: 219) |
| Light | AIQLTQSPSSLSASVGDRVTITCRASQGISSALAWYQQKPGKAPKVLIYDASSLESGVPSRFSGSGSGTDFTLTISSLQPEDFATYYCQQFRSYP |
| Chain | TFGQGTKVEIKRTVAAPSVFIFPPSDEQLKSGTASVVCLLNNFYPREAKVQWKVDNALQSGNSQESVTEQDSKDSTYSLSSTLTLSKADYEKHKV |
| | YACEVTHQGLSSPVTKSFNRGEC (SEQ ID NO: 220) |

TABLE 16
Amino Acid Sequences for the V_{H}, V_{L}, Heavy Chain and Light Chain in HuMAb 10K11

| mAb | |
|---|---|
| 10K11 | Amino Acid Sequences and SEQ ID Nos. |
| V_{H} | QLQLQESGPGLVKPSETLSLTCAVSGYSISSDYSWGWIRQPPGKGLEWIGSIYHSGNTYFNPSLKSRVTISVDTSKNQFSLKLSSVTAADTAVYY |
| | CARDKSYYFGPGSMDVWGQGTTVTVSS (SEQ ID NO: 221) |
| V_{L} | AIQLTQSPSSLSASVGDRVTITCRASQGISSALAWYQQKPGKAPKLLIYDASSLESGVPSRFSGSGSGTDFTLTISSLQPEDFATYYCQQFKSYL |
| | TFGQGTRLEIK (SEQ ID NO: 222) |
| Heavy | QLQLQESGPGLVKPSETLSLTCAVSGYSISSDYSWGWIRQPPGKGLEWIGSIYHSGNTYFNPSLKSRVTISVDTSKNQFSLKLSSVTAADTAVYY |
| Chain | CARDKSYYFGPGSMDVWGQGTTVTVSASTKGPSVFPLAPSSKSTSGGTAALGCLVKDYFPEPVTVSWNSGALTSGVHTFPAVLQSSGLYSLSSV |
| | VTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKTISKAKGQPREPQVYTLPPSREEMTKNQVSLTCLVKGFYPS |
| | DIAVEWESNGQPENNYKTTPPVLDSDGSFFLYSKLTVDKSRWQQGNVFSCSVMHEALHNHYTQKSLSLSPGK (SEQ ID NO: 223) |
| Light | AIQLTQSPSSLSASVGDRVTITCRASQGISSALAWYQQKPGKAPKLLIYDASSLESGVPSRFSGSGSGTDFTLTISSLQPEDFATYYCQQFKSYL |
| Chain | TFGQGTRLEIKRTVAAPSVFIFPPSDEQLKSGTASVVCLLNNFYPREAKVQWKVDNALQSGNSQESVTEQDSKDSTYSLSSTLTLSKADYEKHKV |
| | YACEVTHQGLSSPVTKSFNRGEC (SEQ ID NO: 224) |

TABLE 17
Amino Acid Sequences for the V_{H}, V_{L}, Heavy Chain and Light Chain in HuMAb 22I16

| mAb | |
|---|---|
| 22I16 | Amino Acid Sequences and SEQ ID Nos. |
| V_{H} | EVQLVESGGGLVQPGGSLRLSCAASGFTFSSYSMNWVRQAPGKGLEWVSYIISGSDTIFYADSVKGRFTISRDNAKNSLYLQMNSLRDEDTAVYY |
| | CARDETVVRGVINYFDYWGQGTLVTVSS (SEQ ID NO: 225) |
| V_{L} | AIQLTQSPSSLSASVGDRVTITCRSSQGISSALAWYQQKPGKAPKLLIYDASSLESGVPSRFSGSGSGTDFTLTISSLQPEDFATYYCQQFISYP |
| | TFGQGTRLEIK (SEQ ID NO: 226) |
| Heavy | EVQLVESGGGLVQPGGSLRLSCAASGFTFSSYSMNWVRQAPGKGLEWVSYIISGSDTIFYADSVKGRFTISRDNAKNSLYLQMNSLRDEDTAVYY |
| Chain | CARDETVVRGVINYFDYWGQGTLVTVSSASTKGPSVFPLAPSSKSTSGGTAALGCLVKDYFPEPVTVSWNSGALTSGVHTFPAVLQSSGLYSLSS |
| | VVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEKTISKAKGQPREPQVYTLPPSREEMTKNQVSLTCLVKGFYP |
| | SDIAVEWESNGQPENNYKTTPPVLDSDGSFFLYSKLTVDKSRWQQGNVFSCSVMHEALHNHYTQKSLSLSPGK (SEQ ID NO: 227) |
| Light | AIQLTQSPSSLSASVGDRVTITCRSSQGISSALAWYQQKPGKAPKLLIYDASSLESGVPSRFSGSGSGTDFTLTISSLQPEDFATYYCQQFISYP |
| Chain | TFGQGTRLEIKRTVAAPSVFIFPPSDEQLKSGTASVVCLLNNFYPREAKVQWKVDNALQSGNSQESVTEQDSKDSTYSLSSTLTLSKADYEKHKV |
| | YACEVTHQGLSSPVTKSFNRGEC (SEQ ID NO: 228) |

TABLE 18
Amino Acid Sequences for the V_{H}, V_{L}, Heavy Chain and Light Chain in HuMAb 25J60

| mAb | |
|---|---|
| 25J60 | Amino Acid Sequences and SEQ ID Nos. |
| V_{H} | QVQLVQSGAEVKKPGASVKVSCKTSGNTQIRYYMYWVRQAPGQGLEWMGILNPNQDQTTYAQKFQGRVTMTRDTSTSTVYMELSSLRSEDTAVYY |
| | CATTYRYYMDVWGQGTTVTVSS (SEQ ID NO: 229) |
| V_{L} | EIVLTQSPGTLSLSPGERATLSCRASQSVRSNYLAWYQQKPGQAPRLLIYGASSRATGIPDRFSGSGSGTDFTLTISRLEPEDFAVYYCQQYGSS |
| | PRTFGQGTKVEIK (SEQ ID NO: 230) |
| Heavy | QVQLVQSGAEVKKPGASVKVSCKTSGNTQIRYYMYWVRQAPGQGLEWMGILNPNQDQTTYAQKFQGRVTMTRDTSTSTVYMELSSLRSEDTAVYY |
| Chain | CATTYRYYMDVWGQGTTVTVSSASTKGPSVFPLAPSSKSTSGGTAALGCLVKDYFPEPVTVSWNSGALTSGVHTFPAVLQSSGLYSLSSVVTVPS |
| | SSLGTQTYTCNVNHKPSNTKVDKRVEPKSCDKTHTCPPCPAPEAEGAPSVFLFPPKPKDTLMISRTPEVICVVVDVSHEDPEVKFNWYVDGVEVH |
| | NAKTKPREEQYNSTYRVVSVLTVLHQDWLNGKEYKCKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYTQKSLSLSPGK (SEQ ID NO: 231) |
| Light | EIVLTQSPGTLSLSPGERATLSCRASQSVRSNYLAWYQQKPGQAPRLLIYGASSRATGIPDRFSGSGSGTDFTLTISRLEPEDFAVYYCQQYGSS |
| Chain | PRTFGQGTKVEIKRTVAAPSVFIFPPSDEQLKSGTASVVCLLNNFYPREAKVQWKVDNALQSGNSQESVTEQDSKDSTYSLSSTLTLSKADYEKH |
| | KVYACEVTHQGLSSPVTKSFNRGEC (SEQ ID NO: 232) |

TABLE 19
Amino Acid Sequences for the V_{H}, V_{L}, Heavy Chain and Light Chain in HuMAb 25J80

| mAb | |
|---|---|
| 25J80 | Amino Acid Sequences and SEQ ID Nos. |
| V_{H} | QVQLVQSGAEVKKPGASVKVSCKTSGRTFIRYYMHWVRQAPGQGLEWMGIIWPNGDQTTYAQKFQGRVTMTRDTSTSTVYMELSSLRSEDTAVYY |
| | CATTYKYAMDVWGQGTTVTVSS (SEQ ID NO: 233) |
| V_{L} | EIVLTQSPGTLSLSPGERATLSCRASQSVRSNYLAWYQQKPGQAPRLLIYGASSRATGIPDRFSGSGSGTDFTLTISRLEPEDFAVYYCQQYESP |
| | PRTFGQGTKVEIK (SEQ ID NO: 234) |
| Heavy | QVQLVQSGAEVKKPGASVKVSCKTSGRTFIRYYMHWVRQAPGQGLEWMGIIWPNGDQTTYAQKFQGRVTMTRDTSTSTVYMELSSLRSEDTAVYY |
| Chain | CATTYKYAMDVWGQGTTVTVSSASTKGPSVFPLAPSSKSTSGGTAALGCLVKDYFPEPVTVSWNSGALTSGVHTFPAVLQSSGLYSLSSVVTVPS |
| | SSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVH |
| | NAKTKPREEQYNSTYRVVSVLTVLHQDWLNGKEYKCKVSNKALPAPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYTQKSLSLSPGK (SEQ ID NO: 235) |
| Light | EIVLTQSPGTLSLSPGERATLSCRASQSVRSNYLAWYQQKPGQAPRLLIYGASSRATGIPDRFSGSGSGTDFTLTISRLEPEDFAVYYCQQYESP |
| Chain | PRTFGQGTKVEIKRTVAAPSVFIFPPSDEQLKSGTASVVCLLNNFYPREAKVQWKVDNALQSGNSQESVTEQDSKDSTYSLSSTLTLSKADYEKH |
| | KVYACEVTHQGLSSPVTKSFNRGEC (SEQ ID NO: 236) |

TABLE 20
Amino Acid Sequences for the V_{H}, V_{L}, Heavy Chain and Light Chain in HuMAb 8N42

| mAb | |
|---|---|
| 8N42 | Amino Acid Sequences and SEQ ID Nos. |
| V_{H} | QVQLQQWGAGLLKPSETLSLTCAVYGGSFSIYYWSWIRQPPGKGLELIGEINDEGNTNYNPSLKSRVTISVDTSKNQFSLKLSSVTAADTAVYYC |
| | ARGGTGDIHAFDIWGQGTMVTVSS (SEQ ID NO: 237) |
| V_{L} | DIQMTQSPSSLSASVGDRVTITCRASQGISKWLAWYQQKPEKAPKSLIYAASSLQSGVPSRFSGSGSGTDFTLTISSLQPEDFATYYCQQYNSYP |
| | WTFGQGTKVEIK (SEQ ID NO: 238) |
| Heavy | QVQLQQWGAGLLKPSETLSLTCAVYGGSFSIYYWSWIRQPPGKGLELIGEINDEGNTNYNPSLKSRVTISVDTSKNQFSLKLSSVTAADTAVYYC |
| Chain | ARGGTGDIHAFDIWGQGTMVTVSSASTKGPSVFPLAPSSKSTSGGTAALGCLVKDYFPEPVTVSWNSGALTSGVHTFPAVLQSSGLYSLSSVVTV |
| | PSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALPAPIEKTISKAKGQPREPQVYTLPPSREEMTKNQVSLTCLVKGFYPSDIA |
| | VEWESNGQPENNYKTTPPVLDSDGSFFLYSKLTVDKSRWQQGNVFSCSVMHEALHNHYTQKSLSLSPGK (SEQ ID NO: 239) |
| Light | DIQMTQSPSSLSASVGDRVTITCRASQGISKWLAWYQQKPEKAPKSLIYAASSLQSGVPSRFSGSGSGTDFTLTISSLQPEDFATYYCQQYNSYP |
| Chain | WTFGQGTKVEIKRTVAAPSVFIFPPSDEQLKSGTASVVCLLNNFYPREAKVQWKVDNALQSGNSQESVTEQDSKDSTYSLSSTLTLSKADYEKHK |
| | VYACEVTHQGLSSPVTKSFNRGEC (SEQ ID NO: 240) |

TABLE 21
Amino Acid Sequences for the V_{H}, V_{L}, Heavy Chain and Light Chain in HuMAb 4K10

| mAb | |
|---|---|
| 4K10 | Amino Acid Sequences and SEQ ID Nos. |
| V_{H} | QVQLQQWGAGLLKPSETLSLTCAVYGGSFSGYYWSWLRQPPGKGLEWIGEISHSGSTNYNPSLKSRVTISVDTSKNQFSLKLSSVTAADTAVYYC |
| | ARALSRYWYFDLWGRGTLVTVSS (SEQ ID NO: 241) |
| V_{L} | EIVLTQSPATLSLSPGERATLSCRASQSASNYLAWYQQKPGQAPRLLIYDASNRATGIPARFSGSGSGTDFTLTISSLEPEDFAVYYCYQRSQWP |
| | ISFGQGTRLEIK (SEQ ID NO: 242) |
| Heavy | QVQLQQWGAGLLKPSETLSLTCAVYGGSFSGYYWSWLRQPPGKGLEWIGEISHSGSTNYNPSLKSRVTISVDTSKNQFSLKLSSVTAADTAVYYC |
| Chain | ARALSRYWYFDLWGRGTLVTVSSASTKGPSVFPLAPSSKSTSGGTAALGCLVKDYFPEPVTVSWNSGALTSGVHTFPAVLQSSGLYSLSSVVTVP |
| | SSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKGQPREPQVYTLPPSREEMTKNQVSLTCLVKGFYPSDIAV |
| | EWESNGQPENNYKTTPPVLDSDGSFFLYSKLTVDKSRWQQGNVFSCSVMHEALHNHYTQKSLSLSPGK (SEQ ID NO: 243) |
| Light | EIVLTQSPATLSLSPGERATLSCRASQSASNYLAWYQQKPGQAPRLLIYDASNRATGIPARFSGSGSGTDFTLTISSLEPEDFAVYYCYQRSQWP |
| Chain | ISFGQGTRLEIKRTVAAPSVFIFPPSDEQLKSGTASVVCLLNNFYPREAKVQWKVDNALQSGNSQESVTEQDSKDSTYSLSSTLTLSKADYEKHK |
| | VYACEVTHQGLSSPVTKSFNRGEC (SEQ ID NO: 244) |

TABLE 22
Amino Acid Sequences for the V_{H}, V_{L}, Heavy Chain and Light Chain in Humanized MAb 2L105

| mAb | |
|---|---|
| 2L105 | Amino Acid Sequences and SEQ ID Nos. |
| V_{H} | QVTLKESGPVLVKPTETLTLTCTVSGISLSSFAISWIRQPPGKALEWLAVIWTGGGTDYNSALKSRLTISKDTSKSQVVLTMTNMDPVDTATYYC |
| | ASHWYLDVWGQGTTVTVSS (SEQ ID NO: 245) |
| V_{L} | QTVVTQEPSFSVSPGGTVTLTCRSSTGAVSTSNYANWVQQTPGQAPRGLIGGANSRAPGIPDRFSGSILGNKAALTITGAQADDESDYYCALWFS |
| | NHWVFGGGTKLTVL (SEQ ID NO: 246) |
| Heavy | QVTLKESGPVLVKPTETLTLTCTVSGISLSSFAISWIRQPPGKALEWLAVIWTGGGTDYNSALKSRLTISKDTSKSQVVLTMTNMDPVDTATYYC |
| Chain | ASHWYLDVWGQGTTVTVSSASTKGPSVFPLAPSSKSTSGGTAALGCLVKDYFPEPVTVSWNSGALTSGVHTFPAVLQSSGLYSLSSVVTVPSSSL |
| | GTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALPAPIEKTISKAKGQPREPQVYTLPPSREEMTKNQVSLTCLVKGFYPSDIAVEWES |
| | NGQPENNYKTTPPVLDSDGSFFLYSKLTVDKSRWQQGNVFSCSVMHEALHNHYTQSLSLSPGK (SEQ ID NO: 247) |
| Light | QTVVTQEPSFSVSPGGTVTLTCRSSTGAVSTSNYANWVQQTPGQAPRGLIGGANSRAPGIPDRFSGSILGNKAALTITGAQADDESDYYCALWFS |
| Chain | NHWVFGGGTKLTVLGQPKAAPSVTLFPPSSEELQANKATLVCLISDFYPGAVTVAWKADSSPVKAGVETTTPSKQSNNKYAASSYLSLTPEQWKS |
| | HRSYSCQVTHEGSTVEKTVAPTECS (SEQ ID NO: 248) |

TABLE 23
Amino Acid Sequences for the V_{H}, V_{L}, Heavy Chain and Light Chain in Humanized mAb 4M60

| mAb | |
|---|---|
| 4M60 | Amino Acid Sequences and SEQ ID Nos. |
| V_{H} | QVQLVQSGSELKKPGASVKVSCKASGNTFTTYGMSWVRQAPGQGLEWMGWINNYSGVSTYADDFKGRFVFSLDTSVSTAYLQISSLKAEDTAVYY |
| | CARDYYGSGGWVFDYWGQGTTVTVSS (SEQ ID NO: 249) |
| V_{L} | DVVMTQSPLSLPVTLGQPASISCKSSQSLLDSEGKTYLNWLQQRPGQSPRRLMYLVSKLDSGVPDRFSGSGSGTDFTLKISRVEAEDVGVYYCWQ |
| | GTHFPRTFGGGTKVEIK (SEQ ID NO: 250) |
| Heavy | QVQLVQSGSELKKPGASVKVSCKASGNTFTTYGMSWVRQAPGQGLEWMGWINNYSGVSTYADDFKGRFVFSLDTSVSTAYLQISSLKAEDTAVYY |
| Chain | CARDYYGSGGWVFDYWGQGTTVTVSSASTKGPSVFPLAPSSKSTSGGTAALGCLVKDYFPEPVTVSWNSGALTSGVHTFPAVLQSSGLYSLSSVV |
| | TVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTISKAKGQPREPQVYTLPPSREEMTKNQVSLTCLVKGFYPSD |
| | IAVEWESNGQPENNYKTTPPVLDSDGSFFLYSKLTVDKSRWQQGNVFSCSVMHEALHNHYTQKSLSLSPGK (SEQ ID NO: 251) |
| Light | DVVMTQSPLSLPVTLGQPASISCKSSQSLLDSEGKTYLNWLQQRPGQSPRRLMYLVSKLDSGVPDRFSGSGSGTDFTLKISRVEAEDVGVYYCWQ |
| Chain | GTHFPRTFGGGTKVEIKRTVAAPSVFIFPPSDEQLKSGTASVVCLLNNFYPREAKVQWKVDNALQSGNSQESVTEQDSKDSTYSLSSTLTLSKAD |
| | YEKHKVYACEVTHQGLSSPVTKSFNRGEC (SEQ ID NO: 252) |

TABLE 24
Amino Acid Sequences
for the V_{H} and V_{L} Regions in MoMAb 2D9

| mAb | |
|---|---|
| 2D9 | Amino Acid Sequences and SEQ ID Nos. |
| V_{H} | QVQLKESGPGLVAPSQSLSITCTVSGISLSSFAISWVRQPPGKGL |
| | EWLGVIWTGGGTDYNSALKSRLTISKDTSKNQVFLKMNSLQTDDT |
| | ARYYCASHWYLDVWGTGTTVTVSS (SEQ ID NO: 253) |
| V_{L} | QAVVTQESALTTSPGETVTLTCRSSTGAVSTSNYANWVQEKPDHL |
| | FTGLIGGANSRAPGIPARFSGSLIGDKAALTITGAQTEDEAIYFC |
| | ALWFSNHWVFGGGTKLTVL (SEQ ID NO: 254) |

TABLE 25
Amino Acid Sequences
for the V_{H} and V_{L} Regions in MoMAb 4E9

| mAb | |
|---|---|
| 4E9 | Amino Acid Sequences and SEQ ID Nos. |
| V_{H} | QIQLVQSGPELKKPGETVKISCKASGNTFTTYGMSWVKQAPGKNL |
| | KWMGWINNYSGVSTYADDFKGRFAFSLETSATTAYLQINNLTNED |
| | SATYFCARDYYGSGGWVFDYWGQGTTLTVSS (SEQ ID NO: |
| | 255) |
| V_{L} | DVVMTQTPLTLSVTIGQPASISCKSSQSLLDSDGKTYLNWLLQRP |
| | GQSPKRLMYLVSKLDSGVPDRFTGSGSGTDFTLKISRVEAEDLGV |
| | YYCWQGTHFPRTFGGGTKLEIK (SEQ ID NO: 256) |

TABLE 26
Amino Acid Sequences
for the V_{H} and V_{L} Regions in MoMAb 16B9

| mAb | |
|---|---|
| 16B9 | Amino Acid Sequences and SEQ ID Nos. |
| V_{H} | EVQLQQSRPDLVKPGASVKMSCKASGYTFIDYNMHWVKQRHGK |
| | SLEWIGYIHPNNGGTSYNQKFKDKATLTMNKSSSTAYMELRSL |
| | TSEDSAVYYCSRSGIYYDYDSFFDYWGQGTTLTVSS (SEQ |
| | ID NO: 257) |
| V_{L} | DIQMTQSPASLYVSVGETVTITCRASENIYSHLAWYQQKLGKS |
| | PHLLVYAATNLADGVPSRFSGSGSGTQYSLKINSLQSEDFGSY |
| | YCQHFWGSPWTFGGGTKLEIK (SEQ ID NO: 258) |

TABLE 27
Amino Acid Sequences for the Human MerTK Polypeptide

| MerTK | Amino Acid Sequences and SEQ ID Nos. |
|---|---|
| Human | 1 MGPAPLPLLL GLFLPALWRR AITEAREEAK PYPLFPGPFP GSLQTDHTPL LSLPHASGYQ |
| | 61 PALMFSPTQP GRPHTGNVAI PQVTSVESKP LPPLAFKHTV GHIILSEHKG VKFNCSISVP |
| | 121 NIYQDTTISW WKDGKELLGA HHAITQFYPD DEVTAIIASF SITSVQRSDN GSYICKMKIN |
| | 181 NEEIVSDPIY IEVQGLPHFT KQPESMNVTR NTAFNLTCQA VGPPEPVNIF WVQNSSRVNE |
| | 241 QPEKSPSVLT VPGLTEMAVF SCEAHNDKGL TVSKGVQINI KAIPSPPTEV SIRNSTAHSI |
| | 301 LISWVPGFDG YSPFRNCSIQ VKEADPLSNG SVMIFNTSAL PHLYQIKQLQ ALANYSIGVS |
| | 361 CMNEIGWSAV SPWILASTTE GAPSVAPLNV TVFLNESSDN VDIRWMKPPT KQQDGELVGY |
| | 421 RISHVWQSAG ISKELLEEVG QNGSRARISV QVHNATCTVR IAAVTRGGVG PFSDPVKIFI |
| | 481 PAHGWVDYAP SSTPAPGNAD PVLIIFGCFC GFILIGLILY ISLAIRKRVQ ETKFGNAFTE |
| | 541 EDSELVVNYI AKKSFCRRAI ELTLHSLGVS EELQNKLEDV VIDRNLLILG KILGEGEFGS |
| | 601 VMEGNLKQED GTSLKVAVKT MKLDNSSQRE IEEFLSEAAC MKDFSHPNVI RLLGVCIEMS |
| | 661 SQGIPKPMVI LPFMKYGDLH TYLLYSRLET GPKHIPLQTL LKFMVDIALG MEYLSNRNFL |
| | 721 HRDLAARNCM LRDDMTVCVA DFGLSKKIYS GDYYRQGRIA KMPVKWIAIE SLADRVYTSK |
| | 781 SDVWAFGVTM WEIATRGMTP YPGVQNHEMY DYLLHGHRLK QPEDCLDELY EIMYSCWRTD |
| | 841 PLDRPTFSVL RLQLEKLLES LPDVRNQADV IYVNTQLLES SEGLAQGSTL APLDLNIDPD |
| | 901 SIIASCTPRA AISVVTAEVH DSKPHEGRYI LNGGSEEWED LTSAPSAAVT AEKNSVLPGE |
| | 961 RLVRNGVSWS HSSMLPLGSS LPDELLFADD SSEGSEVLM (SEQ ID NO: 259) |

The complete hMerTK, cMerTK and mMerTK amino acid sequences can be found under GENBANK® Accession Nos. NP_006334.2, XP_005575320.1 and NP_032613.1, respectively.

TABLE 28
Amino Acid Sequences for the Cynomolgus Monkey MerTK Polypeptide

| MerTK | Amino Acid Sequences and SEQ ID Nos. |
|---|---|
| Cynomolgus | 1 MGLAPLPLPL LLGLFLPALW SRAITEAREE AKPYPLFPGP LPGSLQTDHT SLLSLPHTSG |
| | 61 YQPALMFSPT QPGRPYTGNV AIPRVTSAGS KLLPPLAFKH TVGHIILSEH KDVKFNCSIS |
| | 121 VPNIYQDTTI SWWKDGKELL GAHHAITQFY PDDEVTAIIA SFSITSVQRS DNGSYICKMK |
| | 181 INNEEIVSDP IYIEVQGLPH FTKQPESMNV TRNTAFNLTC QAVGPPEPVN IFWVQNSSRV |
| | 241 NEQPEKSPSV LTVPGLTEMA VFSCEAHNDK GLTVSKGVQI NIKAIPSPPT EVSIHNSTAH |
| | 301 SILISWVPGF DGYSPFRNCS VQVKEVDPLS NGSVMIFNTS ASPHMYQIKQ LQALANYSIG |
| | 361 VSCMNEIGWS AVSPWILAST TEGAPSVAPL NVTVFLNESR DNVDIRWMKP LTKRQAGELV |
| | 421 GYRISHVWQS AGISKELLEE VGQNNSRAQI SVQVHNATCT VRIAAVTKGG VGPFSDPVKI |
| | 481 FIPAHGWVDH APSSTPAPGN ADPVLIIFGC FCGFILIGLV LYISLAVRKR VQETKFGNAF |
| | 541 TEEDSELVVN YIAKKSFCRR AIELTLHSLG VSEELQNKLE DVVIDRNLLI LGKILGEGEF |
| | 601 GSVMEGNLKQ EDGTSQKVAV KTMKLDNFSQ REIEEFLSEA ACMKDFSHPN VIRLLGVCIE |
| | 661 MSSQGIPKPM VILPFMKYGD LHTYLLYSRL ETGPKHIPLQ TLLKFMMDIA LGMEYLSNRN |
| | 721 FLHRDLAARN CMLRDDMTVC VADFGLSKKI YSGDYYRQGR IAKMPVKWIA IESLADRVYT |
| | 781 SKSDVWAFGV TMWEIATRGM TPYPGVQNHE MYDYLLHGHR LKQPEDCLDE LYEIMYSCWR |
| | 841 TDPLDRPTFS VLRLQLEKLL ESLPNVRNQA DVIYINTQLL ESSEGLAEGS TLAPLDLNID |
| | 901 PDSIIASCSP HAAISVVTAE IHDSKPHEGR YILNGGSEEW EDVTSAASAA VTAEKNSVLP |
| | 961 GERLVRNGVP WSHSSTLPLG SSLPDELLFA DDSSESSEVL M (SEQ ID NO: 260) |

TABLE 29
Amino Acid Sequences for the Mouse MerTK Polypeptide

| MerTK | Amino Acid Sequences and SEQ ID Nos. |
|---|---|
| Mouse | 1 MVLAPLLLGL LLLPALWSGG TAEKWEETEL DQLFSGPLPG RLPVNHRPFS APHSSRDQLP |
| | 61 PPQTGRSHPA HTAAPQVTST ASKLLPPVAF NHTIGHIVLS EHKNVKFNCS INIPNTYQET |
| | 121 AGISWWKDGK ELLGAHHSIT QFYPDEEGVS IIALFSIASV QRSDNGSYFC KMKVNNREIV |
| | 181 SDPIYVEVQG LPYFIKQPES VNVTRNTAFN LTCQAVGPPE PVNIFWVQNS SRVNEKPERS |
| | 241 PSVLTVPGLT ETAVFSCEAH NDKGLTVSKG VHINIKVIPS PPTEVHILNS TAHSILVSWV |
| | 301 PGFDGYSPLQ NCSIQVKEAD RLSNGSVMVF NTSASPHLYE IQQLQALANY SIAVSCRNEI |
| | 361 GWSAVSPWIL ASTTEGAPSV APLNITVFLN ESNNILDIRW TKPPIKRQDG ELVGYRISHV |
| | 421 WESAGTYKEL SEEVSQNGSW AQIPVQIHNA TCTVRIAAIT KGGIGPFSEP VNIIIPEHSK |
| | 481 VDYAPSSTPA PGNTDSMFII LGCFCGFILI GLILCISLAL RRRVQETKFG GAFSEEDSQL |
| | 541 VVNYRAKKSF CRRAIELTLQ SLGVSEELQN KLEDVVIDRN LLVLGKVLGE GEFGSVMEGN |
| | 601 LKQEDGTSQK VAVKTMKLDN FSQREIEEFL SEAACMKDFN HPNVIRLLGV CIELSSQGIP |
| | 661 KPMVILPFMK YGDLHTFLLY SRLNTGPKYI HLQTLLKFMM DIAQGMEYLS NRNFLHRDLA |
| | 721 ARNCMLRDDM TVCVADFGLS KKIYSGDYYR QGRIAKMPVK WIAIESLADR VYTSKSDVWA |
| | 781 FGVTMWEITT RGMTPYPGVQ NHEMYDYLLH GHRLKQPEDC LDELYDIMYS CWSADPLDRP |
| | 841 TFSVLRLQLE KLSESLPDAQ DKESIIYINT QLLESCEGIA NGPSLTGLDM NIDPDSIIAS |
| | 901 CTPGAAVSVV TAEVHENNLR EERYILNGGN EEWEDVSSTP FAAVTPEKDG VLPEDRLTKN |
| | 961 GVSWSHHSTL PLGSPSPDEL LFVDDSLEDS EVLM (SEQ ID NO: 261) |

Functional Screening for Antagonistic Anti MerTK moMAbs

These moMAb clones were screened using assays to measure inhibition of efferocytosis and inhibition of Gas6-mediated signaling, and counter-screened for agonist potential, as described in Example 2. Clones were selected for further characterization on the bases of: binding to MerTK on human and/or mouse cells (tumor cell lines and primary cells) with sub-nanomolar EC₅₀; and inhibiting efferocytosis to more than 80% of the maximal signal with sub-nanomolar IC₅₀; and inhibiting Gas6-mediated signaling by more than 80% of control with sub-nanomolar IC₅₀ and no agonistic capacity. DNA encoding the Ab variable regions in these clones was sequenced by next generation sequencing and about 200 clones were selected based on potency in inhibiting efferocytosis and MerTK-mediated signaling, sequence diversity and limited potential sequence liabilities. Three moMAbs showed potent antagonistic activity, i.e., IC₅₀ values less than 10 nM in the signaling assay, and were selected for further analysis.

Characterization of Binding Affinity, Binding Kinetics and Binning of Anti-MerTK moMAbs

The affinities and binding kinetics of the three selected moMAbs against mouse, human and cynomolgus monkey MerTK were characterized by SPR analysis. Two of these Abs, 2D9 and 4E9, showed potent antagonistic activity and bound with high affinity to mouse, human and cynomolgus monkey MerTK, whereas the third selected moMAb, 16B9, bound to mMerTK but not to human or cynomolgus monkey MerTK, indicating that mAb 16B9 bound to a different epitope than the one bound by 2D9 or 4 E9. SPR binding competition studies to identify mAbs that compete for the same or overlapping epitope on hMerTK antigen assigned both 2D9 and 4E9 to Bin 2.

Humanized variants of both 2D9 and 4E9 were generated. Binning data and the results of the efferocytosis and signaling assays for 2D9, 4E9 and 16B9, as well as for humanized Abs 2L105 and 4M60 which were generated from moMAbs 2D9 and 4E9, respectively, are included in Table 1.

The binding kinetics data obtained for the selected moMAbs and their humanized versions are included in Table 2.

The sequences for the 6 CDRs for humanized mAbs 2L105 and 4M60 are shown in Tables 10 and 11, respectively, while the sequences for the 6 CDRs for moMAbs 2D9, 4E9 and 16B9 are shown in Tables 12-14, respectively.

The amino acid sequences for the V_{H}, V_{L}, heavy chain and light chain for humanized mAbs 2L107 and 4M60 are shown in Tables 22 and 23, respectively, and the sequences for the V_{H} and V_{L} regions for moMAbs 2D9, 4E9 and 16B9 are shown in Tables 24-26, respectively.

The amino acid sequences for the human, cynomolgus monkey and mouse MerTK polypeptides are shown in Tables 27-29, respectively.

### Example 4

### Anti-MERTK Enhances Anti-Tumor Activity of Anti-PD-1 in MC38 Tumor Model

The anti-tumor activity of the mouse anti-MerTK mAb, 4E9 (mouse IgG1 isotype), was assessed in combination with an anti-mouse PD-1 Ab, 4H2, in a MC38 colon adenocarcinoma mouse tumor model. 4H2 is a chimeric rat-mouse anti-mPD-1 Ab constructed from a rat IgG2a anti-mouse PD-1 Ab in which the Fc-portion was replaced with an Fc-portion from a mouse IgG1 isotype (WO 2006/121168). It blocks binding of mPD-L1 and mPD-L2 binding to mPD-1, stimulates a T cell response, and exhibits anti-tumor activity in a variety of mouse tumor models.

C57BL/6 mice were each injected SC with 10⁶ MC38 tumor cells. Mice were randomized into treatment groups (10 mice/group) after 6 days when tumors reached a median size of approximately 100 mm³. All test agents (single Abs or combinations), formulated in PBS, were administered IP on Days 6, 10 and 14 at 200 μg per dose in a volume of 200 μl. Tumor volumes, body weights and clinical observations were noted to establish efficacy and tolerability of test agents. Tumor caliper measurements were converted into tumor volumes using the formula: volume=½ (length×width×height). Tumor growth and body weight were monitored for up to 85 days post-implantation. Mice that remained tumor free for at least 45 days from the first zero tumor measurement were deemed to be officially “cured”.

On study, mice received sterile rodent chow and water ad libitum and were housed in sterile filter-top cages with 12-h light/dark cycles. All experiments were conducted in accordance with the guidelines of the Association for Assessment and Accreditation of Laboratory Animal Care International.

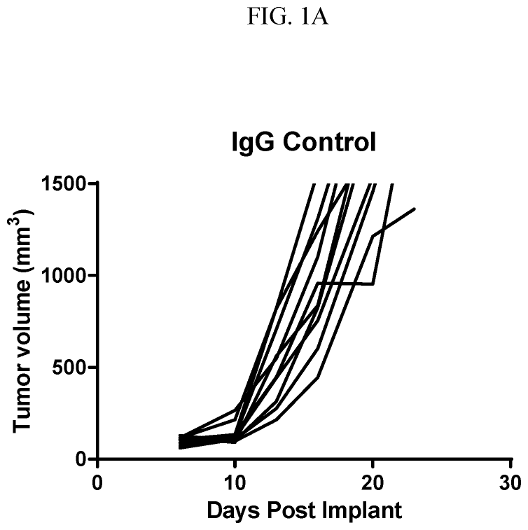

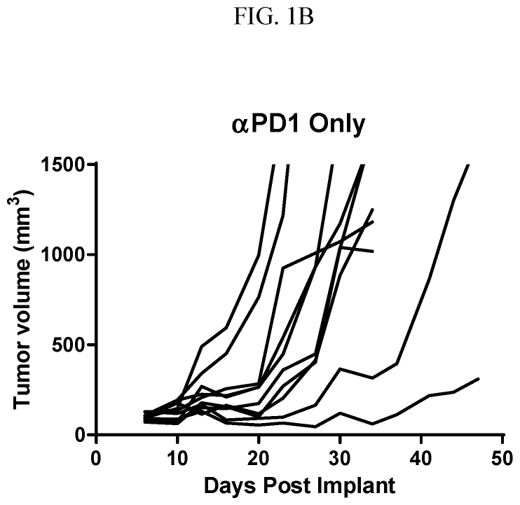

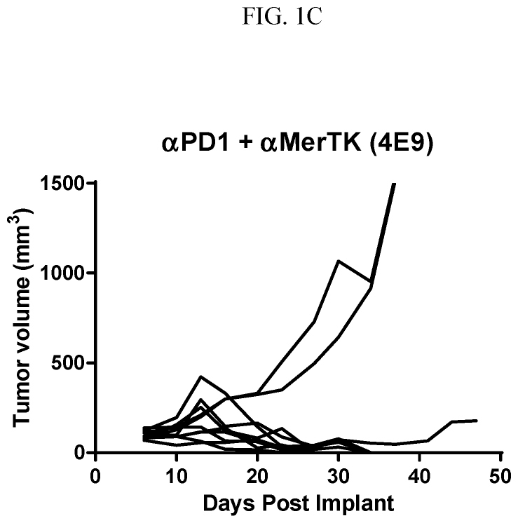

FIG. 1B shows that treatment of mice with the anti-PD-1 Ab significantly reduced the rate of tumor growth compared to the rate of growth of tumors treated with a control mouse IgG1 mAb (human anti-diphtheria toxin (DT) mAb with a mouse IgG1 isotype; simply designated “IgG1”) control, but did not completely shrink the tumor in any mice by Day 47 (FIG. 1A). Treatment of mice with a combination of the anti-PD-1 and anti-MerTK 4E9-IgG1 mAbs further markedly reduced the rate of tumor growth, with 7 out of the 10 mice being effectively cured of the tumor by Day 34 post-implantation (FIG. 1C). Thus, the combination of anti-PD-1 and anti-MerTK shows a strong synergistic effect in inhibiting growth of MC38 colon adenocarcinomas. A combination of Abs is considered synergistic if the antitumor effect of the combination is greater than the sum of the level of inhibition exhibited by each Ab individually.

### Example 5

### Mice Cured Mice from Treatment with Combination of Anti-MERTK and Anti-PD-1 are Resistant to Tumor Growth Upon Rechallenge

In this experiment, the 7 C57BL/6 mice cured of the MC38 tumors by treatment with the combination of the anti-PD-1 and anti-MerTK Abs (Example 4) were rechallenged by SC injection with 10⁶ MC38 tumor cells. A control group of 10 C57BL/6 mice were each injected SC with 10⁶ MC38 tumor cells, and tumor growth in both groups of mice was monitored for at least 23 days post-implantation.

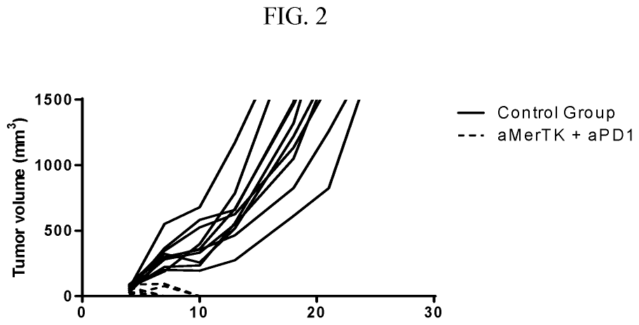

The tumors in the control group grew rapidly, reaching a volume of 1,500 mm³ by 15-23 days post-implantation. In contrast, all 7 of the cured mice were completely resistant to MC38 tumor growth (FIG. 2).

### Example 6

### Two Different Anti-MERTK Abs Comprising Different Fc Regions Exhibit Similar Anti-Tumor Activity and Similar Enhancement of Anti-PD-1 Efficacy

The anti-tumor activity of the mouse anti-MerTK Abs, 2D9 and 4E9, was assessed as monotherapy or in combination with the anti-PD-1 Ab, 4H2, in the MC38 tumor model. Two isotypes of the MerTK Abs were used, the IgG1 isotype and an IgG1-D265A isotype which is a non-FcγR-binding mutant (Clynes et al., 2000). This IgG1-D265A isotype has been shown to reduce the anti-tumor activity of anti-CTLA-4 and anti-GITR Abs in the MC38 tumor model compared to the mouse IgG2a and IgG1 isotype, in contrast to the anti-PD-1 IgG2a isotype exhibiting lower anti-tumor activity than the anti-IgG1 or IgG1-D265A isotypes (WO 2014/089113).

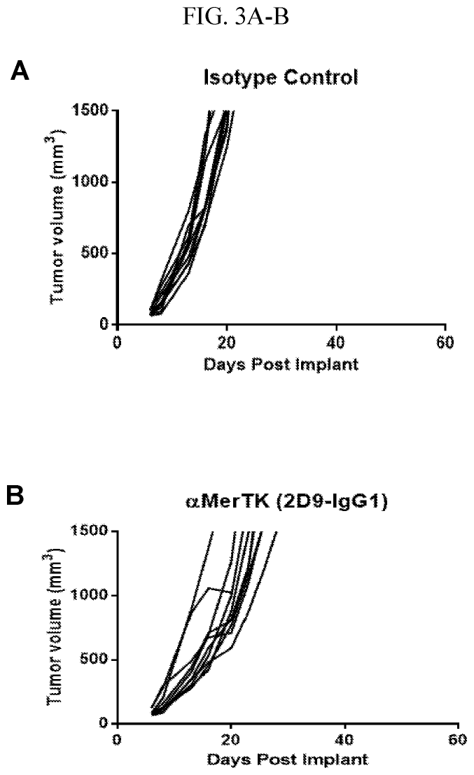

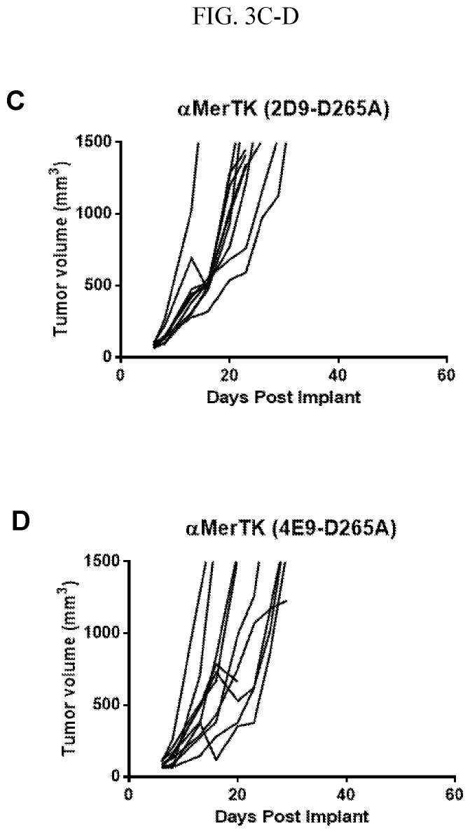

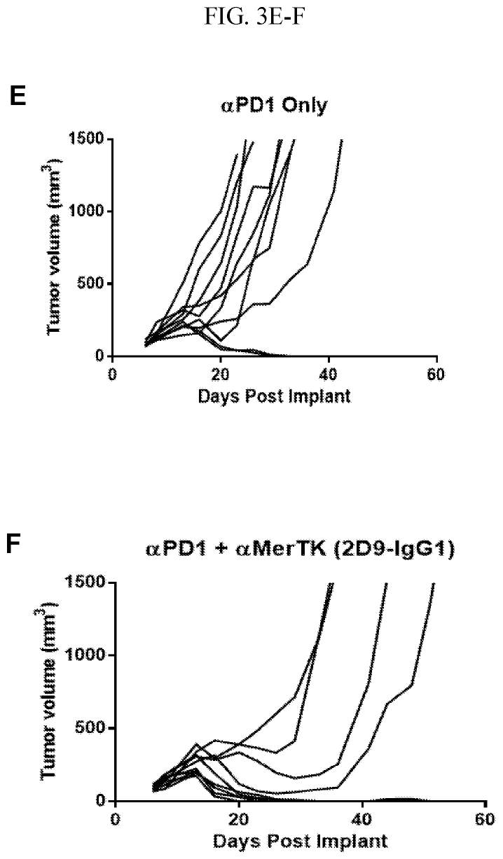

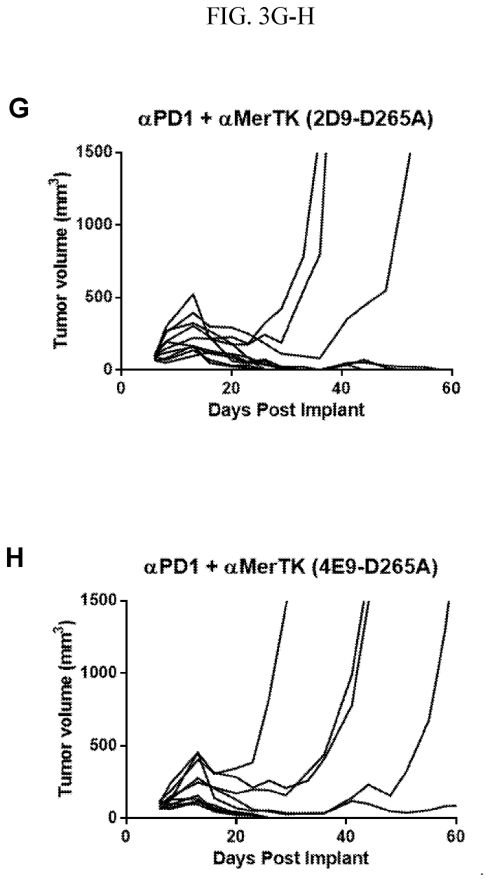

C57BL/6 mice were each injected SC with 10⁶ MC38 tumor cells and randomized into treatment groups (10 mice/group) as previously described (Example 4). The test agents comprised a mouse IgG1 control, the IgG1 and IgG1-D265A isotypes of anti-MerTK mAb 2D9, the IgG1-D265A isotype of anti-MerTK mAb 4E9, anti-PD-1 mAb 4H2, and combinations of the anti-MerTK and anti-PD-1 Abs as indicated in FIG. 3.

The 2D9-IgG1Ab (FIG. 3B) caused a slight inhibition of tumor growth compared to the IgG1 control (FIG. 3A). The 2D9-D265A isotype (FIG. 3C) caused a generally similar, or marginally higher, level of tumor growth inhibition than the IgG1 isotype. A similar level of tumor growth inhibition was induced by the 2D9-D265A and 4E9-D265A Abs (FIGS. 3C and D).

The anti-PD-1 produced significant tumor growth inhibition, with complete tumor rejection in 2 of the 10 mice treated (FIG. 3E).

The combination of the anti-PD-1 and anti-MerTK 2D9-IgG1 Abs resulted in even greater inhibition of tumor growth, with complete tumor rejection in 5 of 9 treated mice (FIG. 3F). Combinations of the anti-PD-1 and anti-MerTK 2D9-D265A or 4E9-D265A Abs produced similar synergistic levels of tumor growth inhibition, with complete tumor rejection in 7 of 9 and 5 of 10 treated mice, respectively (FIGS. 3G and H). Thus, a similar synergistic level of tumor growth-inhibiting efficacy was observed with the two different mouse anti-MerTK Abs (4E9 and 2D9) administered, and similar efficacy was observed irrespective of Fc receptor (FcR) effector function, i.e., IgG1 isotype compared to IgG1-D265A.

The enhanced efficacy of the combination of anti-PD-1 and anti-MerTK Abs in inhibiting tumor growth in the MC38 model compared to anti-PD-1 monotherapy was reproducible across a range of anti-MerTK Ab doses. When administered as monotherapy, anti-MerTK 4E9 at a dose of 1 mg/kg body weight exhibited little effect in inhibiting tumor growth but showed moderate inhibition of tumor growth at 1 mg/kg, albeit much less that the tumor growth inhibition observed with anti-PD-1 (data not shown). The combination of anti-PD-1 with anti-MerTK 4E9-IgG1 at 1 or 3 mg/kg both drastically inhibited tumor growth, with 7 out of 11 and 9 out of 11 mice, respectively, showing complete tumor rejection (data not shown). The combination of anti-PD-1 with 10 mg/kg of anti-MerTK 4E9-IgG1 drastically inhibited tumor growth in practically all of the mice, but the cure rate remained unchanged with 8 out of 11 mice showing complete tumor rejection (data not shown).

### Example 7

### Anti-MERTK Enhances Anti-Tumor Activity of Anti-PD-1 in CT26 Tumor Model

The anti-tumor activity of the mAb 4E9 was also assessed as monotherapy and in in combination with an anti-PD-1 Ab in the CT26 colon adenocarcinoma mouse tumor model.

BALB/c mice were each injected SC with 10⁶ CT26 tumor cells. Mice were randomized into treatment groups of 10 mice/group after 6 days when tumors reached a median size of approximately 100 mm³, and Abs (single Abs or combinations) were administered IP on Days 6, 10 and 14 at 200 μg per dose in a volume of 200 μl. Tumor volumes were measured twice weekly for up to 85 days post-implantation to establich official cures.

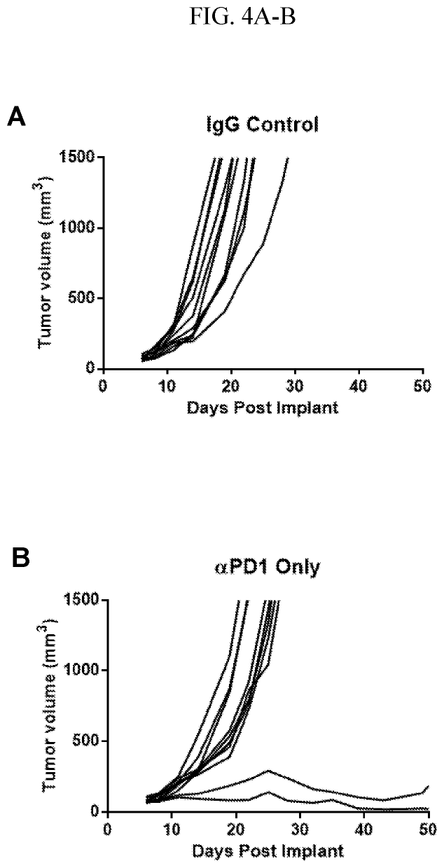

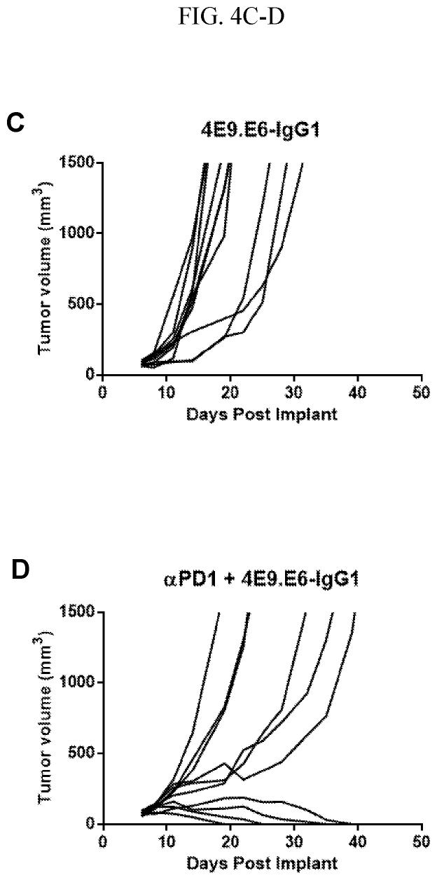

As shown in FIG. 4B, treatment with anti-PD-1 Ab had a moderate effect on reducing the rate of tumor growth in the majority of mice compared to the rate of growth of tumors treated with a mouse IgG1 control (FIG. 4A), but tumor growth was significantly inhibited in one mouse, and one other mouse showed complete tumor rejection. The 4E9-IgG1 Ab showed slight activity in inhibiting tumor growth compared to the IgG1 control (FIG. 4C), whereas treatment with a combination of the anti-PD-1 and anti-MerTK 4E9-IgG1 Abs potently reduced the rate of tumor growth, with 4 out of the 10 mice being cured of the tumor by Day 38 post-implantation (FIG. 4D). Thus, anti-PD-1 and anti-MerTK Abs also interact synergistically to inhibit growth of CT26 colon adenocarcinomas. Overall, the pattern of response of the CT26 tumors to treatment with anti-PD-1, anti-MerTK or a combination of both Abs (FIG. 4) was similar to that seen in the MC38 tumor model (FIGS. 1 and 3), but with growth inhibition being generally somewhat more pronounced in the MC38 model.

### Example 8

### Anti-MERTK MAB, 16B9, Enhances Anti-Tumor Activity of Anti-PD-1 in MC38 Tumor Model

As shown in Examples 4 and 6, both anti-MerTK mAbs 2D9 and 4E9 combine synergistically with anti-PD-1 to potently inhibit growth of MC38 colon adenocarcinomas. As described in Example 3, mAbs 2D9 and 4E9 are similar to the extent they both bind with high affinity to mouse, human and cynomolgus monkey MerTK and are assigned to Bin 2 on hMerTK. A third anti-MerTK moMAb, 16B9, differs from 2D9 and 4E9 in that it binds with high affinity to mMerTK but not to human or cynomolgus monkey MerTK. As it does not bind to hMerTK, it could not be assigned to any hMerTK bin, but this lack of binding to hMerTK suggests that mAb16B9 binds to a different epitope than the epitope bound by 2D9 or 4 E9.

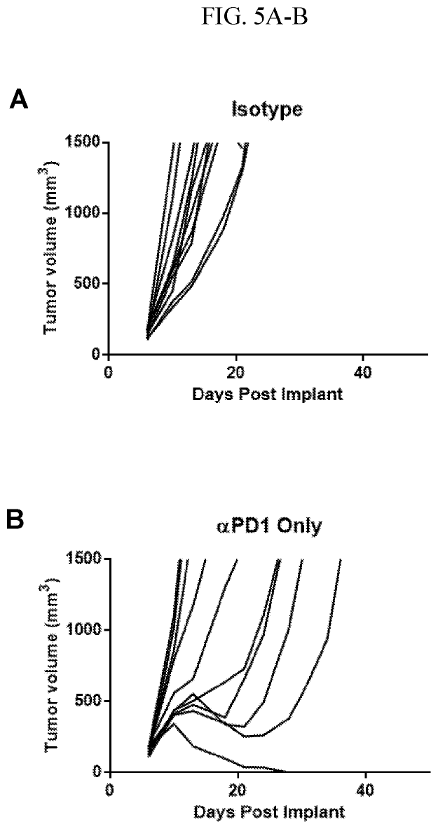

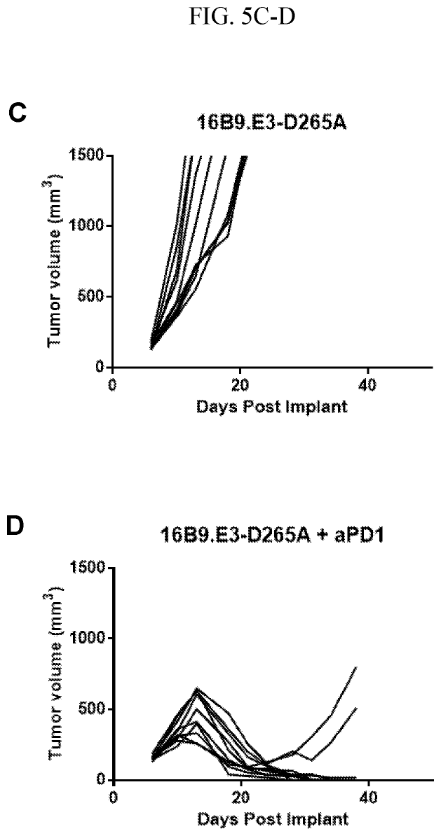

The anti-tumor activity of anti-MerTK mAb, 16B9-D265A, was assessed, either alone or in combination with anti-PD-1 mAb, 4H2, in the MC38 tumor model. The Abs were administered to groups of 10 C57BL/6 mice implanted with MC38 tumors as described in Example 4. As previously demonstrated in Examples 4 and 6, anti-PD1 treatment significantly inhibited MC38 tumor growth (FIG. 5B) compared to the anti-DT IgG1 control (“Isotype”; FIG. 5A), with 1 out of 10 anti-PD-1-treated mice showing complete tumor rejection. In contrast, no single-agent activity in inhibiting tumor growth was seen with the 16B9-D265A anti-MerTK Ab, which produced results comparable to the IgG1 control. Notwithstanding this absence of tumor growth inhibition by 16B9-D265A, the combination of this Ab and anti-PD-1 produced a strong synergistic interaction, evidenced by a massive enhancement of the anti-tumor activity observed with anti-PD-1, including complete tumor rejection in 7 out of 10 mice (FIG. 5D).

### REFERENCES

Abhinandan K R, Martin A C (2008) Analysis and improvements to Kabat and structurally correct numbering of antibody variable domains. Mol Immunol 45:3832-3839.
Akalu Y T, Rothlin C V, Ghosh S (2017) TAM receptor tyrosine kinases as emerging targets of innate immune checkpoint blockade for cancer therapy. Immunol Rev 276(1):165-77.
Al-Lazikani, Lesk A M, Chothia C (1997) Standard conformations for the canonical structures of immunoglobulins. J Mol Biol 273(4):927-48.
Baitsch L, Legat A, Barba L, Fuertes Marraco S A, Rivals J P et al. (2012) Extended co-expression of inhibitory receptors by human CD8 T-cells depending on differentiation, antigen-specificity and anatomical localization. PloS One 7(2): e30852.
Barker R N, Erwig L P, Hill K S, Devine A, Pearce W P et al. (2002) Antigen presentation by macrophages is enhanced by the uptake of necrotic, but not apoptotic, cells. Clin Exp Immunol 127(2): 220-5.
Bondanza A, Zimmermann V S, Rovere-Querini P, Turnay J, Dumitriu I E et al. (2004) Inhibition of phosphatidylserine recognition heightens the immunogenicity of irradiated lymphoma cells in vivo. J Exp Med 200(9):1157-1165.
Brahmer J R, Drake C G, Wollner I, Powderly J D, Picus J et al. (2010) Phase I study of single-agent anti-programmed death-1 (MDX-1106) in refractory solid tumors: safety, clinical activity, pharmacodynamics, and immunologic correlates. J Clin Oncol 28:3167-75.
Brahmer J R, Hammers H, Lipson E J (2015) Nivolumab: targeting PD-1 to bolster antitumor immunity. Future Oncol 11(9):1307-26.
Brahmer J R, Tykodi S S, Chow L Q, Hwu W J, Topalian S L et al. (2012) Safety and activity of anti-PD-L1 antibody in patients with advanced cancer. N Engl J Med 366:2455-65.
Callahan M, Postow M A, Wolchok J D (2016) Targeting T cell co-receptors for cancer therapy. Immunity 44(5):1069-78.
Caberoy N B, Alvarado G, Bigcas J L and Li W (2012) Galectin-3 is a new MerTK-specific eat-me signal. J Cell Physiol 227(2): 401-7.
Caberoy N B, Zhou Y, Li W (2010) Tubby and tubby-like protein 1 are new MerTK ligands for phagocytosis. EMBO J29(23):3898-910.
Callahan M K, Postow M A, Wolchok J D (2016) Targeting T Cell Co-receptors for Cancer Therapy. Immunity 44(5):1069-78.
Chakravarthi BVSK, Nepal S, Varambally S (2016) Genomic and epigenomic alterations in cancer. Am J Pathol 186(7): 1724-35.
Chen D S, Mellman I (2013) Oncology meets immunology: the cancer-immunity cycle. Immunity 39(1), 1-10.
Chothia C, Lesk A M (1987) Canonical structures for the hypervariable regions of immunoglobulins. J Mol Biol 196:901-17.
Chothia C, Lesk A M, Tramontano A, Levitt M, Smith-Gill J S et al. (1989) Conformations of immunoglobulin hypervariable regions. Nature 342:877-83.
Clynes R A, Towers T L, Presta L G, Ravetch J V et al. (2000) Inhibitory Fc receptors modulate in vivo cytotoxicity against tumor targets. Nat Med 6:443-46.
Cook R S, Jacobsen K M, Wofford A M, DeRyckere D, Stanford J et al. (2013) MerTK inhibition in tumor leukocytes decreases tumor growth and metastasis. J Clin Invest 123(8): 3231-42.
Drugs.com—Opdivo Approval History: https://www.drugs.com/history/opdivo.html, last accessed Oct. 8, 2018.
Farkona et al. (2016) Cancer immunotherapy: the beginning of the end of cancer? BMC Medicine 14:73.
Gorelik L, Avgerinos G, Kunes Y, Marasco W A (2017) Preclinical characterization of a novel fully human IgG1 anti-PD-L1 mAb CK-301. In: Proceedings of the American Association for Cancer Research (AACR) Annual Meeting, Apr. 1-5, 2017, Cancer Res 77(13 Suppl): Abstract No. 4606.
Graham D K, DeRyckere D, Davies K D, Earp H S (2014) The TAM family: phosphatidylserine sensing receptor tyrosine kinases gone awry in cancer. Nature Rev Cancer 14: 769-85.
Guo L, Zhang H, Chen B (2017) Nivolumab as Programmed Death-1 (PD-1) inhibitor for targeted immunotherapy in tumor. J Cancer 8(3):410-416.
Herbst R S, Soria J C, Kowanetz M, Fine G D, Hamid 0 et al. (2014) Predictive correlates of response to the anti-PD-L1 antibody MPDL3280A in cancer patients. Nature 515: 563-7.
Hollinger and Hudson (2005) Engineered antibody fragments and the rise of single domains. Nature Biotech 23(9):1126-36.
Iwai Y, Hamanishi J, Chamoto K, Honjo T (2017) Cancer immunotherapies targeting the PD-1 signaling pathway. J Biomed Sci 24(1):26.
Jinushi M, Yagita H, Yoshiyama H, Tahara H (2013) Putting the brakes on anticancer therapies: suppression of innate immune pathways by tumor-associated myeloid cells. Trends Mol Med 19(9): 536-45.
Kabat E A, Wu T T, Bilofsky H, Reid-Miller M, Perry H (1983) Sequence of proteins of immunological interest. Bethesda: National Institute of Health; 1983. 323
Kamta J, Chaar M, Ande A, Altomare D A, Ait-Oudhia S (2017) Advancing cancer therapy with present and emerging immuno-oncology approaches. Front Oncol 18(7):64.
Kaufman R J, Sharp P A (1982) Amplification and expression of sequences cotransfected with a modular dihydrofolate reductase complementary DNA gene. Mol Biol 159:601-21.
Lee-Sherick A B, Eisenman K M, Sather S, McGranahan A et al. (2013) Aberrant MER receptor tyrosine kinase expression contributes to leukemogenesis in acute myeloid leukemia. Oncogene 32: 5359-68.
Lefranc M P, Pommie C, Ruiz M, Giudicelli V, Foulquier E et al. (2003) IMGT unique numbering for immunoglobulin and T cell receptor variable domains and Ig superfamily V-like domains. Dev Comp Immunol 27:55-77.
Lesokhin A M, Callahan M K, Postow M A, Wolchok J D (2015) On being less tolerant: enhanced cancer immunosurveillance enabled by targeting checkpoints and agonists of T cell activation. Sci Transl Med 7(280):280sr1.
Linger R M, Cohen R A, Cummings C T, Sather S et al. (2013) MER or AXL receptor tyrosine kinase inhibition promotes apoptosis, blocks growth and enhances chemosensitivity of human non-small cell lung cancer. Oncogene 32: 3420-3431.
Linger R M, Keating A K, Earp H S, Graham D K (2008) TAM receptor tyrosine kinases: biologic functions, signaling, and potential therapeutic targeting in human cancer. Adv Cancer Res 100: 35-83.
Lipson E J, Sharfman W H, Drake C G, Wollner I, Taube J M et al. (2013) Durable cancer regression off-treatment and effective reinduction therapy with an anti-PD-1 antibody. Clin Cancer Res 19:462-8.
Liu S Y, Wu Y L (2017) Ongoing clinical trials of PD-1 and PD-L1 inhibitors for lung cancer in China. J Hematol Oncol 10(1):136.
Lonberg, N (1994) Transgenic approaches to human monoclonal antibodies. Handbook of Experimental Pharmacology 113:49-101.
Lonberg N, Taylor L D, Harding F A, Trounstine M, Higgins K M et al. (1994) Antigen-specific human antibodies from mice comprising four distinct genetic modifications. Nature 368(6474): 856-9.
Mahoney K M, Rennert P D, Freeman G J (2015) Combination cancer immunotherapy and new immunomodulatory targets. Nat Rev Drug Discov 14(8):561-84.
Martin A, Cheetham J C, Rees A R (1989) Modeling antibody hypervariable loops: a combined algorithm. Proc Natl Acad Sci USA 86(23):9268-72.
MacCallum R M., Martin A C R, Thornton J T (1996) Antibody-antigen interactions: contact analysis and binding site topography. J Mol Biol 262:732-745.
Mellman I, Coukos G, Dranoff (2011) Cancer immunotherapy comes of age. Nature 480: 480-9.
Nguyen K Q, Tsou W I, Calarese D A, Kimani S G, Singh S et al. (2014). Overexpression of MERTK receptor tyrosine kinase in epithelial cancer cells drives efferocytosis in a gain-of-function capacity. J Biol Chem 289(37): 25737-49.
Olafsen and Wu (2010) Antibody vectors for imaging. Semin Nucl Med 40(3):167-81.
Ott P A, Hodi F S, Kaufman H L, Wigginton J M, Wolchok J D (2017) Combination immunotherapy: a road map. J Immunother Cancer 5:16.
Pardoll D M (2012) The blockage of immune checkpoints in cancer immunotherapy. Nat Rev Cancer 12: 252-64.
PCT Publication No. WO 2006/121168, published Nov. 16, 2006 by ONO Pharmaceutical Co., Ltd. and Medarex, Inc.
PCT Publication No. WO 2008/156712, published Dec. 24, 2008 by Organon N V.
PCT Publication No. WO 2012/145493, published Oct. 26, 2012 by Amplimmune, Inc.
PCT Publication No. WO 2013/173223, published Nov. 21, 2013 by Bristol-Myers Squibb Co.
PCT Publication No. WO 2014/089113, published Jun. 12, 2014 by Bristol-Myers Squibb Co.
PCT Publication No. WO 2014/179664, published Nov. 6, 2014 by AnaptysBio, Inc.
PCT Publication No. WO 2014/194302, published Dec. 4, 2014 by Sorrento Therapeutics, Inc.
PCT Publication No. WO 2014/206107, published Dec. 31, 2014 by Shanghai Junshi Biosciences Inc.
PCT Publication No. WO 2015/035606, published Mar. 19, 2015 by Beigene, Ltd.
PCT Publication No. WO 2015/085847, published Jun. 18, 2015 by Shanghai Hengrui Pharmaceutical Co., Ltd.
PCT Publication No. WO 2015/112800, published Jul. 30, 2015 by Regeneron Pharmaceuticals, Inc.
PCT Publication No. WO 2015/112900, published Jul. 30, 2015 by Dana-Farber Cancer Institute, Inc. and Novartis A G
PCT Publication No. WO 2016/106159, published Jun. 30, 2016 by Enumeral Biomedical Holdings, Inc.
PCT Publication No. WO 2016/106221, published Jun. 30, 2016 by The Rockefeller University.
PCT Publication No. WO 2016/149201, published Sep. 22, 2016 by Cytomx Therapeutics, Inc.
PCT Publication No. WO 2016/197367, published Dec. 15, 2016 by Wuxi Biologics (Shanghai) Co. Ltd.
PCT Publication No. WO 2017/020291, published Feb. 9, 2017 by Wuxi Biologics (Shanghai) Co. Ltd.
PCT Publication No. WO 2017/020858, published Feb. 9, 2017 by Wuxi Biologics (Shanghai) Co. Ltd.
PCT Publication No. WO 2017/024465, published by Feb. 16, 2017 Innovent Biologics (Suzhou) Co., Ltd.
PCT Publication No. WO 2017/024515, published Feb. 16, 2017 by Wuxi Biologics (Cayman) Inc.
PCT Publication No. WO 2017/025016, published Feb. 16, 2017 by Innovent Biologics (Suzhou) Co., Ltd.
PCT Publication No. WO 2017/025051, published Feb. 16, 2017 by Wuxi Biologics (Cayman) Inc.
PCT Publication No. WO 2017/034916, published Mar. 2, 2017 by Eli Lilly and Co.
PCT Publication No. WO 2017/040790, published Mar. 9, 2017 by Agenus Inc.
PCT Publication No. WO 2017/106061, published Jun. 22, 2017 by Macrogenics, Inc.
PCT Publication No. WO 2017/123557, published Jul. 20, 2017 by Armo Biosciences, Inc.
PCT Publication No. WO 2017/132827, published Aug. 10, 2017 by Innovent Biologics (Suzhou) Co., Ltd.
PCT Publication No. WO 2017/133540, published Aug. 10, 2017 by Innovent Biologics (Suzhou) Co., Ltd.
PCT Publication No. WO 2019/005756, published Jan. 3, 2019 by The Rockefeller University and Rgenix, Inc.
Pianko M J, Liu Y, Bagchi S, Lesokhin A M (2017) Immune checkpoint blockade for hematologic malignancies: a review. Stem Cell Investig 4:32.
Stitt T N, Conn G, Gore M, Lai C, Bruno J et al. (1995) The anticoagulation factor protein-S and its relative, Gas6, are ligands for the Tyro 3/Axl family of receptor tyrosine kinases. Cell 80:661-70.
Tsou W I, Nguyen K Q, Calarese D A, Garforth S J, Antes A L et al. (2014) Receptor tyrosine kinases, TYRO3, AXL, and MER, demonstrate distinct patterns and complex regulation of ligand-induced activation. J Biol Chem 289(37):25750-63.
U.S. Pat. No. 6,808,710, issued Oct. 26, 2004 to Wood et al.
U.S. Pat. No. 7,488,802, issued Feb. 10, 2009 to Collins et al.
U.S. Pat. No. 7,943,743, issued May 17, 2011 to Korman et al.
U.S. Pat. No. 7,767,429, issued Aug. 3, 2010 to Bookbinder et al.
U.S. Pat. No. 8,008,449, issued Aug. 30, 2011 to Korman et al.
U.S. Pat. No. 8,168,757, issued May 1, 2012 to Finnefrock et al.
U.S. Pat. No. 8,217,149, issued Jul. 10, 2012 to Irving et al.
U.S. Pat. No. 8,354,509, issued Jan. 15, 2013 to Carven et al.
U.S. Pat. No. 8,779,108, issued Jul. 15, 2014 to Queva et al.
U.S. Pat. No. 9,175,082, issued Nov. 3, 2015 to Zhou et al.
U.S. Pat. No. 9,205,148, issued Dec. 3, 2015 to Langermann et al.
U.S. Pat. No. 9,624,298, issued Apr. 18, 2017 to Nastri et al.
U.S. Publication No. 2015/0079109, published Mar. 19, 2015 by Li et al.
U.S. Publication No. 2016/0272708, published Sep. 22, 2016 by Chen et al.
Wang C, Thudium K B, Han M, Wang X T et al. (2014) In vitro characterization of the anti-PD-1 antibody nivolumab, BMS-936558, and in vivo toxicology in non-human primates. Cancer Imm Res 2(9):846-56.
Weber J (2010) Immune checkpoint proteins: a new therapeutic paradigm for cancer-preclinical background: CTLA-4 and PD-1 blockade. Semin Oncol 37(5): 430-9.
Wolchok J D, Weber J S, Maio M, Neyns B, Harmankaya K et al. (2013) Four-year survival rates for patients with metastatic melanoma who received ipilimumab in phase II clinical trials. Ann Oncol 24(8):2174-80.
Wu T T, Kabat E A (1970) An analysis of the sequences of the variable regions of Bence Jones proteins and myeloma light chains and their implications for antibody complementarity. J Exp Med 132:211-250.
Yao S, Zhu Y, Chen L (2013) Advances in targeting cell surface signalling molecules for immune modulation. Nature Rev Drug Discov 12:130-46.
Zhang F, Wei H, Wang X, Bai Y, Wang P et al. (2017) Structural basis of a novel PD-L1 nanobody for immune checkpoint blockade. Cell Discov 3:17004.
Zizzo G, Hilliard B A, Monestier M, Cohen P L (2012) Efficient clearance of early apoptotic cells by human macrophages requires M2c polarization and MerTK induction. J Immunol 189(7):3508-20.

1. A monoclonal antibody, or an antigen-binding portion thereof, that specifically binds to proto-oncogene tyrosine-protein kinase MER (MerTK) expressed on the surface of a cell and inhibits efferocytosis by the MerTK-expressing cell.

2. The monoclonal antibody or antigen-binding portion thereof of claim 1, which inhibits efferocytosis of the human MerTK (hMerTK)-expressing cell with an IC₅₀ of:
(a) about 5 nM or lower;
(b) about 1 nM or lower;
(c) about 0.1 nM or lower;
(d) between about 0.01 nM and about 1 nM;
(e) between about 0.01 nM and about 0.7 nM;
(f) between about 0.04 nM and about 0.7 nM; or
(g) between about 0.04 nM and about 0.1 nM.

3. The monoclonal antibody or antigen-binding portion thereof of claim 1, which inhibits binding of growth arrest-specific protein 6 (Gas6) to hMerTK and inhibits MerTK/Gas6 signaling.

4. The monoclonal antibody or antigen-binding portion thereof of claim 3, which inhibits MerTK/Gas6 signaling with an IC₅₀ of:
(a) about 50 nM or lower;
(b) about 10 nM or lower;
(c) about 5 nM or lower;
(d) about 1 nM or lower;
(e) about 0.5 nM or lower;
(f) about 0.1 nM or lower;
(g) between about 0.01 nM and about 10 nM;
(h) between about 0.05 nM and about 6 nM;
(i) between about 0.08 nM and about 2 nM; or
(j) between about 0.2 nM and about 2 nM.

5. The monoclonal antibody or antigen-binding portion thereof of claim 1, which specifically binds to human MerTK, the sequence of which is set forth as SEQ ID NO: 259.

6. The monoclonal antibody or antigen-binding portion thereof of claim 5, which binds to human MerTK with a K_{D} of:
(a) about 100 nM or lower;
(b) about 50 nM or lower;
(c) about 10 nM or lower;
(d) about 5 nM or lower;
(e) about 1 nM or lower;
(f) about 0.5 nM or lower;
(g) about 0.1 nM or lower;
(h) about 0.05 nM or lower;
(i) about 0.01 nM or lower;
(j) between about 100 nM and about 0.1 nM;
(k) between about 50 nM and about 0.5 nM;
(l) between about 10 nM and about 1 nM; or
(m) between about 6 nM and about 2 nM.

7. The monoclonal antibody or antigen-binding portion thereof of claim 1, which specifically binds to cynomolgus monkey MerTK, the sequence of which is set forth as SEQ ID NO: 260.

8. The monoclonal antibody or antigen-binding portion thereof of claim 7, which binds to cynomolgus monkey MerTK with a K_{D} of:
(a) about 100 nM or lower;
(b) about 50 nM or lower;
(c) about 10 nM or lower;
(d) about 5 nM or lower;
(e) about 1 nM or lower;
(f) about 0.5 nM or lower;
(g) about 0.1 nM or lower;
(h) between about 100 nM and about 0.1 nM;
(i) between about 50 nM and about 0.5 nM;
(j) between about 10 nM and about 1 nM; or
(k) between about 5 nM and about 1 nM.

9. The monoclonal antibody or antigen-binding portion thereof of claim 1, which specifically binds to murine MerTK, the sequence of which is set forth as SEQ ID NO: 261.

10. The monoclonal antibody or antigen-binding portion thereof of claim 9, which binds to mouse MerTK with a K_{D} of:
(a) about 100 nM or lower;
(b) about 50 nM or lower;
(c) about 10 nM or lower;
(d) about 5 nM or lower;
(e) about 1 nM or lower;
(f) about 0.5×nM or lower;
(g) about 0.1 nM or lower;
(h) between about 100 nM and about 0.1 nM;
(i) between about 50 nM and about 0.5 nM;
(j) between about 10 nM and about 1 nM; or
(k) between about 5 nM and about 1 nM.

11. The monoclonal antibody or antigen-binding portion thereof of claim 1, which cross-reacts with:
(a) at least both human and cynomolgus monkey MerTK;
(b) at least both human and murine MerTK; or
(c) human, cynomolgus monkey and murine MerTK.

12. A monoclonal antibody, or an antigen-binding portion thereof, which specifically binds to a Bin 1 epitope on human proto-oncogene tyrosine-protein kinase MER (hMerTK), the sequence of which is set forth as SEQ ID NO: 259, wherein the epitope is located in the first Ig domain of hMerTK within a region spanning approximately amino acid residues 105 to 165 as determined by yeast display and/or hydrogen-deuterium exchange mass spectrometry (HDX-MS) epitope mapping.

13. The monoclonal antibody or antigen-binding portion thereof of claim 12, wherein the Bin 1 epitope:
(a) is located in within a region of hMerTK spanning approximately amino acid residues 126 to 155 as determined by HDX-MS epitope mapping; or
(b) comprises at least one, two, three, four, five, six, seven, ten, twenty or all of the amino acid residues 126 to 155 as determined by HDX-MS epitope mapping.

14. A monoclonal antibody, or an antigen-binding portion thereof, which specifically binds to a Bin 2 epitope on human proto-oncogene tyrosine-protein kinase MER (hMerTK), the sequence of which is set forth as SEQ ID NO: 259, wherein the epitope is located in the second Ig domain of hMerTK within a region spanning approximately amino acid residues 195 to 270 as determined by yeast display and/or hydrogen-deuterium exchange mass spectrometry (HDX-MS) epitope mapping.

15. The monoclonal antibody or antigen-binding portion thereof of claim 14, wherein the Bin 2 epitope:
(a) is located in within a region of hMerTK spanning approximately amino acid residues 231 to 249 as determined by HDX-MS epitope mapping;
(b) comprises one, two, three, four, five, six or all of the amino acid residues N234, S236, R237, E240, Q241, P242 and G269 as determined by yeast display epitope mapping;
(c) comprises the amino acid residues N234, S236, R237, E240, Q241, P242 and G269 as determined by yeast display epitope mapping; or
(d) comprises at least one, two, three, four, five, six, seven, ten or all of the amino acid residues 231 to 249 and amino acid residue G269 as determined by HDX-MS and yeast display epitope mapping.

16. A monoclonal antibody, or an antigen-binding portion thereof, which specifically binds to a Bin 3 epitope on human proto-oncogene tyrosine-protein kinase MER (hMerTK), the sequence of which is set forth as SEQ ID NO:259, wherein the epitope is located in the fibronectin (Fn) domains of hMerTK within a region spanning approximately amino acid residues 420 to 490 as determined by yeast display and/or hydrogen-deuterium exchange mass spectrometry (HDX-MS) epitope mapping.

17. A monoclonal antibody, or an antigen-binding portion thereof, which specifically binds to human proto-oncogene tyrosine-protein kinase MER (hMerTK) expressed on the surface of a cell, and comprises the CDR1, CDR2 and CDR3 domains in each of:
(a) a V_{H} comprising consecutively linked amino acids having the sequence set forth as SEQ ID NO: 217 and a V_{L} comprising consecutively linked amino acids having the sequence set forth as SEQ ID NO: 218;
(b) a V_{H} comprising consecutively linked amino acids having the sequence set forth as SEQ ID NO: 221 and a V_{L} comprising consecutively linked amino acids having the sequence set forth as SEQ ID NO: 222;
(c) a V_{H} comprising consecutively linked amino acids having the sequence set forth as SEQ ID NO: 225 and a V_{L} comprising consecutively linked amino acids having the sequence set forth as SEQ ID NO: 226;
(d) a V_{H} comprising consecutively linked amino acids having the sequence set forth as SEQ ID NO: 229 and a V_{L} comprising consecutively linked amino acids having the sequence set forth as SEQ ID NO: 230;
(e) a V_{H} comprising consecutively linked amino acids having the sequence set forth as SEQ ID NO: 233 and a V_{L} comprising consecutively linked amino acids having the sequence set forth as SEQ ID NO: 234;
(f) a V_{H} comprising consecutively linked amino acids having the sequence set forth as SEQ ID NO: 237 and a V_{L} comprising consecutively linked amino acids having the sequence set forth as SEQ ID NO: 238;
(g) a V_{H} comprising consecutively linked amino acids having the sequence set forth as SEQ ID NO: 241 and a V_{L} comprising consecutively linked amino acids having the sequence set forth as SEQ ID NO: 242;
(h) a V_{H} comprising consecutively linked amino acids having the sequence set forth as SEQ ID NO: 245 and a V_{L} comprising consecutively linked amino acids having the sequence set forth as SEQ ID NO: 246;
(i) a V_{H} comprising consecutively linked amino acids having the sequence set forth as SEQ ID NO: 249 and a V_{L} comprising consecutively linked amino acids having the sequence set forth as SEQ ID NO: 250;
(j) a V_{H} comprising consecutively linked amino acids having the sequence set forth as SEQ ID NO: 253 and a V_{L} comprising consecutively linked amino acids having the sequence set forth as SEQ ID NO: 254;
(k) a V_{H} comprising consecutively linked amino acids having the sequence set forth as SEQ ID NO: 255 and a V_{L} comprising consecutively linked amino acids having the sequence set forth as SEQ ID NO: 256; or
(l) a V_{H} comprising consecutively linked amino acids having the sequence set forth as SEQ ID NO: 257 and a V_{L} comprising consecutively linked amino acids having the sequence set forth as SEQ ID NO: 258.

18. The monoclonal antibody of claim 17, which comprises the following CDR domains as defined by the Kabat method:
(a) a heavy chain variable region CDR1 comprising consecutively linked amino acids having the sequence set forth as SEQ ID NO:1; a heavy chain variable region CDR2 comprising consecutively linked amino acids having the sequence set forth as SEQ ID NO: 4; a heavy chain variable region CDR3 comprising consecutively linked amino acids having the sequence set forth as SEQ ID NO:7; a light chain variable region CDR1 comprising consecutively linked amino acids having the sequence set forth as SEQ ID NO:10; a light chain variable region CDR2 comprising consecutively linked amino acids having the sequence set forth as SEQ ID NO:13; and a light chain variable region CDR3 comprising consecutively linked amino acids having the sequence set forth as SEQ ID NO:16; or
(b) a heavy chain variable region CDR1 comprising consecutively linked amino acids having the sequence set forth as SEQ ID NO:73; a heavy chain variable region CDR2 comprising consecutively linked amino acids having the sequence set forth as SEQ ID NO: 76; a heavy chain variable region CDR3 comprising consecutively linked amino acids having the sequence set forth as SEQ ID NO:79; a light chain variable region CDR1 comprising consecutively linked amino acids having the sequence set forth as SEQ ID NO:82; a light chain variable region CDR2 comprising consecutively linked amino acids having the sequence set forth as SEQ ID NO:85; and a light chain variable region CDR3 comprising consecutively linked amino acids having the sequence set forth as SEQ ID NO:88.

19. The monoclonal antibody or antigen-binding portion thereof of claim 17, which comprises:
(a) a V_{H} comprising consecutively linked amino acids having the sequence set forth as SEQ ID NO: 217 and a V_{L} comprising consecutively linked amino acids having the sequence set forth as SEQ ID NO: 218;
(b) a V_{H} comprising consecutively linked amino acids having the sequence set forth as SEQ ID NO: 221 and a V_{L} comprising consecutively linked amino acids having the sequence set forth as SEQ ID NO: 222;
(c) a V_{H} comprising consecutively linked amino acids having the sequence set forth as SEQ ID NO: 225 and a V_{L} comprising consecutively linked amino acids having the sequence set forth as SEQ ID NO: 226;
(d) a V_{H} comprising consecutively linked amino acids having the sequence set forth as SEQ ID NO: 229 and a V_{L} comprising consecutively linked amino acids having the sequence set forth as SEQ ID NO: 230;
(e) a V_{H} comprising consecutively linked amino acids having the sequence set forth as SEQ ID NO: 233 and a V_{L} comprising consecutively linked amino acids having the sequence set forth as SEQ ID NO: 234;
(f) a V_{H} comprising consecutively linked amino acids having the sequence set forth as SEQ ID NO: 237 and a V_{L} comprising consecutively linked amino acids having the sequence set forth as SEQ ID NO: 238;
(g) a V_{H} comprising consecutively linked amino acids having the sequence set forth as SEQ ID NO: 241 and a V_{L} comprising consecutively linked amino acids having the sequence set forth as SEQ ID NO: 242;
(h) a V_{H} comprising consecutively linked amino acids having the sequence set forth as SEQ ID NO: 245 and a V_{L} comprising consecutively linked amino acids having the sequence set forth as SEQ ID NO: 246;
(i) a V_{H} comprising consecutively linked amino acids having the sequence set forth as SEQ ID NO: 249 and a V_{L} comprising consecutively linked amino acids having the sequence set forth as SEQ ID NO: 250;
(j) a V_{H} comprising consecutively linked amino acids having the sequence set forth as SEQ ID NO: 253 and a V_{L} comprising consecutively linked amino acids having the sequence set forth as SEQ ID NO: 254;
(k) a V_{H} comprising consecutively linked amino acids having the sequence set forth as SEQ ID NO: 255 and a V_{L} comprising consecutively linked amino acids having the sequence set forth as SEQ ID NO: 256; or
(l) a V_{H} comprising consecutively linked amino acids having the sequence set forth as SEQ ID NO: 257 and a V_{L} comprising consecutively linked amino acids having the sequence set forth as SEQ ID NO: 258.

20. The monoclonal antibody or antigen-binding portion thereof of claim 17, which comprises:
(a) a heavy chain comprising consecutively linked amino acids having the sequence set forth as SEQ ID NO: 219 and a light chain comprising consecutively linked amino acids having the sequence set forth as SEQ ID NO: 220;
(b) a heavy chain comprising consecutively linked amino acids having the sequence set forth as SEQ ID NO: 223 and a light chain comprising consecutively linked amino acids having the sequence set forth as SEQ ID NO: 224;
(c) a heavy chain comprising consecutively linked amino acids having the sequence set forth as SEQ ID NO: 227 and a light chain comprising consecutively linked amino acids having the sequence set forth as SEQ ID NO: 228;
(d) a heavy chain comprising consecutively linked amino acids having the sequence set forth as SEQ ID NO: 231 and a light chain comprising consecutively linked amino acids having the sequence set forth as SEQ ID NO: 232;
(e) a heavy chain comprising consecutively linked amino acids having the sequence set forth as SEQ ID NO: 235 and a light chain comprising consecutively linked amino acids having the sequence set forth as SEQ ID NO: 236;
(f) a heavy chain comprising consecutively linked amino acids having the sequence set forth as SEQ ID NO: 239 and a light chain comprising consecutively linked amino acids having the sequence set forth as SEQ ID NO: 240;
(g) a heavy chain comprising consecutively linked amino acids having the sequence set forth as SEQ ID NO: 243 and a light chain comprising consecutively linked amino acids having the sequence set forth as SEQ ID NO: 244;
(h) a heavy chain comprising consecutively linked amino acids having the sequence set forth as SEQ ID NO: 247 and a light chain comprising consecutively linked amino acids having the sequence set forth as SEQ ID NO: 248; or
(i) a heavy chain comprising consecutively linked amino acids having the sequence set forth as SEQ ID NO: 251 and a light chain comprising consecutively linked amino acids having the sequence set forth as SEQ ID NO: 252.

21. An immunoconjugate comprising the monoclonal antibody or antigen-binding portion thereof of claim 1, linked to a therapeutic agent, optionally wherein the therapeutic agent is a cytotoxin or a radioactive isotope.

22. A bispecific molecule comprising the monoclonal antibody or antigen-binding portion thereof of claim 1, linked to a binding domain that has a different binding specificity than the monoclonal antibody or antigen-binding portion thereof.

23. A composition comprising:
(a) the monoclonal antibody or antigen-binding portion thereof of claim 1
and a pharmaceutically acceptable carrier.

24. A method for treating a subject afflicted with a cancer, comprising administering to the subject a therapeutically effective amount of the monoclonal antibody or antigen-binding portion thereof of claim 1, optionally in combination an additional therapeutic agent for treating a cancer, such that the subject is treated.

25. The method of claim 24, wherein the additional therapeutic agent is:
(a) an antagonistic antibody that binds specifically to Programmed Death-1 (PD-1), Programmed Death Ligand-1 (PD-L1), Cytotoxic T-Lymphocyte Antigen-4 (CTLA-4), Lymphocyte Activation Gene-3 (LAG-3), B and T lymphocyte attenuator (BTLA), T cell Immunoglobulin and Mucin domain-3 (TIM-3), Killer Immunoglobulin-like Receptor (KIR), Killer cell Lectin-like Receptor G1 (KLRG-1), adenosine A2a receptor (A2aR), Natural Killer Cell Receptor 2B4 (CD244), or CD160; or
(b) an agonistic antibody that binds specifically to Inducible T cell Co-Stimulator (ICOS), CD137 (4-1BB), CD134 (OX40), CD27, Glucocorticoid-Induced TNFR-Related protein (GITR), and HerpesVirus Entry Mediator (HVEM)

26. A kit for treating a subject afflicted with a cancer, the kit comprising:
(a) one or more dosages ranging from about 0.1 to about 20 mg/kg body weight of a monoclonal antibody or an antigen-binding portion thereof that binds specifically to MerTK;
(b) optionally one or more dosages ranging from about 200 to about 1600 mg of a monoclonal antibody or an antigen-binding portion thereof that binds specifically to PD-1 or to PD-L1; and
(b) and instructions for using the monoclonal antibody or portion thereof that binds specifically to MerTK, and optionally the antibody or portion thereof that binds specifically to PD-1 or to PD-L1, in the method of claim 24.


SEQUENCE LISTING (261 sequences)

SEQ ID NO: 1 - protein, 6 aa, Homo sapiens
SGNYWG

SEQ ID NO: 2 - protein, 8 aa, Homo sapiens
GYSISSGN

SEQ ID NO: 3 - protein, 9 aa, Homo sapiens
GYSISSGNY

SEQ ID NO: 4 - protein, 16 aa, Homo sapiens
SVDHSGSTYYSPSLKS

SEQ ID NO: 5 - protein, 5 aa, Homo sapiens
DHSGS

SEQ ID NO: 6 - protein, 7 aa, Homo sapiens
VDHSGST

SEQ ID NO: 7 - protein, 13 aa, Homo sapiens
NTMIRGVMDWFDP

SEQ ID NO: 8 - protein, 13 aa, Homo sapiens
NTMIRGVMDWFDP

SEQ ID NO: 9 - protein, 15 aa, Homo sapiens
ARNTMIRGVMDWFDP

SEQ ID NO: 10 - protein, 11 aa, Homo sapiens
RASQGISSALA

SEQ ID NO: 11 - protein, 11 aa, Homo sapiens
RASQGISSALA

SEQ ID NO: 12 - protein, 6 aa, Homo sapiens
QGISSA

SEQ ID NO: 13 - protein, 7 aa, Homo sapiens
DASSLES

SEQ ID NO: 14 - protein, 7 aa, Homo sapiens
DASSLES

SEQ ID NO: 15 - protein, 3 aa, Homo sapiens
DAS

SEQ ID NO: 16 - protein, 8 aa, Homo sapiens
QQFRSYPT

SEQ ID NO: 17 - protein, 8 aa, Homo sapiens
QQFRSYPT

SEQ ID NO: 18 - protein, 8 aa, Homo sapiens
QQFRSYPT

SEQ ID NO: 19 - protein, 6 aa, Homo sapiens
SDYSWG

SEQ ID NO: 20 - protein, 8 aa, Homo sapiens
GYSISSDY

SEQ ID NO: 21 - protein, 9 aa, Homo sapiens
GYSISSDYS

SEQ ID NO: 22 - protein, 16 aa, Homo sapiens
SIYHSGNTYFNPSLKS

SEQ ID NO: 23 - protein, 5 aa, Homo sapiens
YHSGN

SEQ ID NO: 24 - protein, 7 aa, Homo sapiens
IYHSGNT

SEQ ID NO: 25 - protein, 13 aa, Homo sapiens
DKSYYFGPGSMDV

SEQ ID NO: 26 - protein, 13 aa, Homo sapiens
DKSYYFGPGSMDV

SEQ ID NO: 27 - protein, 15 aa, Homo sapiens
ARDKSYYFGPGSMDV

SEQ ID NO: 28 - protein, 11 aa, Homo sapiens
RASQGISSALA

SEQ ID NO: 29 - protein, 11 aa, Homo sapiens
RASQGISSALA

SEQ ID NO: 30 - protein, 6 aa, Homo sapiens
QGISSA

SEQ ID NO: 31 - protein, 7 aa, Homo sapiens
DASSLES

SEQ ID NO: 32 - protein, 7 aa, Homo sapiens
DASSLES

SEQ ID NO: 33 - protein, 3 aa, Homo sapiens
DAS

SEQ ID NO: 34 - protein, 8 aa, Homo sapiens
QQFKSYLT

SEQ ID NO: 35 - protein, 8 aa, Homo sapiens
QQFKSYLT

SEQ ID NO: 36 - protein, 8 aa, Homo sapiens
QQFKSYLT

SEQ ID NO: 37 - protein, 5 aa, Homo sapiens
SYSMN

SEQ ID NO: 38 - protein, 7 aa, Homo sapiens
GFTFSSY

SEQ ID NO: 39 - protein, 8 aa, Homo sapiens
GFTFSSYS

SEQ ID NO: 40 - protein, 17 aa, Homo sapiens
YIISGSDTIFYADSVKG

SEQ ID NO: 41 - protein, 6 aa, Homo sapiens
ISGSDT

SEQ ID NO: 42 - protein, 8 aa, Homo sapiens
IISGSDTI

SEQ ID NO: 43 - protein, 14 aa, Homo sapiens
DETVVRGVINYFDY

SEQ ID NO: 44 - protein, 14 aa, Homo sapiens
DETVVRGVINYFDY

SEQ ID NO: 45 - protein, 16 aa, Homo sapiens
ARDETVVRGVINYFDY

SEQ ID NO: 46 - protein, 11 aa, Homo sapiens
RSSQGISSALA

SEQ ID NO: 47 - protein, 11 aa, Homo sapiens
RSSQGISSALA

SEQ ID NO: 48 - protein, 6 aa, Homo sapiens
QGISSA

SEQ ID NO: 49 - protein, 7 aa, Homo sapiens
DASSLES

SEQ ID NO: 50 - protein, 7 aa, Homo sapiens
DASSLES

SEQ ID NO: 51 - protein, 3 aa, Homo sapiens
DAS

SEQ ID NO: 52 - protein, 8 aa, Homo sapiens
QQFISYPT

SEQ ID NO: 53 - protein, 8 aa, Homo sapiens
QQFISYPT

SEQ ID NO: 54 - protein, 8 aa, Homo sapiens
QQFISYPT

SEQ ID NO: 55 - protein, 5 aa, Homo sapiens
RYYMY

SEQ ID NO: 56 - protein, 7 aa, Homo sapiens
GNTQIRY

SEQ ID NO: 57 - protein, 8 aa, Homo sapiens
GNTQIRYY

SEQ ID NO: 58 - protein, 17 aa, Homo sapiens
ILNPNQDQTTYAQKFQG

SEQ ID NO: 59 - protein, 6 aa, Homo sapiens
NPNQDQ

SEQ ID NO: 60 - protein, 8 aa, Homo sapiens
LNPNQDQT

SEQ ID NO: 61 - protein, 8 aa, Homo sapiens
TYRYYMDV

SEQ ID NO: 62 - protein, 8 aa, Homo sapiens
TYRYYMDV

SEQ ID NO: 63 - protein, 10 aa, Homo sapiens
ATTYRYYMDV

SEQ ID NO: 64 - protein, 12 aa, Homo sapiens
RASQSVRSNYLA

SEQ ID NO: 65 - protein, 12 aa, Homo sapiens
RASQSVRSNYLA

SEQ ID NO: 66 - protein, 7 aa, Homo sapiens
QSVRSNY

SEQ ID NO: 67 - protein, 7 aa, Homo sapiens
GASSRAT

SEQ ID NO: 68 - protein, 7 aa, Homo sapiens
GASSRAT

SEQ ID NO: 69 - protein, 3 aa, Homo sapiens
GAS

SEQ ID NO: 70 - protein, 9 aa, Homo sapiens
QQYGSSPRT

SEQ ID NO: 71 - protein, 9 aa, Homo sapiens
QQYGSSPRT

SEQ ID NO: 72 - protein, 9 aa, Homo sapiens
QQYGSSPRT

SEQ ID NO: 73 - protein, 5 aa, Homo sapiens
RYYMH

SEQ ID NO: 74 - protein, 7 aa, Homo sapiens
GRTFIRY

SEQ ID NO: 75 - protein, 8 aa, Homo sapiens
GRTFIRYY

SEQ ID NO: 76 - protein, 17 aa, Homo sapiens
IIWPNGDQTTYAQKFQG

SEQ ID NO: 77 - protein, 6 aa, Homo sapiens
WPNGDQ

SEQ ID NO: 78 - protein, 8 aa, Homo sapiens
IWPNGDQT

SEQ ID NO: 79 - protein, 8 aa, Homo sapiens
TYKYAMDV

SEQ ID NO: 80 - protein, 8 aa, Homo sapiens
TYKYAMDV

SEQ ID NO: 81 - protein, 10 aa, Homo sapiens
ATTYKYAMDV

SEQ ID NO: 82 - protein, 12 aa, Homo sapiens
RASQSVRSNYLA

SEQ ID NO: 83 - protein, 12 aa, Homo sapiens
RASQSVRSNYLA

SEQ ID NO: 84 - protein, 7 aa, Homo sapiens
QSVRSNY

SEQ ID NO: 85 - protein, 7 aa, Homo sapiens
GASSRAT

SEQ ID NO: 86 - protein, 7 aa, Homo sapiens
GASSRAT

SEQ ID NO: 87 - protein, 3 aa, Homo sapiens
GAS

SEQ ID NO: 88 - protein, 9 aa, Homo sapiens
QQYESPPRT

SEQ ID NO: 89 - protein, 9 aa, Homo sapiens
QQYESPPRT

SEQ ID NO: 90 - protein, 9 aa, Homo sapiens
QQYESPPRT

SEQ ID NO: 91 - protein, 5 aa, Homo sapiens
IYYWS

SEQ ID NO: 92 - protein, 7 aa, Homo sapiens
GGSFSIY

SEQ ID NO: 93 - protein, 8 aa, Homo sapiens
GGSFSIYY

SEQ ID NO: 94 - protein, 16 aa, Homo sapiens
EINDEGNTNYNPSLKS

SEQ ID NO: 95 - protein, 5 aa, Homo sapiens
NDEGN

SEQ ID NO: 96 - protein, 7 aa, Homo sapiens
INDEGNT

SEQ ID NO: 97 - protein, 11 aa, Homo sapiens
GGTGDIHAFDI

SEQ ID NO: 98 - protein, 11 aa, Homo sapiens
GGTGDIHAFDI

SEQ ID NO: 99 - protein, 13 aa, Homo sapiens
ARGGTGDIHAFDI

SEQ ID NO: 100 - protein, 11 aa, Homo sapiens
RASQGISKWLA

SEQ ID NO: 101 - protein, 11 aa, Homo sapiens
RASQGISKWLA

SEQ ID NO: 102 - protein, 6 aa, Homo sapiens
QGISKW

SEQ ID NO: 103 - protein, 7 aa, Homo sapiens
AASSLQS

SEQ ID NO: 104 - protein, 7 aa, Homo sapiens
AASSLQS

SEQ ID NO: 105 - protein, 3 aa, Homo sapiens
AAS

SEQ ID NO: 106 - protein, 9 aa, Homo sapiens
QQYNSYPWT

SEQ ID NO: 107 - protein, 9 aa, Homo sapiens
QQYNSYPWT

SEQ ID NO: 108 - protein, 9 aa, Homo sapiens
QQYNSYPWT

SEQ ID NO: 109 - protein, 5 aa, Homo sapiens
GYYWS

SEQ ID NO: 110 - protein, 7 aa, Homo sapiens
GGSFSGY

SEQ ID NO: 111 - protein, 8 aa, Homo sapiens
GGSFSGYY

SEQ ID NO: 112 - protein, 16 aa, Homo sapiens
EISHSGSTNYNPSLKS

SEQ ID NO: 113 - protein, 5 aa, Homo sapiens
SHSGS

SEQ ID NO: 114 - protein, 7 aa, Homo sapiens
ISHSGST

SEQ ID NO: 115 - protein, 10 aa, Homo sapiens
ALSRYWYFDL

SEQ ID NO: 116 - protein, 10 aa, Homo sapiens
ALSRYWYFDL

SEQ ID NO: 117 - protein, 12 aa, Homo sapiens
ARALSRYWYFDL

SEQ ID NO: 118 - protein, 11 aa, Homo sapiens
RASQSASNYLA

SEQ ID NO: 119 - protein, 11 aa, Homo sapiens
RASQSASNYLA

SEQ ID NO: 120 - protein, 11 aa, Homo sapiens
RASQSASNYLA

SEQ ID NO: 121 - protein, 7 aa, Homo sapiens
DASNRAT

SEQ ID NO: 122 - protein, 7 aa, Homo sapiens
DASNRAT

SEQ ID NO: 123 - protein, 3 aa, Homo sapiens
DAS

SEQ ID NO: 124 - protein, 9 aa, Homo sapiens
YQRSQWPIS

SEQ ID NO: 125 - protein, 9 aa, Homo sapiens
YQRSQWPIS

SEQ ID NO: 126 - protein, 9 aa, Homo sapiens
YQRSQWPIS

SEQ ID NO: 127 - protein, 5 aa, Homo sapiens
SFAIS

SEQ ID NO: 128 - protein, 7 aa, Homo sapiens
GISLSSF

SEQ ID NO: 129 - protein, 8 aa, Homo sapiens
GISLSSFA

SEQ ID NO: 130 - protein, 16 aa, Homo sapiens
VIWTGGGTDYNSALKS

SEQ ID NO: 131 - protein, 5 aa, Homo sapiens
WTGGG

SEQ ID NO: 132 - protein, 7 aa, Homo sapiens
IWTGGGT

SEQ ID NO: 133 - protein, 6 aa, Homo sapiens
HWYLDV

SEQ ID NO: 134 - protein, 6 aa, Homo sapiens
HWYLDV

SEQ ID NO: 135 - protein, 8 aa, Homo sapiens
ASHWYLDV

SEQ ID NO: 136 - protein, 14 aa, Homo sapiens
RSSTGAVSTSNYAN

SEQ ID NO: 137 - protein, 14 aa, Homo sapiens
RSSTGAVSTSNYAN

SEQ ID NO: 138 - protein, 9 aa, Homo sapiens
TGAVSTSNY

SEQ ID NO: 139 - protein, 7 aa, Homo sapiens
GANSRAP

SEQ ID NO: 140 - protein, 7 aa, Homo sapiens
GANSRAP

SEQ ID NO: 141 - protein, 3 aa, Homo sapiens
GAN

SEQ ID NO: 142 - protein, 9 aa, Homo sapiens
ALWFSNHWV

SEQ ID NO: 143 - protein, 9 aa, Homo sapiens
ALWFSNHWV

SEQ ID NO: 144 - protein, 9 aa, Homo sapiens
ALWFSNHWV

SEQ ID NO: 145 - protein, 5 aa, Homo sapiens
TYGMS

SEQ ID NO: 146 - protein, 7 aa, Homo sapiens
GNTFTTY

SEQ ID NO: 147 - protein, 8 aa, Homo sapiens
GNTFTTYG

SEQ ID NO: 148 - protein, 17 aa, Homo sapiens
WINNYSGVSTYADDFKG

SEQ ID NO: 149 - protein, 6 aa, Homo sapiens
NNYSGV

SEQ ID NO: 150 - protein, 8 aa, Homo sapiens
INNYSGVS

SEQ ID NO: 151 - protein, 12 aa, Homo sapiens
DYYGSGGWVFDY

SEQ ID NO: 152 - protein, 12 aa, Homo sapiens
DYYGSGGWVFDY

SEQ ID NO: 153 - protein, 14 aa, Homo sapiens
ARDYYGSGGWVFDY

SEQ ID NO: 154 - protein, 16 aa, Homo sapiens
KSSQSLLDSEGKTYLN

SEQ ID NO: 155 - protein, 16 aa, Homo sapiens
KSSQSLLDSEGKTYLN

SEQ ID NO: 156 - protein, 11 aa, Homo sapiens
QSLLDSEGKTY

SEQ ID NO: 157 - protein, 7 aa, Homo sapiens
LVSKLDS

SEQ ID NO: 158 - protein, 7 aa, Homo sapiens
LVSKLDS

SEQ ID NO: 159 - protein, 3 aa, Homo sapiens
LVS

SEQ ID NO: 160 - protein, 9 aa, Homo sapiens
WQGTHFPRT

SEQ ID NO: 161 - protein, 9 aa, Homo sapiens
WQGTHFPRT

SEQ ID NO: 162 - protein, 9 aa, Homo sapiens
WQGTHFPRT

SEQ ID NO: 163 - protein, 5 aa, Mus musculus
SFAIS

SEQ ID NO: 164 - protein, 7 aa, Mus musculus
GISLSSF

SEQ ID NO: 165 - protein, 8 aa, Mus musculus
GISLSSFA

SEQ ID NO: 166 - protein, 16 aa, Mus musculus
VIWTGGGTDYNSALKS

SEQ ID NO: 167 - protein, 5 aa, Mus musculus
WTGGG

SEQ ID NO: 168 - protein, 7 aa, Mus musculus
IWTGGGT

SEQ ID NO: 169 - protein, 6 aa, Mus musculus
HWYLDV

SEQ ID NO: 170 - protein, 6 aa, Mus musculus
HWYLDV

SEQ ID NO: 171 - protein, 8 aa, Mus musculus
ASHWYLDV

SEQ ID NO: 172 - protein, 14 aa, Mus musculus
RSSTGAVSTSNYAN

SEQ ID NO: 173 - protein, 14 aa, Mus musculus
RSSTGAVSTSNYAN

SEQ ID NO: 174 - protein, 9 aa, Mus musculus
TGAVSTSNY

SEQ ID NO: 175 - protein, 7 aa, Mus musculus
GANSRAP

SEQ ID NO: 176 - protein, 7 aa, Mus musculus
GANSRAP

SEQ ID NO: 177 - protein, 3 aa, Mus musculus
GAN

SEQ ID NO: 178 - protein, 9 aa, Mus musculus
ALWFSNHWV

SEQ ID NO: 179 - protein, 9 aa, Mus musculus
ALWFSNHWV

SEQ ID NO: 180 - protein, 9 aa, Mus musculus
ALWFSNHWV

SEQ ID NO: 181 - protein, 5 aa, Mus musculus
TYGMS

SEQ ID NO: 182 - protein, 7 aa, Mus musculus
GNTFTTY

SEQ ID NO: 183 - protein, 8 aa, Mus musculus
GNTFTTYG

SEQ ID NO: 184 - protein, 17 aa, Mus musculus
WINNYSGVSTYADDFKG

SEQ ID NO: 185 - protein, 6 aa, Mus musculus
NNYSGV

SEQ ID NO: 186 - protein, 8 aa, Mus musculus
INNYSGVS

SEQ ID NO: 187 - protein, 12 aa, Mus musculus
DYYGSGGWVFDY

SEQ ID NO: 188 - protein, 12 aa, Mus musculus
DYYGSGGWVFDY

SEQ ID NO: 189 - protein, 14 aa, Mus musculus
ARDYYGSGGWVFDY

SEQ ID NO: 190 - protein, 16 aa, Mus musculus
KSSQSLLDSDGKTYLN

SEQ ID NO: 191 - protein, 16 aa, Mus musculus
KSSQSLLDSDGKTYLN

SEQ ID NO: 192 - protein, 11 aa, Mus musculus
QSLLDSDGKTY

SEQ ID NO: 193 - protein, 7 aa, Mus musculus
LVSKLDS

SEQ ID NO: 194 - protein, 7 aa, Mus musculus
LVSKLDS

SEQ ID NO: 195 - protein, 3 aa, Mus musculus
LVS

SEQ ID NO: 196 - protein, 9 aa, Mus musculus
WQGTHFPRT

SEQ ID NO: 197 - protein, 9 aa, Mus musculus
WQGTHFPRT

SEQ ID NO: 198 - protein, 9 aa, Mus musculus
WQGTHFPRT

SEQ ID NO: 199 - protein, 5 aa, Mus musculus
DYNMH

SEQ ID NO: 200 - protein, 7 aa, Mus musculus
GYTFIDY

SEQ ID NO: 201 - protein, 8 aa, Mus musculus
GYTFIDYN

SEQ ID NO: 202 - protein, 17 aa, Mus musculus
YIHPNNGGTSYNQKFKD

SEQ ID NO: 203 - protein, 6 aa, Mus musculus
HPNNGG

SEQ ID NO: 204 - protein, 8 aa, Mus musculus
IHPNNGGT

SEQ ID NO: 205 - protein, 13 aa, Mus musculus
SGIYYDYDSFFDY

SEQ ID NO: 206 - protein, 13 aa, Mus musculus
SGIYYDYDSFFDY

SEQ ID NO: 207 - protein, 15 aa, Mus musculus
SRSGIYYDYDSFFDY

SEQ ID NO: 208 - protein, 11 aa, Mus musculus
RASENIYSHLA

SEQ ID NO: 209 - protein, 11 aa, Mus musculus
RASENIYSHLA

SEQ ID NO: 210 - protein, 6 aa, Mus musculus
ENIYSH

SEQ ID NO: 211 - protein, 7 aa, Mus musculus
AATNLAD

SEQ ID NO: 212 - protein, 7 aa, Mus musculus
AATNLAD

SEQ ID NO: 213 - protein, 3 aa, Mus musculus
AAT

SEQ ID NO: 214 - protein, 9 aa, Mus musculus
QHFWGSPWT

SEQ ID NO: 215 - protein, 9 aa, Mus musculus
QHFWGSPWT

SEQ ID NO: 216 - protein, 9 aa, Mus musculus
QHFWGSPWT

SEQ ID NO: 217 - protein, 122 aa, Homo sapiens
QLQLQESGPGLVKPSETLSLTCAVSGYSISSGNYWGWIRQSPGKGLEWIGSVDHSGSTYYSPSLKSRVTISVDTSKNQFSLKLNSVTAADTADYYCARNTMIRGVMDWFDPWGQGTLVTVSS

SEQ ID NO: 218 - protein, 106 aa, Homo sapiens
AIQLTQSPSSLSASVGDRVTITCRASQGISSALAWYQQKPGKAPKVLIYDASSLESGVPSRFSGSGSGTDFTLTISSLQPEDFATYYCQQFRSYPTFGQGTKVEIK

SEQ ID NO: 219 - protein, 452 aa, Homo sapiens
QLQLQESGPGLVKPSETLSLTCAVSGYSISSGNYWGWIRQSPGKGLEWIGSVDHSGSTYYSPSLKSRVTISVDTSKNQFSLKLNSVTAADTADYYCARNTMIRGVMDWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 220 - protein, 213 aa, Homo sapiens
AIQLTQSPSSLSASVGDRVTITCRASQGISSALAWYQQKPGKAPKVLIYDASSLESGVPSRFSGSGSGTDFTLTISSLQPEDFATYYCQQFRSYPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 221 - protein, 122 aa, Homo sapiens
QLQLQESGPGLVKPSETLSLTCAVSGYSISSDYSWGWIRQPPGKGLEWIGSIYHSGNTYFNPSLKSRVTISVDTSKNQFSLKLSSVTAADTAVYYCARDKSYYFGPGSMDVWGQGTTVTVSS

SEQ ID NO: 222 - protein, 106 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFKSYLTFGQGTRLEIK

SEQ ID NO: 223 - protein, 452 aa, Homo sapiens
QLQLQESGPGLVKPSETLSLTCAVSGYSISSDYSWGWIRQPPGKGLEWIGSIYHSGNTYFNPSLKSRVTISVDTSKNQFSLKLSSVTAADTAVYYCARDKSYYFGPGSMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 224 - protein, 213 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFKSYLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 225 - protein, 123 aa, Homo sapiens
EVQLVESGGGLVQPGGSLRLSCAASGFTFSSYSMNWVRQAPGKGLEWVSYIISGSDTIFYADSVKGRFTISRDNAKNSLYLQMNSLRDEDTAVYYCARDETVVRGVINYFDYWGQGTLVTVSS

SEQ ID NO: 226 - protein, 106 aa, Homo sapiens
AIQLTQSPSSLSASVGDRVTITCRSSQGISSALAWYQQKPGKAPKLLIYDASSLESGVPSRFSGSGSGTDFTLTISSLQPEDFATYYCQQFISYPTFGQGTRLEIK

SEQ ID NO: 227 - protein, 453 aa, Homo sapiens
EVQLVESGGGLVQPGGSLRLSCAASGFTFSSYSMNWVRQAPGKGLEWVSYIISGSDTIFYADSVKGRFTISRDNAKNSLYLQMNSLRDEDTAVYYCARDETVVRGVINYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 228 - protein, 213 aa, Homo sapiens
AIQLTQSPSSLSASVGDRVTITCRSSQGISSALAWYQQKPGKAPKLLIYDASSLESGVPSRFSGSGSGTDFTLTISSLQPEDFATYYCQQFISYPTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 229 - protein, 117 aa, Homo sapiens
QVQLVQSGAEVKKPGASVKVSCKTSGNTQIRYYMYWVRQAPGQGLEWMGILNPNQDQTTYAQKFQGRVTMTRDTSTSTVYMELSSLRSEDTAVYYCATTYRYYMDVWGQGTTVTVSS

SEQ ID NO: 230 - protein, 108 aa, Homo sapiens
EIVLTQSPGTLSLSPGERATLSCRASQSVRSNYLAWYQQKPGQAPRLLIYGASSRATGIPDRFSGSGSGTDFTLTISRLEPEDFAVYYCQQYGSSPRTFGQGTKVEIK

SEQ ID NO: 231 - protein, 447 aa, Homo sapiens
QVQLVQSGAEVKKPGASVKVSCKTSGNTQIRYYMYWVRQAPGQGLEWMGILNPNQDQTTYAQKFQGRVTMTRDTSTSTVYMELSSLRSEDTAVYYCATTYRYYMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 232 - protein, 215 aa, Homo sapiens
EIVLTQSPGTLSLSPGERATLSCRASQSVRSNYLAWYQQKPGQAPRLLIYGASSRATGIPDRFSGSGSGTDFTLTISRLEPEDFAVYYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 233 - protein, 117 aa, Homo sapiens
QVQLVQSGAEVKKPGASVKVSCKTSGRTFIRYYMHWVRQAPGQGLEWMGIIWPNGDQTTYAQKFQGRVTMTRDTSTSTVYMELSSLRSEDTAVYYCATTYKYAMDVWGQGTTVTVSS

SEQ ID NO: 234 - protein, 108 aa, Homo sapiens
EIVLTQSPGTLSLSPGERATLSCRASQSVRSNYLAWYQQKPGQAPRLLIYGASSRATGIPDRFSGSGSGTDFTLTISRLEPEDFAVYYCQQYESPPRTFGQGTKVEIK

SEQ ID NO: 235 - protein, 447 aa, Homo sapiens
QVQLVQSGAEVKKPGASVKVSCKTSGRTFIRYYMHWVRQAPGQGLEWMGIIWPNGDQTTYAQKFQGRVTMTRDTSTSTVYMELSSLRSEDTAVYYCATTYKYAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 236 - protein, 215 aa, Homo sapiens
EIVLTQSPGTLSLSPGERATLSCRASQSVRSNYLAWYQQKPGQAPRLLIYGASSRATGIPDRFSGSGSGTDFTLTISRLEPEDFAVYYCQQYESPPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 237 - protein, 119 aa, Homo sapiens
QVQLQQWGAGLLKPSETLSLTCAVYGGSFSIYYWSWIRQPPGKGLELIGEINDEGNTNYNPSLKSRVTISVDTSKNQFSLKLSSVTAADTAVYYCARGGTGDIHAFDIWGQGTMVTVSS

SEQ ID NO: 238 - protein, 107 aa, Homo sapiens
DIQMTQSPSSLSASVGDRVTITCRASQGISKWLAWYQQKPEKAPKSLIYAASSLQSGVPSRFSGSGSGTDFTLTISSLQPEDFATYYCQQYNSYPWTFGQGTKVEIK

SEQ ID NO: 239 - protein, 449 aa, Homo sapiens
QVQLQQWGAGLLKPSETLSLTCAVYGGSFSIYYWSWIRQPPGKGLELIGEINDEGNTNYNPSLKSRVTISVDTSKNQFSLKLSSVTAADTAVYYCARGGTGDIHAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 240 - protein, 214 aa, Homo sapiens
DIQMTQSPSSLSASVGDRVTITCRASQGISKWLAWYQQKPEKAPKSLIYAASSLQSGVPSRFSGSGSGTDFTLTISSLQPEDFATYYCQQYNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 241 - protein, 118 aa, Homo sapiens
QVQLQQWGAGLLKPSETLSLTCAVYGGSFSGYYWSWLRQPPGKGLEWIGEISHSGSTNYNPSLKSRVTISVDTSKNQFSLKLSSVTAADTAVYYCARALSRYWYFDLWGRGTLVTVSS

SEQ ID NO: 242 - protein, 107 aa, Homo sapiens
EIVLTQSPATLSLSPGERATLSCRASQSASNYLAWYQQKPGQAPRLLIYDASNRATGIPARFSGSGSGTDFTLTISSLEPEDFAVYYCYQRSQWPISFGQGTRLEIK

SEQ ID NO: 243 - protein, 448 aa, Homo sapiens
QVQLQQWGAGLLKPSETLSLTCAVYGGSFSGYYWSWLRQPPGKGLEWIGEISHSGSTNYNPSLKSRVTISVDTSKNQFSLKLSSVTAADTAVYYCARALSRYWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 244 - protein, 214 aa, Homo sapiens
EIVLTQSPATLSLSPGERATLSCRASQSASNYLAWYQQKPGQAPRLLIYDASNRATGIPARFSGSGSGTDFTLTISSLEPEDFAVYYCYQRSQWPISFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 245 - protein, 114 aa, Homo sapiens
QVTLKESGPVLVKPTETLTLTCTVSGISLSSFAISWIRQPPGKALEWLAVIWTGGGTDYNSALKSRLTISKDTSKSQVVLTMTNMDPVDTATYYCASHWYLDVWGQGTTVTVSS

SEQ ID NO: 246 - protein, 109 aa, Homo sapiens
QTVVTQEPSFSVSPGGTVTLTCRSSTGAVSTSNYANWVQQTPGQAPRGLIGGANSRAPGIPDRFSGSILGNKAALTITGAQADDESDYYCALWFSNHWVFGGGTKLTVL

SEQ ID NO: 247 - protein, 444 aa, Homo sapiens
QVTLKESGPVLVKPTETLTLTCTVSGISLSSFAISWIRQPPGKALEWLAVIWTGGGTDYNSALKSRLTISKDTSKSQVVLTMTNMDPVDTATYYCASHWYLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 248 - protein, 215 aa, Homo sapiens
QTVVTQEPSFSVSPGGTVTLTCRSSTGAVSTSNYANWVQQTPGQAPRGLIGGANSRAPGIPDRFSGSILGNKAALTITGAQADDESDYYCALWFSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 249 - protein, 121 aa, Homo sapiens
QVQLVQSGSELKKPGASVKVSCKASGNTFTTYGMSWVRQAPGQGLEWMGWINNYSGVSTYADDFKGRFVFSLDTSVSTAYLQISSLKAEDTAVYYCARDYYGSGGWVFDYWGQGTTVTVSS

SEQ ID NO: 250 - protein, 112 aa, Homo sapiens
DVVMTQSPLSLPVTLGQPASISCKSSQSLLDSEGKTYLNWLQQRPGQSPRRLMYLVSKLDSGVPDRFSGSGSGTDFTLKISRVEAEDVGVYYCWQGTHFPRTFGGGTKVEIK

SEQ ID NO: 251 - protein, 451 aa, Homo sapiens
QVQLVQSGSELKKPGASVKVSCKASGNTFTTYGMSWVRQAPGQGLEWMGWINNYSGVSTYADDFKGRFVFSLDTSVSTAYLQISSLKAEDTAVYYCARDYYGSGGWVFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 252 - protein, 219 aa, Homo sapiens
DVVMTQSPLSLPVTLGQPASISCKSSQSLLDSEGKTYLNWLQQRPGQSPRRLMYLVSKLDSGVPDRFSGSGSGTDFTLKISRVEAEDVGVYYCWQGTHFPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 253 - protein, 114 aa, Mus musculus
QVQLKESGPGLVAPSQSLSITCTVSGISLSSFAISWVRQPPGKGLEWLGVIWTGGGTDYNSALKSRLTISKDTSKNQVFLKMNSLQTDDTARYYCASHWYLDVWGTGTTVTVSS

SEQ ID NO: 254 - protein, 109 aa, Mus musculus
QAVVTQESALTTSPGETVTLTCRSSTGAVSTSNYANWVQEKPDHLFTGLIGGANSRAPGIPARFSGSLIGDKAALTITGAQTEDEAIYFCALWFSNHWVFGGGTKLTVL

SEQ ID NO: 255 - protein, 121 aa, Mus musculus
QIQLVQSGPELKKPGETVKISCKASGNTFTTYGMSWVKQAPGKNLKWMGWINNYSGVSTYADDFKGRFAFSLETSATTAYLQINNLTNEDSATYFCARDYYGSGGWVFDYWGQGTTLTVSS

SEQ ID NO: 256 - protein, 112 aa, Mus musculus
DVVMTQTPLTLSVTIGQPASISCKSSQSLLDSDGKTYLNWLLQRPGQSPKRLMYLVSKLDSGVPDRFTGSGSGTDFTLKISRVEAEDLGVYYCWQGTHFPRTFGGGTKLEIK

SEQ ID NO: 257 - protein, 122 aa, Mus musculus
EVQLQQSRPDLVKPGASVKMSCKASGYTFIDYNMHWVKQRHGKSLEWIGYIHPNNGGTSYNQKFKDKATLTMNKSSSTAYMELRSLTSEDSAVYYCSRSGIYYDYDSFFDYWGQGTTLTVSS

SEQ ID NO: 258 - protein, 107 aa, Mus musculus
DIQMTQSPASLYVSVGETVTITCRASENIYSHLAWYQQKLGKSPHLLVYAATNLADGVPSRFSGSGSGTQYSLKINSLQSEDFGSYYCQHFWGSPWTFGGGTKLEIK

SEQ ID NO: 259 - protein, 999 aa, Homo sapien
MGPAPLPLLLGLFLPALWRRAITEAREEAKPYPLFPGPFPGSLQTDHTPLLSLPHASGYQPALMFSPTQPGRPHTGNVAIPQVTSVESKPLPPLAFKHTVGHIILSEHKGVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIRNSTAHSILISWVPGFDGYSPFRNCSIQVKEADPLSNGSVMIFNTSALPHLYQIKQLQALANYSIGVSCMNEIGWSAVSPWILASTTEGAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLIIFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 260 - protein, 1001 aa, Homo sapien
MGLAPLPLPLLLGLFLPALWSRAITEAREEAKPYPLFPGPLPGSLQTDHTSLLSLPHTSGYQPALMFSPTQPGRPYTGNVAIPRVTSAGSKLLPPLAFKHTVGHIILSEHKDVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIHNSTAHSILISWVPGFDGYSPFRNCSVQVKEVDPLSNGSVMIFNTSASPHMYQIKQLQALANYSIGVSCMNEIGWSAVSPWILASTTEGAPSVAPLNVTVFLNESRDNVDIRWMKPLTKRQAGELVGYRISHVWQSAGISKELLEEVGQNNSRAQISVQVHNATCTVRIAAVTKGGVGPFSDPVKIFIPAHGWVDHAPSSTPAPGNADPVLIIFGCFCGFILIGLVLYISLAVRKRVQETKFGNAFTEEDSELVVNYIAKKSFCRRAIELTLHSLGVSEELQNKLEDVVIDRNLLILGKILGEGEFGSVMEGNLKQEDGTSQKVAVKTMKLDNFSQREIEEFLSEAACMKDFSHPNVIRLLGVCIEMSSQGIPKPMVILPFMKYGDLHTYLLYSRLETGPKHIPLQTLLKFMMDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPNVRNQADVIYINTQLLESSEGLAEGSTLAPLDLNIDPDSIIASCSPHAAISVVTAEIHDSKPHEGRYILNGGSEEWEDVTSAASAAVTAEKNSVLPGERLVRNGVPWSHSSTLPLGSSLPDELLFADDSSESSEVLM

SEQ ID NO: 261 - protein, 994 aa, Mus musculus
MVLAPLLLGLLLLPALWSGGTAEKWEETELDQLFSGPLPGRLPVNHRPFSAPHSSRDQLPPPQTGRSHPAHTAAPQVTSTASKLLPPVAFNHTIGHIVLSEHKNVKFNCSINIPNTYQETAGISWWKDGKELLGAHHSITQFYPDEEGVSIIALFSIASVQRSDNGSYFCKMKVNNREIVSDPIYVEVQGLPYFIKQPESVNVTRNTAFNLTCQAVGPPEPVNIFWVQNSSRVNEKPERSPSVLTVPGLTETAVFSCEAHNDKGLTVSKGVHINIKVIPSPPTEVHILNSTAHSILVSWVPGFDGYSPLQNCSIQVKEADRLSNGSVMVFNTSASPHLYEIQQLQALANYSIAVSCRNEIGWSAVSPWILASTTEGAPSVAPLNITVFLNESNNILDIRWTKPPIKRQDGELVGYRISHVWESAGTYKELSEEVSQNGSWAQIPVQIHNATCTVRIAAITKGGIGPFSEPVNIIIPEHSKVDYAPSSTPAPGNTDSMFIILGCFCGFILIGLILCISLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLM